(19) United States
(12) Patent Application Publication
(10) Pub. No.: US 2012/0172281 A1
(43) Pub. Date: Jul. 5, 2012

(54) DETERGENT COMPOSITIONS COMPRISING MICROBIALLY PRODUCED FATTY ALCOHOLS AND DERIVATIVES THEREOF

(76) Inventors: Jeffrey John Scheibel, Loveland, OH (US); Phillip Richard Green, Wyoming, OH (US); Mathew Rude, S. San Francisco, CA (US)

(21) Appl. No.: 13/184,270

(22) Filed: Jul. 15, 2011

Related U.S. Application Data
(60) Provisional application No. 61/364,530, filed on Jul. 15, 2010.

(51) Int. Cl. C11D 1/14; C07C 33/025; C07C 305/14; C07C 43/04; C07C 43/15; C07C 305/08; C07C 31/125; C11D 3/20

(52) U.S. Cl. 510/493; 568/840; 568/909.5; 510/505; 568/671; 510/506; 568/687; 558/38

(57) ABSTRACT

Disclosed herein are detergent compositions comprising a microbially produced fatty alcohol or fatty alcohol derivative thereof. Further disclosed are cleaning compositions and personal care compositions comprising a microbially produced fatty alcohol or fatty alcohol derivative thereof. Methods of using the foregoing are also disclosed.

### CROSS REFERENCE TO RELATED APPLICATION

This application claims the benefit of U.S. Provisional Application No. 61/364,530, filed Jul. 15, 2010.

### BACKGROUND OF THE INVENTION

Fatty alcohols have many commercial uses. Fatty alcohols are used in the cosmetic and food industries, for example, as emulsifiers, emollients, and thickeners. Due to their amphiphilic nature, fatty alcohols behave as cosurfactants in some applications improving the foam characteristics of the formulations. Fatty alcohols are particularly useful in personal care and some household products, for example, detergents. In addition, fatty alcohols are used in waxes, gums, resins, pharmaceutical salves and lotions, lubricating oil additives, textile antistatic and finishing agents, plasticizers, cosmetics, industrial solvents, and solvents for fats.

One major use for fatty alcohols is for use in detergents per se and in the production of surfactants for use therein. Typically, conventional detergent compositions contain mixtures of various surfactants in order to remove a wide variety of soils and stains from surfaces. For example, various nonionic surfactants, especially the alkyl ethoxylates, are useful for removing greasy soils. In addition, fatty alcohols serve as starting materials in the preparation of other surfactants, such as fatty ether sulfates, fatty alcohol sulfates, fatty phosphate esters, alkylbenzyldimethylammonium salts, fatty amine oxides, alkyl polyglucosides, and alkyl glyceryl ether sulfonates.

Fatty alcohols may be derived from petroleum. Petroleum-based processes start with the Zeigler process, which uses petroleum-derived ethylene followed by hydroformylation of the resulting long chain mono-olefins, to produce fatty alcohols. Other processes use kerosene as a feedstock involving multiple steps include hydrogenation, sieve extraction of paraffins, partial dehydrogenation, hydroformylation and fractional distillation to recover the paraffins.

Obtaining specialty chemicals such as fatty alcohols from crude petroleum requires a significant capital investment as well as a great deal of energy. It is also an inefficient process because frequently the long chain hydrocarbons in crude petroleum are cracked to produce smaller monomers. These monomers are then used as the raw material to manufacture the more complex specialty chemicals.

Although it is possible to obtain fatty alcohols from natural oils and petroleum, it would be desirable to produce fatty alcohols from other sources, such as directly from renewable biomass.

### SUMMARY OF THE INVENTION

The invention provides a detergent composition comprising a microbially produced fatty alcohol or fatty alcohol derivative thereof.

Also provided is a cleaning composition comprising at least one microbially produced fatty alcohol or fatty alcohol derivative thereof, and a method for cleaning a targeted surface, the method including: providing a cleaning composition comprising a microbially produced fatty alcohol or a fatty alcohol derivative thereof, and contacting the targeted surface with the cleaning composition.

Further provided is a personal care composition comprising: at least one microbially produced fatty alcohol or fatty alcohol derivative thereof, and at least one silicone. A method of delivering personal care benefits to the hair or skin is also provided.

### BRIEF DESCRIPTION OF THE DRAWINGS

FIG. 1 is a schematic of a pathway for fatty alcohol production using carboxylic acid reductase.

FIG. 2 is a graphic representation of fatty alcohols produced by recombinant E. coli strains transformed with various plasmids.

FIG. 3A is a GC/MS trace of organic compounds produced by recombinant E. coli strains transformed with various plasmids.

FIG. 3B is another GC/MS trace of organic compounds produced by recombinant E. coli strains transformed with various plasmids.

FIG. 4 is a representation of a gel of PCR products from MG1655 wild-type cells, ΔfadD::cm cells, and ΔfadD cells.

FIG. 5A is a GC/MS trace of fatty alcohol production in MG1655(DE3, ΔfadD)/pETDuet-1-′tesA+pACYCDuet-1-carB cells. FIG. 5B is a GC/MS trace of fatty alcohol production in MG16655(DE3, ΔfadD, yjgB::kan)/pETDuet-1′ tesA+pACYCDuet-1-carB cells. FIG. 5C is a GC/MS trace of fatty alcohol production in MG16655(DE3, ΔfadD, yjgB::kan)/pDF1+pACYCDuet-1-carB cells.

FIG. 6 is a graphic representation of fatty alcohols produced by recombinant E. coli C41 (DE3, ΔfadE) strains transformed with plasmid pCDFDuet-1-fadD-acr1 or cotransformed with plasmids pCDFDuet-1-fadD-acr1 and pETDuet-1-′tesA. Total Ion Counts (TIC) of the different fatty alcohols are shown.

FIGS. 7A and 7B are GC-MS traces of fatty alcohol production using E. coli C41 (DE3, ΔfadE): (A) transformed with plasmid pCDFDuet-1-fadD-acr1, and (B) cotransformed with plasmids pCDFDuet-1-fadD-acr1 and pETDuet-1-′tesA, wherein the samples were reacted with trimethylsilane (TMS) imidazole. FIG. 7C is a mass spectrum of the peak at 9.017 min from FIG. 7B, which was identified as 1-trimethylsiloxytetradecane (=tetradecanol reacted with TMS imidazole), FIG. 7D is a mass spectrum of 1-trimethylsiloxytetradecane from the reference library.

FIG. 8 is a schematic of a pathway for fatty alcohol production using acyl-CoA reductase.

### DETAILED DESCRIPTION OF THE INVENTION

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein, including GenBank database sequences, are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DEFINITIONS

Throughout the specification, a reference may be made using an abbreviated gene name or polypeptide name, but it is understood that such an abbreviated gene or polypeptide name represents the genus of genes or polypeptides. Such gene names include all genes encoding the same polypeptide and homologous polypeptides having the same physiological function. Polypeptide names include all polypeptides and homologous polypeptides that have the same activity (e.g., that catalyze the same fundamental chemical reaction).

Unless otherwise indicated, the accession numbers referenced herein are derived from the NCBI database (National Center for Biotechnology Information) maintained by the National Institute of Health, U.S.A. Unless otherwise indicated, the accession numbers are as provided in the database as of October 2008.

EC numbers are established by the Nomenclature Committee of the International Union of Biochemistry and Molecular Biology (NC-IUBMB) (available at http://www.chein.qmuLac.ukhubmb/enzyme/). The EC numbers referenced herein are derived from the KEGG Ligand database, maintained by the Kyoto Encyclopedia of Genes and Genomics, sponsored in part by the University of Tokyo. Unless otherwise indicated, the EC numbers are as provided in the database as of October 2008.

The articles “a” and “an” are used herein to refer to one or to more than one (i.e., to at least one) object of the referenced article.

The term “alkyl” is used herein to mean a straight chain or branched hydrocarbon residue having from about 6 carbon atoms to about 26 carbon atoms and in the context of the present specification is used interchangeably with the term “fatty.”

The term “fatty alcohol” is used herein to refer to a compound comprising a hydrocarbon residue having about 6 carbon atoms or more and having a primary hydroxyl group. In some embodiments, the fatty alcohol is used herein to refer to a compound comprising a hydrocarbon residue having from about 6 carbon atoms to about 26 carbon atoms and having a primary hydroxyl group.

The term “fatty alcohol derivative” is used herein to mean a compound derived from a fatty alcohol as that term is defined herein. The fatty alcohol derivative can include the oxygen atom derived from the fatty alcohol, or, in some embodiments, does not include the aforesaid oxygen atom, in, for example, fatty amine oxides. For example, a fatty amide, which also can be referred to as an alkyl amide, refers to a compound comprising an amide group and a hydrocarbon residue having about 6 carbon atoms or more, wherein the hydrocarbon residue is bonded to the carbonyl group of the amide group or to the nitrogen atom of the amide group. In some embodiments, the fatty alcohol is used to refer to a compound comprising a hydrocarbon residue having from about 6 carbon atoms to about 26 carbon atoms, wherein the hydrocarbon residue is bonded to the carbonyl group of the amide group or to the nitrogen atom of the amide group. In some embodiments, the hydrocarbon residue is saturated. In other embodiments, the hydrocarbon residue is monounsaturated, and in yet other embodiments, the hydrocarbon residue is polyunsaturated.

As used herein, the term “alcohol dehydrogenase” (EC 1.1.1.*) refers to a polypeptide capable of catalyzing the conversion of a fatty aldehyde to an alcohol (e.g., fatty alcohol). Additionally, one of ordinary skill in the art will appreciate that some alcohol dehydrogenases will catalyze other reactions as well. For example, some alcohol dehydrogenases will accept other substrates in addition to fatty aldehydes. Such non-specific alcohol dehydrogenases are, therefore, also included in this definition. Nucleic acid sequences encoding alcohol dehydrogenases are known in the art, and such alcohol dehydrogenases are publicly available. Exemplary GenBank Accession Numbers are provided in Table A.

As used herein, the term “attenuate” means to weaken, reduce, or diminish. For example, a polypeptide can be attenuated by modifying the polypeptide to reduce its activity (e.g., by modifying a nucleotide sequence that encodes the polypeptide).

As used herein, the term “biomass” refers to any biological material from which a carbon source is derived. In some instances, a biomass is processed into a carbon source, which is suitable for bioconversion. In other instances, the biomass may not require further processing into a carbon source. The carbon source can be converted into a fatty alcohol. One exemplary source of biomass is plant matter or vegetation. For example, corn, sugar cane, or switchgrass can be used as biomass. Another non-limiting example of biomass is metabolic wastes, such as animal matter, for example cow manure. In addition, biomass may include algae and other marine plants. Biomass also includes waste products from industry, agriculture, forestry, and households. Examples of such waste products that can be used as biomass are fermentation waste, ensilage, straw, lumber, sewage, garbage, cellulosic urban waste, and food leftovers. Biomass also includes carbon sources such as carbohydrates (e.g., monosaccharides, disaccharides, or polysaccharides).

As used herein, the phrase “carbon source” refers to a substrate or compound suitable to be used as a source of carbon for prokaryotic or simple eukaryotic cell growth. Carbon sources can be in various forms, including, but not limited to polymers, carbohydrates, acids, alcohols, aldehydes, ketones, amino acids, peptides, and gases (e.g., CO and CO₂). These include, for example, various monosaccharides, such as glucose, fructose, mannose, and galactose; oligosaccharides, such as fructo-oligosaccharide and galacto-oligosaccharide; polysaccharides such as xylose and arabinose; disaccharides, such as sucrose, maltose, and turanose; cellulosic material, such as methyl cellulose and sodium carboxymethyl cellulose; saturated or unsaturated fatty acid esters, such as succinate, lactate, and acetate; alcohols, such as ethanol, methanol, and glycerol, or mixtures thereof. The carbon source can also be a product of photosynthesis, including, but not limited to, glucose. A preferred carbon source is biomass. Another preferred carbon source is glucose.

A nucleotide sequence is “complementary” to another nucleotide sequence if each of the bases of the two sequences matches (i.e., is capable of forming Watson-Crick base pairs). The term “complementary strand” is used herein interchangeably with the term “complement”. The complement of a nucleic acid strand can be the complement of a coding strand or the complement of a non-coding strand.

As used herein, the term “conditions sufficient to allow expression” means any conditions that allow a host cell to produce a desired product, such as a polypeptide or fatty alcohol described herein. Suitable conditions include, for example, fermentation conditions. Fermentation conditions can comprise many parameters, such as temperature ranges, levels of aeration, and media composition. Each of these conditions, individually and in combination, allow the host cell to grow. Exemplary culture media include broths or gels. Generally, the medium includes a carbon source, such as glucose, fructose, cellulose, or the like, that can be metabolized by a host cell directly. In addition, enzymes can be used in the medium to facilitate the mobilization (e.g., the depolymerization of starch or cellulose to fermentable sugars) and subsequent metabolism of the carbon source.

To determine if conditions are sufficient to allow expression, a host cell can be cultured, for example, for about 4, 8, 12, 24, 36, or 48 hours. During and/or after culturing, samples can be obtained and analyzed to determine if the conditions allow expression. For example, the host cells in the sample or the medium in which the host cells were grown can be tested for the presence of a desired product. When testing for the presence of a product, assays, such as, but not limited to, TLC, HPLC, GC/FID, GC/MS, LC/MS, and MS, can be used.

It is understood that the polypeptides described herein may have additional conservative or non-essential amino acid substitutions, which do not have a substantial effect on the polypeptide functions. Whether or not a particular substitution will be tolerated (i.e., will not adversely affect desired biological properties, such as carboxylic acid reductase activity) can be determined as described in Bowie et al., Science. 247: 1306-1310 (1990). A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenyl)alanine, methionine, tryptophan), beta-branched side chains (e.g., threonine, valine, isoleucine), and aromatic side chains (e.g., tyrosine, phenylalanine, tryptophan, histidine).

As used herein, “control element” means a transcriptional and/or a translational control element. Control elements include promoters and enhancers, such as ribosome binding sequences. The term “promoter element,” “promoter,” or “promoter sequence” refers to a DNA sequence that functions as a switch that activates the expression of a gene. If the gene is activated, it is said to be transcribed or participating in transcription. Transcription involves the synthesis of mRNA from the gene. A promoter, therefore, serves as a transcriptional regulatory element and also provides a site for initiation of transcription of the gene into mRNA. Control elements interact specifically with cellular proteins involved in transcription (Maniatis et al., Science, 236: 1237 (1987)).

As used herein, the term “fatty acid” means a carboxylic acid having the formula RCOOH. R represents an aliphatic group, preferably an alkyl group. R can comprise about 5 or more carbon atoms. In some embodiments, the fatty acid comprises between about 5 and about 24 carbon atoms. Fatty acids can be saturated, monounsaturated, or polyunsaturated. In addition, fatty acids can comprise a straight or branched chain. The branched chains may have one or more points of branching. In addition, the branched chains may include cyclic branches. In a preferred embodiment, the fatty acid is made from a fatty acid biosynthetic pathway.

As used herein, the term “fatty acid biosynthetic pathway” means a biosynthetic pathway that produces fatty acids. The fatty acid biosynthetic pathway includes fatty acid enzymes that can be engineered, as described herein, to produce fatty acids, and in some embodiments can be expressed with additional enzymes to produce fatty acids having desired carbon chain characteristics.

As used herein, the term “fatty acid derivative” means products made in part from the fatty acid biosynthetic pathway of the production host organism. “Fatty acid derivative” also includes products made in part from acyl-ACP or acyl-ACP derivatives. The fatty acid biosynthetic pathway includes fatty acid synthase enzymes which can be engineered as described herein to produce fatty acid derivatives, and in some examples can be expressed with additional enzymes to produce fatty acid derivatives having desired carbon chain characteristics. Exemplary fatty acid derivatives include, for example, fatty acids, acyl-CoA, fatty aldehyde, short and long chain alcohols, hydrocarbons, fatty alcohols, and esters (e.g., waxes, fatty acid esters, or fatty esters).

As used herein, the term “fatty acid derivative enzyme” means any enzyme that may be expressed or overexpressed in the production of fatty acid derivatives. These enzymes may be part of the fatty acid biosynthetic pathway. Non-limiting examples of fatty acid derivative enzymes include fatty acid synthases, thioesterases, acyl-CoA synthases, acyl-CoA reductases, alcohol dehydrogenases, alcohol acyltransferases, fatty alcohol-forming acyl-CoA reductases, carboxylic acid reductases (e.g., fatty acid reductases), acyl-ACP reductases, fatty acid hydroxylases, acyl-CoA desaturases, acyl-ACP desaturases, acyl-CoA oxidases, acyl-CoA dehydrogenases, ester synthases, and/or alkane biosynthetic polypeptides, etc. Fatty acid derivative enzymes can convert a substrate into a fatty acid derivative. In some examples, the substrate may be a fatty acid derivative that the fatty acid derivative enzyme converts into a different fatty acid derivative.

As used herein, “fatty acid enzyme” means any enzyme involved in fatty acid biosynthesis. Fatty acid enzymes can be expressed or overexpressed in host cells to produce fatty acids. Non-limiting examples of fatty acid enzymes include fatty acid synthases and thioesterases.

As used herein, “fatty aldehyde” means an aldehyde having the formula RCHO characterized by an unsaturated carbonyl group (C═O). In a preferred embodiment, the fatty aldehyde is any aldehyde made from a fatty acid or fatty acid derivative. In one embodiment, the R group is at least about 1, 2, 3, 4, 5, 6, 7, 8, 9, 10, 11, 12, 13, 14, 15, 16, 17, 18, 19, 20, 21, 22, 23, 24, 25, or 26 carbons in length, or is a value between any two of the foregoing values.

R can be straight or branched chain. The branched chains may have one or more points of branching. In addition, the branched chains may include cyclic branches.

Furthermore, R can be saturated or unsaturated. If unsaturated, the R can have one or more points of unsaturation.

In one embodiment, the fatty aldehyde is produced biosynthetically.

Fatty aldehydes have many uses. For example, fatty aldehydes can be used to produce many specialty chemicals. For example, fatty aldehydes are used to produce polymers, resins, dyes, flavorings, plasticizers, perfumes, pharmaceuticals, and other chemicals. Some are used as solvents, preservatives, or disinfectants. Some natural and synthetic compounds, such as vitamins and hormones, are aldehydes.

The terms “fatty aldehyde biosynthetic polypeptide”, “carboxylic acid reductase”, and “CAR” are used interchangeably herein.

As used herein, “fatty alcohol” means an alcohol having the formula ROH. In a preferred embodiment, the fatty alcohol is any alcohol made from a fatty acid or fatty acid derivative. In one embodiment, the R group is at least about 5, 6, 7, 8, 9, 10, 11, 12, 13, 14, 15, 16, 17, 18, 19, 20, 21, 22, 23, 24, 25, or 26 carbons in length, or is a value between any two of the foregoing values. Typically, the fatty alcohol comprises an R group that is 6 to 26 carbons in length. Preferably, the fatty alcohol comprises an R group that is 8, 10, 12, 14, 16, or 18 carbons in length.

R can be straight or branched chain. The branched chains may have one or more points of branching. In addition, the branched chains may include cyclic branches.

Furthermore, R can be saturated or unsaturated. If unsaturated, the R can have one or more points of unsaturation.

In one embodiment, the fatty alcohol is produced biosynthetically.

Fatty alcohols have many uses. For example, fatty alcohols can be used to produce many specialty chemicals. For example, fatty alcohols are used as a biofuel; as solvents for fats, waxes, gums, and resins; in pharmaceutical salves, emollients, and lotions; as lubricating-oil additives; in detergents and emulsifiers; as textile antistatic and finishing agents; as plasticizers; as nonionic surfactants; and in cosmetics, for example as thickeners.

“Gene knockout”, as used herein, refers to a procedure by which a gene encoding a target protein is modified or inactivated so to reduce or eliminate the function of the intact protein. Inactivation of the gene may be performed by general methods such as mutagenesis by UV irradiation or treatment with N-methyl-N′-nitro-N-nitrosoguanidine, site-directed mutagenesis, homologous recombination, insertion-deletion mutagenesis, or “Red-driven integration” (Datsenko et al., Proc. Natl. Acad. Sci. USA, 97: 6640-45 (2000)). For example, in one embodiment, a construct is introduced into a host cell, such that it is possible to select for homologous recombination events in the host cell. One of skill in the art can readily design a knock-out construct including both positive and negative selection genes for efficiently selecting transfected cells that undergo a homologous recombination event with the construct. The alteration in the host cell may be obtained, for example, by replacing through a single or double crossover recombination a wild type DNA sequence by a DNA sequence containing the alteration. For convenient selection of transformants, the alteration may, for example, be a DNA sequence encoding an antibiotic resistance marker or a gene complementing a possible auxotrophy of the host cell. Mutations include, but are not limited to, deletion-insertion mutations. An example of such an alteration includes a gene disruption (i.e., a perturbation of a gene) such that the product that is normally produced from this gene is not produced in a functional form. This could be due to a complete deletion, a deletion and insertion of a selective marker, an insertion of a selective marker, a frameshift mutation, an in-frame deletion, or a point mutation that leads to premature termination. In some instances, the entire mRNA for the gene is absent. In other situations, the amount of mRNA produced varies.

Calculations of “homology” between two sequences can be performed as follows. The sequences are aligned for optimal comparison purposes (e.g., gaps can be introduced in one or both of a first and a second amino acid or nucleotide sequence for optimal alignment and non-homologous sequences can be disregarded for comparison purposes). In a preferred embodiment, the length of a reference sequence that is aligned for comparison purposes is at least about 30%, preferably at least about 40%, more preferably at least about 50%, even more preferably at least about 60%, and even more preferably at least about 70%, at least about 80%, at least about 90%, or about 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the respective amino acid residue or nucleotide is identical at that position (as used herein, amino acid or nucleotide “identity” is equivalent to amino acid or nucleotide “homology”). The percent identity between the two sequences is a function of the number of identical positions shared by the sequences, taking into account the number of gaps and the length of each gap, which need to be introduced for optimal alignment of the two sequences.

The comparison of sequences and determination of percent homology between two sequences can be accomplished using a mathematical algorithm. In a preferred embodiment, the percent homology between two amino acid sequences is determined using the Needleman and Wunsch, J. Mol. Biol., 48: 444-453 (1970), algorithm that has been incorporated into the GAP program in the { }CG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent homology between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about which parameters should be applied to determine if a molecule is within a homology limitation of the claims) are a Blossum 62 scoring matrix with a gap penalty of 12, a gap extend penalty of 4, and a frameshift gap penalty of 5.

As used herein, a “host cell” is a cell used to produce a product described herein (e.g. a fatty alcohol described herein). A host cell can be modified to express or overexpress selected genes or to have attenuated expression of selected genes. Non-limiting examples of host cells include plant, animal, human, bacteria, yeast, or filamentous fungi cells.

As used herein, the term “hybridizes under low stringency, medium stringency, high stringency, or very high stringency conditions” describes conditions for hybridization and washing. Guidance for performing hybridization reactions can be found, for example, in Current Protocols in Molecular Biology, John Wiley & Sons, N.Y. (1989), 6.3.1-6.3.6. Aqueous and nonaqueous methods are described in that reference, and either method can be used. An example of hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2.×SSC, 0.1% SDS at 65° C.; and 4) very high stringency hybridization conditions in 0.5M sodium phosphate, 7% SDS at 65° C., followed by one or more washes at 0.2×SSC, 1% SDS at 65° C. Very high stringency conditions (4) are the preferred conditions unless otherwise specified.

The term “isolated” as used herein with respect to nucleic acids, such as DNA or RNA, refers to molecules separated from other DNAs or RNAs, respectively, that are present in the natural source of the nucleic acid. Moreover, by an “isolated nucleic acid” is meant to include nucleic acid fragments, which are not naturally occurring as fragments and would not be found in the natural state. The term “isolated” is also used herein to refer to polypeptides, which are isolated from other cellular proteins, and is meant to encompass both purified and recombinant polypeptides. The term “isolated” as used herein also refers to a nucleic acid or peptide that is substantially free of cellular material, viral material, or culture medium when produced by recombinant DNA techniques. The term “isolated” as used herein also refers to a nucleic acid or peptide that is substantially free of chemical precursors or other chemicals when chemically synthesized.

As used herein, the “level of expression of a gene in a cell” refers to the level of mRNA, pre-mRNA nascent transcript(s), transcript processing intermediates, mature mRNA(s), and degradation products encoded by the gene in the cell.

As used herein, the term “microorganism” means prokaryotic and eukaryotic microbial species from the domains Archaea, Bacteria, and Eucarya, the latter including yeast and filamentous fungi, protozoa, algae, or higher Protista. The terms “microbial cells” (i.e., cells from microbes) and “microbes” are used interchangeably and refer to cells or small organisms that can only be seen with the aid of a microscope.

As used herein, the term “nucleic acid” refers to polynucleotides, such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs.

As used herein, the term “operably linked” means that selected nucleotide sequence (e.g., encoding a polypeptide described herein) is in proximity to a promoter to allow the promoter to regulate expression of the selected DNA. In addition, the promoter is located upstream of the selected nucleotide sequence in terms of the direction of transcription and translation. By “operably linked” is meant that a nucleotide sequence and a regulatory sequence(s) are connected in such a way as to permit gene expression when the appropriate molecules (e.g., transcriptional activator proteins) are bound to the regulatory sequence(s).

The term “or” is used herein to mean, and is used interchangeably with, the term “and/or,” unless context clearly indicates otherwise.

As used herein, “overexpress” means to express or cause to be expressed a nucleic acid or polypeptide in a cell at a greater concentration than is normally expressed in a corresponding wild-type cell. For example, a polypeptide can be “overexpressed” in a recombinant host cell when the polypeptide is present in a greater concentration in the recombinant host cell compared to its concentration in a non-recombinant host cell of the same species.

As used herein, “partition coefficient” or “P” is defined as the equilibrium concentration of a compound in an organic phase divided by the concentration at equilibrium in an aqueous phase (e.g., fermentation broth). In one embodiment of a bi-phasic system described herein, the organic phase is formed by the fatty aldehyde or fatty alcohol during the production process. However, in some examples, an organic phase can be provided, such as by providing a layer of octane, to facilitate product separation. When describing a two phase system, the partition characteristics of a compound can be described as logP. For example, a compound with a logP of 1 would partition 10:1 to the organic phase: aqueous phase. A compound with a logP of −1 would partition 1:10 to the organic phase: aqueous phase. By choosing an appropriate fermentation broth and organic phase, a fatty aldehyde or fatty alcohol with a high logP value can separate into the organic phase even at very low concentrations in the fermentation vessel.

As used herein, the ter “purify,” “purified,” or “purification” means the removal or isolation of a molecule from its environment by, for example, isolation or separation. “Substantially purified” molecules are at least about 60% free, preferably at least about 75% free, and more preferably at least about 90% free from other components with which they are associated. As used herein, these terms also refer to the removal of contaminants from a sample. For example, the removal of contaminants can result in an increase in the percentage of fatty aldehyde or fatty alcohol in a sample. For example, when fatty alcohols are produced in a host cell, the fatty alcohols can be purified by the removal of host cell proteins. After purification, the percentage of fatty alcohols in the sample is increased.

The terms “purify,” “purified,” and “purification” do not require absolute purity. They are relative terms. Thus, for example, when fatty alcohols are produced in host cells, a purified fatty alcohol is one that is substantially separated from other cellular components (e.g., nucleic acids, polypeptides, lipids, carbohydrates, or other compounds). In another example, a purified fatty alcohol preparation is one in which the fatty alcohol is substantially free from contaminants, such as those that might be present following fermentation. In some embodiments, a fatty alcohol is purified when at least about 50% by weight of a sample is composed of the fatty alcohol. In other embodiments, a fatty alcohol is purified when at least about 60%, 70%, 80%, 85%, 90%, 92%, 95%, 98%, or 99% or more by weight of a sample is composed of the fatty alcohol.

As used herein, the term “recombinant polypeptide” refers to a polypeptide that is produced by recombinant DNA techniques, wherein generally DNA encoding the expressed protein or RNA is transferred into a suitable expression vector and that is in turn used to transform a host cell to produce the polypeptide or RNA.

As used herein, the term “substantially identical” (or “substantially homologous”) is used to refer to a first amino acid or nucleotide sequence that contains a sufficient number of identical or equivalent (e.g., with a similar side chain, such as involving conservative amino acid substitutions) amino acid residues or nucleotides to a second amino acid or nucleotide sequence such that the first and second amino acid or nucleotide sequences have similar activities.

As used herein, the term “synthase” means an enzyme which catalyzes a synthesis process.

As used herein, the term synthase includes synthases, synthetases, and ligases.

As used herein, the term “transfection” means the introduction of a nucleic acid (e.g., via an expression vector) into a recipient cell by nucleic acid-mediated gene transfer.

As used herein, “transformation” refers to a process in which a cell's genotype is changed as a result of the cellular uptake of exogenous DNA or RNA. This may result in the transformed cell expressing a recombinant form of an RNA or polypeptide. In the case of antisense expression from the transferred gene, the expression of a naturally-occurring form of the polypeptide is disrupted.

As used herein, a “transport protein” is a polypeptide that facilitates the movement of one or more compounds in and/or out of a cellular organelle and/or a cell.

As used herein, a “variant” of polypeptide X refers to a polypeptide having the amino acid sequence of peptide X in which one or more amino acid residues is altered. The variant may have conservative changes or nonconservative changes. Guidance in determining which amino acid residues may be substituted, inserted, or deleted without affecting biological activity may be found using computer programs well known in the art, for example, LASERGENE software (DNASTAR).

The term “variant,” when used in the context of a polynucleotide sequence, may encompass a polynucleotide sequence related to that of a gene or the coding sequence thereof. This definition may also include, for example, “allelic,” “splice,” “species,” or “polymorphic” variants. A splice variant may have significant identity to a reference polynucleotide, but will generally have a greater or fewer number of polynucleotides due to alternative splicing of exons during mRNA processing. The corresponding polypeptide may possess additional functional domains or an absence of domains. Species variants are polynucleotide sequences that vary from one species to another. The resulting polypeptides generally will have significant amino acid identity relative to each other. A polymorphic variant is a variation in the polynucleotide sequence of a particular gene between individuals of a given species.

As used herein, the term “vector” refers to a nucleic acid molecule capable of transporting another nucleic acid to which it has been linked. One type of useful vector is an episome (i.e., a nucleic acid capable of extra-chromosomal replication). Useful vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids,” which refer generally to circular double stranded DNA loops that, in their vector form, are not bound to the chromosome. In the present specification, “plasmid” and “vector” are used interchangeably, as the plasmid is the most commonly used form of vector. However, also included are such other forms of expression vectors that serve equivalent functions and that become known in the art subsequently hereto.

### Microbial Production of Fatty Alcohols or Fatty Alcohol Derivatives Thereof

The invention provides a detergent composition comprising a microbially produced fatty alcohol or derivative thereof (i.e., fatty alcohol derivative thereof). The detergent compositions may contain solely those fatty alcohols or derivatives thereof that are microbially produced (i.e., all of the fatty alcohols or derivatives thereof are microbially produced), or may optionally and additionally contain a fatty alcohol or derivative thereof that is produced by any other means, including for example synthetic means (i.e., some of the fatty alcohols or derivatives thereof are microbially produced).

The microbially produced fatty alcohol or fatty alcohol derivative thereof is prepared by a method comprising expressing in a host cell a gene encoding a polypeptide comprising an amino acid sequence set forth in “Sequence Listings 2” or a variant thereof, to produce the fatty alcohol, and isolating the fatty alcohol from the host cell.

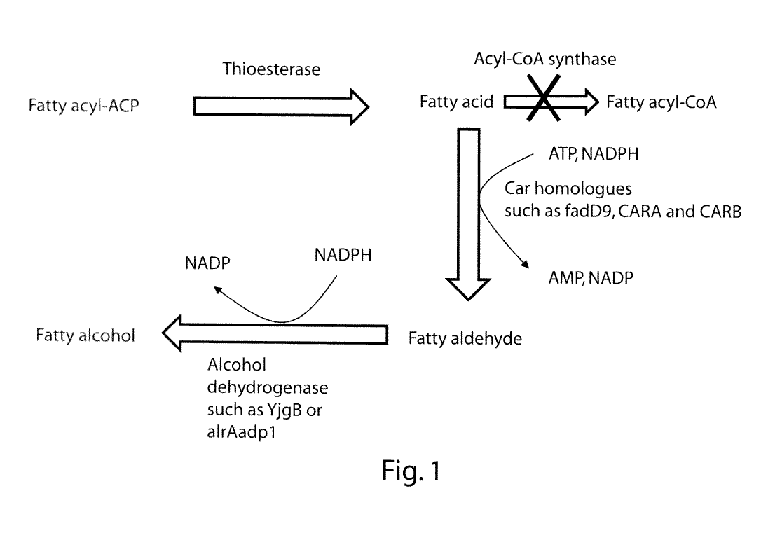

The fatty alcohols can be produced by a biosynthetic pathway depicted in FIG. 1. In this pathway, a fatty acid is first activated by ATP and then reduced by a carboxylic acid reductase (CAR)-like enzyme (e.g., CarA, CarB, or FadD9) to generate a fatty aldehyde. The fatty aldehyde can then be further reduced into a fatty alcohol by an alcohol dehydrogenase(s), such as AlrAadp1 or YjgB. As demonstrated herein, YjgB may be the presumed alcohol dehydrogenase, whose substrates include fatty aldehydes, for example, fatty aldehydes with carbon chain lengths from C₈ to C₁₈.

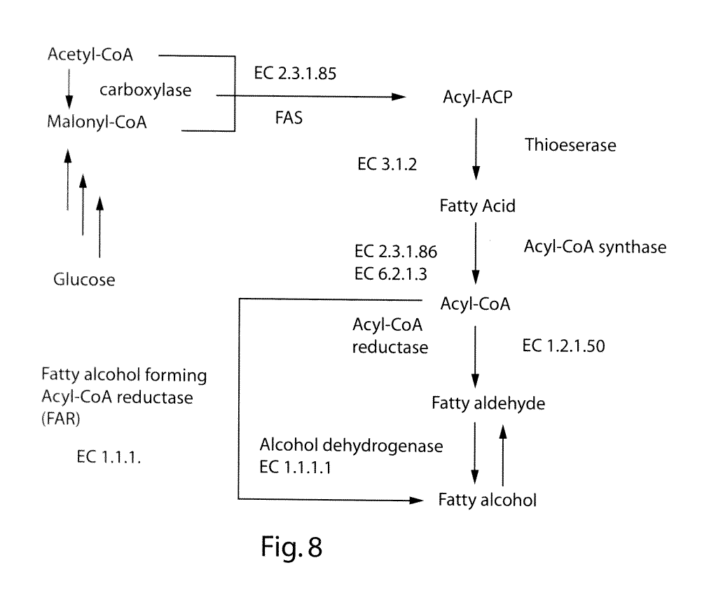

The fatty alcohols also can be produced by a biosynthetic pathway depicted in FIG. 8. In this pathway, a fatty acid is first activated by an acyl-CoA synthase (e.g., FadD) and then reduced by an acyl-CoA reductase (e.g., Acr1) to generate a fatty aldehyde. The fatty aldehyde can then be further reduced into a fatty alcohol by an alcohol dehydrogenase(s) (e.g., AlrAadp1 or YjgB).

### Substrates for Fatty Alcohol Production

The compositions and methods described herein can be used to produce fatty alcohols, for example, from fatty aldehydes, which themselves can be produced from an appropriate substrate. While not wishing to be bound by theory, it is believed that the fatty aldehyde biosynthetic polypeptides described herein produce fatty aldehydes from substrates via a reduction mechanism. In some instances, the substrate is a fatty acid derivative (e.g., a fatty acid), and a fatty aldehyde having particular branching patterns and carbon chain length can be produced from a fatty acid derivative having those characteristics that would result in a particular fatty aldehyde. Through an additional reaction mechanism, the fatty aldehyde can be converted into the desired fatty alcohol.

Accordingly, each step within a biosynthetic pathway that leads to the production of a fatty acid derivative substrate can be modified to produce or overproduce the substrate of interest. For example, known genes involved in the fatty acid biosynthetic pathway or the fatty aldehyde pathway can be expressed, overexpressed, or attenuated in host cells to produce a desired substrate (see, e.g., PCT Publication No, WO 2008/119082). Exemplary genes are provided in Table A.

TABLE A
Accession Numbers are from NCBI. GenBank. Release 159.0 as of March 2008
EC Numbers are from KEGG, Release 42.0 as of April 2007 (plus daily updates up to March 2008)

| | | | | | | | |
|---|---|---|---|---|---|---|---|
| | | | | EC | | | |
| CATEGORY | GENE | NAME | ACCESSION | NUMBER | MODIFICATION | USE | ORGANISM |

1. Fatty Acid Production Increase/Product Production Increase
increase acyl-CoA
reduce catabolism of derivatives and intermediates
reduce feedback inhibition
attenuate other pathways that consume fatty acids

| | | | | | | |
|---|---|---|---|---|---|---|
| accA | Acetyl-CoA carboxylase, | AAC73296, | 6.4.1.2 | Over-express | increase | Escherichia coli, Lactococci |
| | subunit A | NP_414727 | | | Malonyl-CoA | |
| | (carboxyltransferase | | | | production | |
| | alpha) | | | | | |
| accB | Acetyl-CoA carboxylase, | NP_417721 | 6.4.1.2 | Over-express | increase | Escherichia coli, Lactococci |
| | subunit B (BCCP: biotin | | | | Malonyl-CoA | |
| | carboxyl carrier protein) | | | | production | |
| accC | Acetyl-CoA carboxylase, | NP_417722 | 6.4.1.2, | Over-express | increase | Escherichia coli, Lactococci |
| | subunit C (biotin | | 6.3.4.14 | | Malonyl-CoA | |
| | carboxylase) | | | | production | |
| accD | Acetyl-CoA carboxylase, | NP_416819 | 6.4.1.2 | Over express | increase | Escherichia coli, Lactococci |
| | subunit D | | | | Malonyl-CoA | |
| | (carboxyltransferase beta) | | | | production | |
| aceE | pyruvate dehydrogenase, | NP_414656, | 1.2.4.1 | Over express | increase | Escherichia coli |
| | subunit E1 | AAC73226 | | | Acetyl-CoA | |
| | | | | | production | |
| aceF | pyruvate dehydrogenase, | NP_414657 | 2.3.1.12 | Over-express | increase | Escherichia coli |
| | subunit E2 | | | | Acetyl-CoA | |
| | | | | | production | |
| ackA | acetate kinase | AAC75356, | 2.7.2.1 | Delete or reduce | increase | Escherichia coli |
| | | NP_416799 | | | Acetyl-CoA | |
| | | | | | production | |
| ackB | acetate kinase AckB | BAB81430 | 2.7.2.1 | Delete or reduce | increase | Escherichia coli |
| | | | | | Acetyl-CoA | |
| | | | | | production | |
| acpP | acyl carrier protein | AAC74178 | NONE | Over-express | increase | Escherichia coli |
| | | | | | Acetyl-CoA | |
| | | | | | production | |
| fadD | acyl-CoA synthase | AP_002424 | 2.3.1.86, | Over-express | increase Fatty | Escherichia coli W3110 |
| | | | 6.2.1.3 | | acid production | |
| adhE | alcohol dehydrogenase | CAA47743 | 1.1.1.1, | Delete or reduce | increase | Escherichia coli W3111 |
| | | | 1.2.1.10 | | Acetyl-CoA | |
| | | | | | production | |
| cer1 | Aldehyde decarbonylase | BAA11024 | 4.1.99.5 | Over-express | increase | Arabidopsis thaliana |
| | | | | | Acetyl-CoA | |
| | | | | | production | |
| fabA | beta-hydroxydecanoyl | NP_415474 | 4.2.1.60 | express | fatty acyl-CoA | E. coli K12 |
| | thioester dehydrase | | | | production | |
| fabD | [acyl-carrier-protein] | AAC74176 | 2.3.1.39 | Over-express | increase | E. coli K12 |
| | S-malonyltransferase | | | | Acetyl-CoA | |
| | | | | | production | |
| fabF | 3-oxoacyl-[acyl-carrier-protein] | AAC74179 | 2.3.1.179 | Delete or | increase | E. coli K12 |
| | synthase II | | | OverExpress | Acetyl-CoA | |
| | | | | | production | |
| fabG | 3-oxoacyl-[acyl-carrier | AAC74177 | 1.1.1.100 | Over-express | increase | E. coli K12 |
| | protein] reductase | | | | Acetyl-CoA | |
| | | | | | production | |
| fabH | 3-oxoacyl-[acyl-carrier-protein] | AAC74175 | 2.3.1.180 | Over-express | increase | E. coli K12, lactococci |
| | synthase III | | | | Acetyl-CoA | |
| | | | | | production | |
| fabI | enoyl-[acyl-carrier-protein] | NP_415804 | 1.3.1.9 | express | fatty acyl-CoA | E. coli K12, lactococci |
| | reductase, | | | | production | |
| | NADH-dependent | | | | | |
| fabR | Transcriptional Repressor | NP_418398 | NONE | Delete or reduce | modulate | E. coli K12 |
| | | | | | unsaturated fatty | |
| | | | | | acid production | |
| fabZ | (3R)-hydroxymyristol | NP_414722 | 4.2.1.— | | | E. coli K12 |
| | acyl carrier protein | | | | | |
| | dehydratase | | | | | |
| fade | acyl-CoA dehydrogenase | AAC73325 | 1.3.99.3, | Delete or reduce | increase | |
| | | | 1.3.99.— | | Acetyl-CoA | |
| | | | | | production | |
| acr1 | Fatty Acyl-CoA reductase | YP_047869, | 1.2.1.42 | Over-express | for fatty alcohol | Acinetobacter sp., i.e. calcoaceticus |
| | | AAC45217 | | | production | |
| GST, gshB | Glutathione synthase | P04425 | 6.3.2.3 | Delete or reduce | increase | E. coli K12 |
| | | | | | Acyl-CoA | |
| gpsA | biosynthetic sn-glycerol | AAC76632, | EC: | Delete or reduce | increase | E. coli K12 |
| | 3-phosphate | NP_418065 | 1.1.1.94 | | Acetyl-CoA | |
| | dehydrogenase | | | | production | |
| ldhA | lactate dehydrogenase | AAC74462, | EC: | Delete or reduce | increase | E. coli K12 |
| | | NP_415898 | 1.1.1.27, | | Acetyl-CoA | |
| | | | 1.1.1.28 | | production | |
| Lipase | Triglyceride Lipase | CAA89087, | 3.1.1.3 | express | increase Fatty | Saccharomyces cerevisiae |
| | | CAA98876 | | | acid production | |
| | Malonyl-CoA | AAA26500 | 4.1.1.9, | Over-express | | Saccharopolyspora erythraea |
| | decarboxylase | | 4.1.1.41 | | | |
| panD | aspartate 1-decarboxylase | BAB96708 | 4.1.1.11 | Over-express | increase | Escherichia coli W3110 |
| | | | | | Acyl-CoA | |
| panK a.k.a. coaA | pantothenate kinase | AAC76952 | 2.7.1.33 | Over-express | increase | E. coli |
| | | | | | Acetyl-CoA | |
| | | | | | production | |
| panK a.k.a. coaA, | pantothenate kinase | AAC76952 | 2.7.1.33 | Express, | increase | E. coli |
| R106K | | | | Over-express, | Acetyl-CoA | |
| | | | | R106K mutation | production | |
| Pdh | Pyruvate dehydrogenase | BAB34380, | 1.2.4.1 | Over-express | increase | |
| | | AAC73226, | | | Acetyl-CoA | |
| | | NP_415392 | | | production | |
| pflB | formate acetyltransferase | AAC73989, | EC: | Delete or reduce | increase | |
| | (pyruvate formate lyase) | P09373 | 2.3.1.54 | | Acetyl-CoA | |
| | | | | | production | |
| plsB | acyltransferase | AAC77011 | 2.3.1.15 | D311E mutation | reduce limits on | E. coli K12 |
| | | | | | Acyl-CoA pool | |
| poxB | pyruvate oxidase | AAC73958, | 1.2.2.2 | Delete or reduce | increase | |
| | | NP_415392 | | | Acetyl-CoA | |
| | | | | | production | |
| Pta | phosphotransacetylase | AAC75357, | 2.3.1.8 | Delete or reduce | increase | |
| | | NP_416800 | | | Acetyl-CoA | |
| | | | | | production | |
| udhA | pyridine nucleotide | CAA46822 | 1.6.1.1 | Over-express | conversion | |
| | transhydrogenase | | | | NADH to | |
| | | | | | NADPH or vice | |
| | | | | | versa | |
| fadB | fused 3-hydroxybutyryl-CoA | AP_003956 | 4.2.1.17, | Delete or reduce | Block fatty acid | E. coli |
| | epimerase/delta(3)-cis-delta | | 5.1.2.3, | | degradation | |
| | (2)-trans-enoyl-CoA | | 5.3.3.8, | | | |
| | isomerase/enoyl-CoA | | 1.1.1.35 | | | |
| | hydratase and | | | | | |
| | 3-hydroxyacyl-CoA | | | | | |
| | dehydrogenase | | | | | |
| fadJ | 3-hydroxyacyl-CoA | AAC75401 | 1.1.1.35, | Delete or reduce | Block fatty acid | E. coli |
| | dehydrogenase; K01692 | | 4.2.1.17, | | degradation | |
| | enoyl-CoA hydratase; | | 5.1.2.3 | | | |
| | K01782 | | | | | |
| | 3-hydroxybutyryl-CoA | | | | | |
| | epimerase | | | | | |
| fadA | 3-ketoacyl-CoA thiolase | BAE77458 | 2.3.1.16 | Delete or reduce | Block fatty acid | E. coli |
| | | | | | degradation | |
| fadI | beta-ketoacyl-CoA | AAC75402 | 2.3.1.16 | Delete or reduce | Block fatty acid | E. coli |
| | thiolase | | | | degradation | |
| YdiO | acyl-coA dehydrogenase | YP_852786 | 1.3.99.— | Delete or reduce | Block fatty acid | E. coli |
| | | | | | degradation | |

2. Structure Control
2A. Chain Length Control

| | | | | | | | |
|---|---|---|---|---|---|---|---|
| 2 | teas | thioesterase | P0ADA1 | 3.1.2.—, | Delete and/or | C18 Chain Length | |
| | | | | 3.1.1.5 | express | | |
| | tesA without | thioesterase | AAC73596, | 3.1.2.—, | express or | C18:1 | E. coli |
| | leader sequence | | NP_415027 | 3.1.1.5 | overexpress | | |
| | tesA without | thioesterase | P0ADA1 | 3.1.2.—, | Express and/or | <C18 Chain | E. coli |
| | leader | | | 3.1.1.5 | overexpress | Length | |
| | sequence:L109P | | | | mutation L109P | | |
| | fatB1 | thioesterase | Q41635 | 3.1.2.14 | express or | C12:0 | Umbellularia californica |
| | (umbellularia) | | | | overexpress | | |
| | fatB2 | thioesterase | AAC49269 | 3.1.2.14 | express or | C8:0-C10:0 | Cuphea hookeriana |
| | (umbellularia)DELETE | | | | overexpress | | |
| | umbelluria) | | | | | | |
| | fatB3 | thioesterase | AAC72881 | 3.1.2.14 | express or | C14:0-C16:0 | Cuphea hookeriana |
| | | | | | overexpress | | |
| | fatB | thioesterase | Q39473 | 3.1.2.14 | express of | C14:0 | Cinnamomum camphora |
| | (cinnamonum) | | | | overexpress | | |
| | fatB[M141T]* | thioesterase | CAA85388 | 3.1.2.14 | express or | C16:1 | Arabidopsis thaliana |
| | | | | | overexpress | | |
| | fatA1 (Helianthus) | thioesterase | AAL79361 | 3.1.2.14 | express or | C18:1 | Helianthus annuus |
| | | | | | overexpress | | |
| | Atfata | thioesterase | NP_189147, | 3.1.2.14 | express or | C18:1 | Arabidopsis thaliana |
| | (ARABIDOPSIS | | NP_193041 | | overexpress | | |
| | FATA | | | | | | |
| | ACYL-ACP | | | | | | |
| | THIOESTERASE) | | | | | | |
| | fatA | thioesterase | CAC39106 | 3.1.2.14 | express or | C18:1 | Brassica juncea |
| | | | | | overexpress | | |
| | fatA (cuphea) | thioesterase | AAC72883 | 3.1.2.14 | express or | C18:1 | Cuphea hookeriana |
| | | | | | overexpress | | |

2B. Branching Control

| | | | | | | |
|---|---|---|---|---|---|---|
| attenuate FabH | | | | | | |
| express FabH | | | | | increase branched | |
| from S. glaucescens | | | | | chain fatty acid | |
| or S. coelicolor | | | | | derivatives | |
| and | | | | | | |
| knock out | | | | | | |
| endogenouse | | | | | | |
| FabH | | | | | | |
| express FabH | | | | | | |
| from B. subtilis | | | | | | |
| and knock out | | | | | | |
| endogenouse | | | | | | |
| FabH | | | | | | |
| bdk-E3- | | | EC 1.2.4.4 | | | |
| dihydroplipoyl | | | | | | |
| dehyrodgenase | | | | | | |
| subunit | | | | | | |
| bkd-E1- | decarboxylase subunits of | | EC 1.2.4.4 | | | |
| alpha/beta subunit | branched-chain α-keto | | | | | |
| | acid dehydrogenase | | | | | |
| | complex | | | | | |
| bkd-E2- | | | EC 1.2.4.4 | | | |
| dihydrolipoyl | | | | | | |
| transacylase | | | | | | |
| subunit | | | | | | |
| bkdA1 | branched-chain α-keto | NP_628006 | EC 1.2.4.4 | express or | make | Streptomyces coelicolor |
| | acid dehydrogenase | | | Over-Express | branched-chain | |
| | a-subunit (E1a) | | | | acyl-CoA | |
| | | | | | precursors | |
| bkdB1 | branched-chain α-keto | NP_628005 | EC 1.2.4.4 | express or | make | Streptomyces coelicolor |
| | acid dehydrogenase | | | Over-Express | branched-chain | |
| | a-subunit (E1b) | | | | acyl-CoA | |
| | | | | | precursors | |
| bkdC1 | dihydrolipoyl | NP_628004 | EC | express or | make | Streptomyces coelicolor |
| | transacetylase (E2) | | 2.3.1.168 | Over-Express | branched-chain | |
| | | | | | acyl-CoA | |
| | | | | | precursors | |
| bkdA2 | branched-chain a-ketoacid | NP_733618 | EC 1.2.4.4 | express or | make | Streptomyces coelicolor |
| | dehydrogenase a-subunit | | | Over-Express | branched-chain | |
| | (E1a) | | | | acyl-CoA | |
| | | | | | precursors | |
| bkdB2 | branched-chain a-ketoacid | NP_628019 | EC 1.2.4.4 | express or | make | Streptomyces coelicolor |
| | dehydrogenase b-subunit | | | Over-Express | branched-chain | |
| | (E1b) | | | | acyl-CoA | |
| | | | | | precursors | |
| bkdC2 | dihydrolipoyl | NP_628018 | EC | express or | make | Streptomyces coelicolor |
| | transacetylase (E2) | | 2.3.1.168 | Over-Express | branched-chain | |
| | | | | | acyl-CoA | |
| | | | | | precursors | |
| bkdA | branched-chain a-ketoacid | BAC72074 | EC 1.2.4.4 | express or | make | Streptomyces avermitilis |
| | dehydrogenase a-subunit | | | Over-Express | branched-chain | |
| | (E1a) | | | | acyl-CoA | |
| | | | | | precursors | |
| bkdB | branched-chain a-ketoacid | BAC72075 | EC 1.2.4.4 | express or | make | Streptomyces avermitilis |
| | dehydrogenase b-subunit | | | Over-Express | branched-chain | |
| | (E1b) | | | | acyl-CoA | |
| | | | | | precursors | |
| bkdC | dihydrolipoyl | BAC72076 | EC | express or | make | Streptomyces avermitilis |
| | transacetylase (E2) | | 2.3.1.168 | Over-Express | branched-chain | |
| | | | | | acyl-CoA | |
| | | | | | precursors | |
| bkdF | branched-chain a-ketoacid | BAC72088 | EC 1.2.4.4 | express or | make | Streptomyces avermitilis |
| | dehydrogenase a-subunit | | | Over-Express | branched-chain | |
| | (E1a) | | | | acyl-CoA | |
| | | | | | precursors | |
| bkdG | branched-chain a-ketoacid | BAC72089 | EC 1.2.4.4 | express or | make | Streptomyces avermitilis |
| | dehydrogenase b-subunit | | | Over-Express | branched-chain | |
| | (E1b) | | | | acyl-CoA | |
| | | | | | precursors | |
| bkdH | dihydrolipoyl | BAC72090 | EC | express or | make | Streptomyces avermitilis |
| | transacetylase (E2) | | 2.3.1.168 | Over-Express | branched-chain | |
| | | | | | acyl-CoA | |
| | | | | | precursors | |
| bkdAA | branched-chain a-ketoacid | NP_390285 | EC 1.2.4.4 | express or | make | Bacillus subtilis |
| | dehydrogenase a-subunit | | | Over-Express | branched-chain | |
| | (E1a) | | | | acyl-CoA | |
| | | | | | precursors | |
| bkdAB | branched-chain a-ketoacid | NP_390284 | EC 1.2.4.4 | express or | make | Bacillus subtilis |
| | dehydrogenase b-subunit | | | Over-Express | branched-chain | |
| | (E1b) | | | | acyl-CoA | |
| | | | | | precursors | |
| bkdB | dihydrolipoyl | NP_390283 | EC | express or | make | Bacillus subtilis |
| | transacetylase (E2) | | 2.3.1.168 | Over-Express | branched-chain | |
| | | | | | acyl-CoA | |
| | | | | | precursors | |
| bkdA1 | branched-chain a-ketoacid | AAA65614 | EC 1.2.4.4 | express or | make | Pseudomonas putida |
| | dehydrogenase a-subunit | | | Over-Express | branched-chain | |
| | (E1a) | | | | acyl-CoA | |
| | | | | | precursors | |
| bkdA2 | branched-chain a-ketoacid | AAA65615 | EC 1.2.4.4 | express or | make | Pseudomonas putida |
| | dehydrogenase b-subunit | | | Over-Express | branched-chain | |
| | (E1b) | | | | acyl-CoA | |
| | | | | | precursors | |
| bkdC | dihydrolipoyl | AAA65617 | EC | express or | make | Pseudomonas putida |
| | transacetylase (E2) | | 2.3.1.168 | Over-Express | branched-chain | |
| | | | | | acyl-CoA | |
| | | | | | precursors | |
| Lpd | dihydrolipoamide | NP_414658 | 1.8.1.4 | express or | make | Escherichia coli |
| | dehydrogenase (E3) | | | Over-Express | branched-chain | |
| | | | | | acyl-CoA | |
| | | | | | precursors | |
| IlvE | branched-chain amino | YP_026247 | 2.6.1.42 | express or | make branched | Escherichia coli |
| | acid aminotransferase | | | Over-Express | a-ketoacids | |
| IlvE | branched-chain amino | AAF34406 | 2.6.1.42 | express or | make branched | Lactococcus lactis |
| | acid aminotransferase | | | Over-Express | a-ketoacids | |
| IlvE | branched-chain amino | NP_745648 | 2.6.1.42 | express or | make branched | Pseudomonas putida |
| | acid aminotransferase | | | Over-Express | a-ketoacids | |
| IlvE | branched-chain amino | NP_629657 | 2.6.1.42 | express or | make branched | Streptomyces coelicolor |
| | acid aminotransferase | | | Over-Express | a-ketoacids | |
| Ccr | crotonyl-CoA reductase | NP_630556 | 1.6.5.5.1.1.1.1 | express or | Converting | Streptomyces coelicolor |
| | | | | Over Express | crotonyl-CoA to | |
| | | | | | butyryl-CoA | |
| Ccr | crotonyl-CoA reductase | AAD53915 | 1.6.5.5.1.1.1.1 | express or | Converting | Streptomyces cinnamonensis |
| | | | | Over Express | crotonyl-CoA to | |
| | | | | | butyryl-CoA | |
| IcmA, | isobutyryl-CoA mutase, | NP_629554 | 5.4.99.2 | express or | converting | Streptomyces coelicolor |
| isobutyryl-CoA | subunit A | | | Over-Express | butyryl-CoA to | |
| mutase | | | | | isobutyryl-CoA | |
| IcmA, | isobutyryl-CoA mutase, | AAC08713 | 5.4.99.2 | express or | converting | Streptomyces cinnamonensis |
| isobutyryl-CoA | subunit A | | | Over-Express | butyryl-CoA to | |
| mutase | | | | | isobutyryl-CoA | |
| IcmB, | isobutyryl-CoA mutase, | NP_630904 | 5.4.99.2 | express or | converting | Streptomyces coelicolor |
| isobutyryl-CoA | subunit B | | | Over-Express | butyryl-CoA to | |
| mutase | | | | | isobutyryl-CoA | |
| IcmB, | isobutyryl-CoA mutase, | CAB59633 | 5.4.99.2 | express or | converting | Streptomyces cinnamonensis |
| isobutyryl-CoA | subunit B | | | Over-Express | butyryl-CoA to | |
| mutase | | | | | isobutyryl-CoA | |
| FabH, ACPs and | | | | | | |
| fabF genes with | | | | | | |
| specificity for | | | | | | |
| branched chain | | | | | | |
| acyl-CoAs | | | | | | |
| IlvE | branched-chain amino | CAC12788 | EC2.6.1.42 | over express | branched chain | Staphylococcus carnosus |
| | acid aminotransferase | | | | amino acid amino | |
| | | | | | transferase | |
| FabH1 | beta-ketoacyl-ACP | NP_626634 | 2.3.1.180 | express or | initiation of | Streptomyces coelicolor |
| | synthase III | | | Over-Express | branched-chain | |
| | | | | | fatty acid | |
| | | | | | biosynthesis | |
| ACP | acyl-carrier protein | NP_626635 | NONE | express or | initiation and | Streptomyces coelicolor |
| | | | | Over-Express | elongation of | |
| | | | | | branched-chain | |
| | | | | | fatty acid | |
| | | | | | biosynthesis | |
| FabF | beta-ketoacyl-ACP | NP_626636 | 2.3.1.179 | express or | elongation of | Streptomyces coelicolor |
| | synthase II | | | Over-Express | branched-chain | |
| | | | | | fatty acid | |
| | | | | | biosynthesis | |
| FabH3 | beta-ketoacyl-ACP | NP_823466 | 2.3.1.180 | express or | initiation of | Streptomyces avermitilis |
| | synthase III | | | Over-Express | branched-chain | |
| | | | | | fatty acid | |
| | | | | | biosynthesis | |
| FabC3 (ACP) | acyl-carrier protein | NP_823467 | NONE | express or | initiation and | Streptomyces avermitilis |
| | | | | Over-Express | elongation of | |
| | | | | | branched-chain | |
| | | | | | fatty acid | |
| | | | | | biosynthesis | |
| FabF | beta-ketoacyl-ACP | NP_823468 | 2.3.1.179 | express or | elongation of | Streptomyces avermitilis |
| | synthase II | | | Over-Express | branched-chain | |
| | | | | | fatty acid | |
| | | | | | biosynthesis | |
| FabH_A | beta-ketoacyl-ACP | NP_389015 | 2.3.1.180 | express or | initiation of | Bacillus subtillis |
| | synthase III | | | Over-Express | branched-chain | |
| | | | | | fatty acid | |
| | | | | | biosynthesis | |
| FabH_B | beta-ketoacyl-ACP | NP_388898 | 2.3.1.180 | express or | initiation of | Bacillus subtillis |
| | synthase III | | | Over-Express | branched-chain | |
| | | | | | fatty acid | |
| | | | | | biosynthesis | |
| ACP | acyl-carrier protein | NP_389474 | NONE | express or | initiation and | Bacillus subtillis |
| | | | | Over-Express | elongation of | |
| | | | | | branched-chain | |
| | | | | | fatty acid | |
| | | | | | biosynthesis | |
| FabF | beta-ketoacyl-ACP | NP_389016 | 2.3.1.179 | express or | elongation of | Bacillus subtillis |
| | synthase II | | | Over-Express | branched-chain | |
| | | | | | fatty acid | |
| | | | | | biosynthesis | |
| SmalDRAFT_0818 | beta-ketoacyl-ACP | ZP_01643059 | 2.3.1.180 | express or | initiation of | Stenotrophomonas maltophilia |
| | synthase III | | | Over-Express | branched-chain | |
| | | | | | fatty acid | |
| | | | | | biosynthesis | |
| SmalDRAFT_0821 | acyl-carrier protein | ZP_01643063 | NONE | express or | initiation and | Stenotrophomonas maltophilia |
| | | | | Over-Express | elongation of | |
| | | | | | branched-chain | |
| | | | | | fatty acid | |
| | | | | | biosynthesis | |
| SmalDRAFT_0822 | beta-ketoacyl-ACP | ZP_01643064 | 2.3.1.179 | express or | elongation of | Stenotrophomonas maltophilia |
| | synthase II | | | Over-Express | branched-chain | |
| | | | | | fatty acid | |
| | | | | | biosynthesis | |
| FabH | beta-ketoacyl-ACP | YP_123672 | 2.3.1.180 | express or | initiation of | Legionella pneumophila |
| | synthase III | | | Over-Express | branched-chain | |
| | | | | | fatty acid | |
| | | | | | biosynthesis | |
| ACP | acyl-carrier protein | YP_123675 | NONE | express or | initiation and | Legionella pneumophila |
| | | | | Over-Express | elongation of | |
| | | | | | branched-chain | |
| | | | | | fatty acid | |
| | | | | | biosynthesis | |
| FabF | beta-ketoacyl-ACP | YP_123676 | 2.3.1.179 | express or | elongation of | Legionella pneumophila |
| | synthase II | | | Over-Express | branched-chain | |
| | | | | | fatty acid | |
| | | | | | biosynthesis | |
| FabH | beta-ketoacyl-ACP | NP_415609 | 2.3.1.180 | delete or reduce | initiation of | Escherichia coli |
| | synthase III | | | | branched-chain | |
| | | | | | fatty acid | |
| | | | | | biosynthesis | |
| FabF | beta-ketoacyl-ACP | NP_415613 | 2.3.1.179 | delete or reduce | elongation of | Escherichia coli |
| | synthase II | | | | branched-chain | |
| | | | | | fatty acid | |
| | | | | | biosynthesis | |

To Produce Cyclic Fatty Acids

| | | | | | | |
|---|---|---|---|---|---|---|
| AnsJ | dehydratase (putative) | not available | not | express or | cyclohexylcarbonyl- | Streptomyces collinus |
| | | | available | Over-Express | CoA | |
| | | | | | boiosynthesis | |
| AnsK | CoA ligase (putative) | not available | not | express or | cyclohexylcarbonyl- | Streptomyces collinus |
| | | | available | Over-Express | CoA | |
| | | | | | boiosynthesis | |
| AnsL | dehydrogenase (putative) | not available | not | express or | cyclohexylcarbonyl- | Streptomyces collinus |
| | | | available | Over-Express | CoA | |
| | | | | | boiosynthesis | |
| ChcA | enoyl-CoA reductase | U72144 | EC | express or | cyclohexylcarbonyl- | Streptomyces collinus |
| | | | 1.3.1.34 | Over-Express | CoA | |
| | | | | | boiosynthesis | |
| AnsM | oxidorecutase (putative) | not available | not | express or | cyclohexylcarbonyl- | Streptomyces collinus |
| | | | available | Over-Express | CoA | |
| | | | | | boiosynthesis | |
| PlmJ | dehydratase (putative) | AAQ84158 | not | express or | cyclohexylcarbonyl- | Streptomyces sp. HK803 |
| | | | available | Over-Express | CoA | |
| | | | | | boiosynthesis | |
| PlmK | CoA ligase (putative) | AAQ84158 | not | express or | cyclohexylcarbonyl- | Streptomyces sp. HK803 |
| | | | available | Over-Express | CoA | |
| | | | | | boiosynthesis | |
| PlmL | dehydrogenase (putative) | AAQ84159 | not | express or | cyclohexylcarbonyl- | Streptomyces sp. HK803 |
| | | | available | Over-Express | CoA | |
| | | | | | boiosynthesis | |
| ChcA | enoyl-CoA reductase | AAQ84160 | EC | express or | cyclohexylcarbonyl- | Streptomyces sp. HK803 |
| | | | 1.3.1.34 | Over-Express | CoA | |
| | | | | | boiosynthesis | |
| PlmM | oxidorecutase (putative) | AAQ84161 | not | express or | cyclohexylcarbonyl- | Streptomyces sp. HK803 |
| | | | available | Over-Express | CoA | |
| | | | | | boiosynthesis | |
| ChcB | enoyl-CoA isomerase | AF268489 | not | express or | cyclohexylcarbonyl- | Streptomyces collinus |
| | | | available | Over-Express | CoA | |
| | | | | | boiosynthesis | |
| ChcB/CaiD | enoyl-CoA isomerase | NP_629292 | 4.2.1.— | express or | cyclohexylcarbonyl- | Streptomyces coelicolor |
| | | | | Over-Express | CoA | |
| | | | | | boiosynthesis | |
| ChcB/CaiD | enoyl-CoA isomerase | NP_824296 | 4.2.1.— | express or | cyclohexylcarbonyl- | Streptomyces avermitilis |
| | | | | Over-Express | CoA | |
| | | | | | boiosynthesis | |

2C. Saturation Level Control

| | | | | | | |
|---|---|---|---|---|---|---|
| Sfa | Suppressor of FabA | AAN79592, | NONE | Over-express | increase | Ecoli |
| | | AAC44390 | | | monounsaturated | |
| | | | | | fatty acids | |
| also see FabA in | | | | express | produce | |
| sec. 1 | | | | | unsaturated fatty | |
| | | | | | acids | |
| GnsA | suppressors of the secG | ABD18647.1 | NONE | Over-express | increase | E. coli |
| | null mutation | | | | unsaturated fatty | |
| | | | | | acid esters | |
| GnsB | suppressors of the secG | AAC74076.1 | NONE | Over-express | increase | E. coli |
| also see section | null mutation | | | | unsaturated fatty | |
| 2A - items with: 0 | | | | | acid esters | |
| are unsaturated | | | | | | |
| (no double bonds) | | | | | | |
| and with: 1 are | | | | | | |
| saturated (1 | | | | | | |
| double bond) | | | | | | |
| fabB | 3-oxoacyl-[acyl-carrier-protein] | BAA16180 | EC: 2.3.1.41 | overexpress | modulate | Escherichia coli |
| | synthase I | | | | unsaturated fatty | |
| | | | | | acid production | |
| fabK | trans-2-enoyl-ACP | AAF98273 | 1.3.1.9 | express | modulate | Streptococcus pneumoniae |
| | reductase II | | | | unsaturated fatty | |
| | | | | | acid production | |
| fabL | enoyl-(acyl carrier | AAU39821 | 1.3.1.9 | express | modulate | Bacillus licheniformis DSM 13 |
| | protein) reductase | | | | unsaturated fatty | |
| | | | | | acid production | |
| fabM | trans-2, | DAA05501 | 4.2.1.17 | Over-express | modulate | Streptococcus mutans |
| | cis-3-decenoyl-ACP | | | | unsaturated fatty | |
| | isomerase | | | | acid production | |

Fatty Aldehyde Output

| | | | |
|---|---|---|---|
| Thioesterase | see chain length control | express | produce |
| | section | | |

Export

| | | | | | | |
|---|---|---|---|---|---|---|
| Wax ester exporter | | NP_524723 | NONE | express | export wax | Drosophila melanogaster |
| (FATP family, | | | | | | |
| Fatty Acid (long | | | | | | |
| chain) Transport | | | | | | |
| Protein) | | | | | | |
| ABC transport | putative alkane transporter | AAN73268 | NONE | express | export products | Rhodococcus erythropolis |
| protein | | | | | | |
| CER5 | wax transporter | At1g51500, | NONE | express | export products | Arabidopsis thaliana |
| | | AY734542, | | | | |
| | | At3g21090, | | | | |
| | | At1g51460 | | | | |
| AtMRP5 | Arabidopsis thaliana | NP_171908 | NONE | express | export products | Arabidopsis thaliana |
| | multidrug | | | | | |
| | resistance-associated | | | | | |
| AmiS2 | ABC transporter AmiS2 | JC5491 | NONE | express | export products | Rhodococcus sp. |
| AtPGP1 | ARABIDOPSIS | NP_181228 | NONE | express | export products | Arabidopsis thaliana |
| | THALIANA P | | | | | |
| | GLYCOPROTEIN1 | | | | | |
| AcrA | putative multidrug-efflux | CAF23274 | NONE | express | export products | Candidatus Protochlamydia amoebophila UWE25 |
| | transport protein acrA | | | | | |
| AcrB | probable multidrug-efflux | CAF23275 | NONE | express | export products | Candidatus Protochlamydia amoebophila UWE25 |
| | transport protein, acrB | | | | | |
| TolC | Outer membrane protein | ABD59001 | NONE | express | export products | Francisella tularensis subsp. Novicida |
| | [Cell envelope biogenesis, | | | | | |
| AcrE | transmembrane protein | YP_312213 | NONE | express | export products | Shigella sonnei Ss046 |
| | affects septum formation | | | | | |
| | and cell membrane | | | | | |
| | permeability | | | | | |
| AcrF | Acriflavine resistance | P24181 | NONE | express | export products | Escherichia coli |
| | protein F | | | | | |
| tll1618 | multidrug efflux | NP_682408.1 | NONE | express | export products | Thermosynechococcus elongatus BP-1] |
| | transporter | | | | | |
| tll1619 | multidrug efflux | NP_682409.1 | NONE | express | export products | Thermosynechococcus elongatus BP-1] |
| | transporter | | | | | |
| tll0139 | multidrug efflux | NP_680930.1 | NONE | express | export products | Thermosynechococcus elongatus BP-1] |
| | transporter | | | | | |

5. Fermentation

| | | | | | | |
|---|---|---|---|---|---|---|
| Replication | | | | | increase output | |
| checkpoint genes | | | | | efficiency | |
| umuD | DNA polymerase V, | YP_310132 | 3.4.21.— | Over-express | increase output | Shigella sonnei Ss046 |
| | subunit | | | | efficiency | |
| umuC | DNA polymerase V, | ABC42261 | 2.7.7.7 | Over-express | increase output | Escherichia coli |
| | subunit | | | | efficiency | |
| NADH:NADPH | | P07001, | 1.6.1.2 | express | increase output | Shigella flexneri |
| transhydrogenase | | P0AB70 | | | efficiency | |
| (alpha and beta | | | | | | |
| subunits) (pntA, | | | | | | |
| pntB) | | | | | | |

### Synthesis of Fatty Alcohols and Substrates

Fatty acid synthase (FAS) is a group of polypeptides that catalyze the initiation and elongation of acyl chains (Marrakchi et al., Biochemical Society. 30: 1050-1055 (2002)). The acyl carrier protein (ACP) along with the enzymes in the FAS pathway control the length, degree of saturation, and branching of the fatty acid derivatives produced. The fatty acid biosynthetic pathway involves the precursors acetyl-CoA and malonyl-CoA. The steps in this pathway are catalyzed by enzymes of the fatty acid biosynthesis (fab) and acetyl-CoA carboxylase (acc) gene families (see, e.g., Heath et al., Prog. Lipid Res., 40(6): 467-97 (2001)).

Host cells can be engineered to express fatty acid derivative substrates by recombinantly expressing or overexpressing one or more fatty acid synthase genes, such as acetyl-CoA and/or malonyl-CoA synthase genes. For example, to increase acetyl-CoA production, one or more of the following genes can be expressed in a host cell: pdh (a multienzyme complex comprising aceEF (which encodes the E1p dehydrogenase component, the E2p dihydrolipoamide acyltransferase component of the pyruvate and 2-oxoglutarate dehydrogenase complexes, and lpd), panK, fabH, fabB, fabD, fabG, acpP, and fabF. Exemplary GenBank accession numbers for these genes are: pdh (BAB34380, AAC73227, AAC73226), panK (also known as CoA, AAC76952), aceEF (AAC73227, AAC73226), fabH (AAC74175), fabB (P0A953), fabD (AAC74176), fabG (AAC74177), acpP (AAC74178), and fabF (AAC74179). Additionally, the expression levels of fadE, gpsA, ldhA, pflb, adhE, pta, poxB, ackA, and/or ackB can be attenuated or knocked-out in an engineered host cell by transformation with conditionally replicative or non-replicative plasmids containing null or deletion mutations of the corresponding genes or by substituting promoter or enhancer sequences. Exemplary GenBank accession numbers for these genes are: fadE (AAC73325), gspA (AAC76632), ldhA (AAC74462), pflb (AAC73989), adhE (AAC74323), pta (AAC75357), poxB (AAC73958), ackA (AAC75356), and ackB (BAB81430). The resulting host cells will have increased acetyl-CoA production levels when grown in an appropriate environment.

Malonyl-CoA overexpression can be affected by introducing accABCD (e.g., accession number AAC73296, EC 6.4.1.2) into a host cell. Fatty acid production can be further increased by introducing into the host cell a DNA sequence encoding a lipase (e.g., accession numbers CAA89087, CAA98876).

In addition, inhibiting PlsB can lead to an increase in the levels of long chain acyl-ACP, which will inhibit early steps in the pathway (e.g., accABCD, fabH, and fabI). The plsB (e.g., accession number AAC77011) D311E mutation can be used to increase the amount of available fatty acids.

In addition, a host cell can be engineered to overexpress a sfa gene (suppressor of fabA, e.g., accession number AAN79592) to increase production of monounsaturated fatty acids (Rock et al., J. Bacteriology, 178: 5382-5387 (1996)).

The chain length of a fatty acid derivative substrate can be selected for by modifying the expression of selected thioesterases. Thioesterase influences the chain length of fatty acids produced. Hence, host cells can be engineered to express, overexpress, have attenuated expression, or not to express one or more selected thioesterases to increase the production of a preferred fatty acid derivative substrate. For example, C₁₀ fatty acids can be produced by expressing a thioesterase that has a preference for producing C₁₀ fatty acids and attenuating thioesterases that have a preference for producing fatty acids other than C₁₀ fatty acids (e.g., a thioesterase which prefers to produce C₁₄ fatty acids). This would result in a relatively homogeneous population of fatty acids that have a carbon chain length of 10. In other instances, C₁₄ fatty acids can be produced by attenuating endogenous thioesterases that produce non-C₁₄ fatty acids and expressing the thioesterases that have a preference for C₁₄-ACP. In some situations, C₁₂ fatty acids can be produced by expressing thioesterases that have a preference for C₁₂-ACP and attenuating thioesterases that preferentially produce non-C₁₂ fatty acids. Acetyl-CoA, malonyl-CoA, and fatty acid overproduction can be verified using methods known in the art, for example, by using radioactive precursors, HPLC, or GC-MS subsequent to cell lysis, Non-limiting examples of thioesterases that can be used in the methods described herein are listed in Table 1.

TABLE 1
Thioesterases

| Accession Number | Source Organism | Gene |
|---|---|---|
| AAC73596 | E. coli | tesA without |
| | | leader sequence |
| AAC73555 | E. coli | tesB |
| Q41635, AAA34215 | Umbellurlaria california | fatB |
| AAC49269 | Cuphea hookeriana | fatB2 |
| Q39513; AAC72881 | Cuphea hookeriana | fatB3 |
| Q39473, AAC49151 | Cinnamonum camphorum | fatB |
| CAA85388 | Arabisopsis thaliana | fatB [M141T]* |
| NP 189147; NP 193401 | Arabidopsis thaliana | fatA |
| CAC39106 | Bradyrhiizobium japonicum | fatA |
| AAC72883 | Cuphea hookeriana | fatA |
| AAL79361 | Helianthus annus | fatA1 |

*Mayer et al., BMC Plant Biology, 7: 1-11 (2007)

In other instances, a fatty aldehyde biosynthetic polypeptide, variant, or a fragment thereof is expressed in a host cell that contains a naturally occurring mutation that results in an increased level of fatty acids in the host cell. In some instances, the host cell is genetically engineered to increase the level of fatty acids in the host cell relative to a corresponding wild-type host cell. For example, the host cell can be genetically engineered to express a reduced level of an acyl-CoA synthase relative to a corresponding wild-type host cell. In one embodiment, the level of expression of one or more genes (e.g., an acyl-CoA synthase gene) is reduced by genetically engineering a “knock out” host cell.

Any known acyl-CoA synthase gene can be reduced or knocked out in a host cell. Non-limiting examples of acyl-CoA synthase genes include fadD, fadK, BH3103, yhfL, Pfl-4354, EAV15023, fadD1, fadD2, RPC_{—}4074, fadDD35, fadDD22, faa3p or the gene encoding the protein ZP_{—}01644857. Specific examples of acyl-CoA synthase genes include fadDD35 from M. tuberculosis H37Rv [NP_{—}217021], fadK from M. tuberculosis H37Rv [NP_{—}217464], fadD from E. coli [NP_{—}217464], fadK from E. coli [NP_{—}416319], fadK from E. coli [YP_{—}416216], fadD from Acinetobacter sp. ADP1 [YP_{—}045024], fadD from Haemophilus influenza RdkW20 [NP_{—}43855], POD from Rhodopseudomonas palustris Bis B18 [YP_{—}533919], BH3101 from Bacillus halodurans C-125 [NP_{—}243969], Pfl-4354 from Pseudomonas fluorescens Pfo-1 [YP_{—}350082], EAV15023 from Comamonas testosterone KF-1 [ZP_{—}01520072], yhfL from B. subtilis [NP_{—}388908], fadD1 from P. aeruginosa PAO1 [NP_{—}251989], fadD1 from Ralstonia solanacearum GM1 1000 [NP_{—}520978], fadD2 from P. aeruginosa PAO1 [NP_{—}251990], the gene encoding the protein ZP_{—}01644857 from Stenotrophomonas maltophilia R551-3, faa3p from Saccharomyces cerevisiae [NP_{—}012257], faa1p from Saccharomyces cerevisiae [NP_{—}014962], lcfA from Bacillus subtilis [CAA99571], or those described in Shockey et al., Plant. Physiol., 129: 1710-1722 (2002); Caviglia et al., J. Biol. Chem., 279: 1163-1169 (2004); Knoll et al., J. Biol. Chem., 269(23): 16348-56 (1994); Johnson et al., J. Biol. Chem., 269: 18037-18046 (1994); and Black et al., J. Biol. Chem. 267: 25513-25520 (1992).

### Formation of Branched Substrates and Fatty Alcohols

Fatty alcohols can be produced from fatty aldehydes that contain branch points by using branched fatty acid derivatives as substrates for a fatty aldehyde biosynthetic polypeptide described herein. For example, although E. coli naturally produces straight chain fatty acids (sFAs), E. coli can be engineered to produce branched chain fatty acids (brFAs) by introducing and expressing or overexpressing genes that provide branched precursors in the E. coli (e.g., by expressing genes from the following gene families: bkd, ilv, icm, and fab). Additionally, a host cell can be engineered to express or overexpress genes encoding proteins for the initiation (e.g., FabH) and elongation of brFAs (e.g. ACP, FabF, etc.) and/or to delete or attenuate the corresponding host cell genes that normally lead to sFAs.

The first step in forming brFAs is the production of the corresponding α-keto acids by a branched-chain amino acid aminotransferase. Host cells may endogenously include genes encoding such enzymes or such genes can be recombinantly introduced. E. coli, for example, endogenously expresses such an enzyme, IlvE (EC 2.6.1.42; GenBank accession YP_{—}026247). In some host cells, a heterologous branched-chain amino acid aminotransferase may not be expressed. However, E. coli IlvE or any other branched-chain amino acid aminotransferase IlvE from Lactococcus lactis (GenBank accession AAF34406), IlvE from Pseudomonas putida (GenBank accession NP_{—}745648), or IlvE from Streptornyces coelicolor (GenBank accession NP_{—}629657)), if not endogenous, can be introduced.

In another embodiment, the production of α-keto acids can be achieved by using the methods described in Park et al., PNAS, 104:7797-7802 (2007) and Atsumi et al., Nature, 451: 86-89 (2008). For example, 2-ketoisovalerate can be produced by overexpressing the genes encoding IlvI, IlvH, IlvH mutant, IlvB, IlvN, IlvGM, IlvC, or IlvD. In another example, 2-keto-3-methyl-valerate can be produced by overexpressing the genes encoding IlvA and IlvI, IlvH (or AlsS of Bacillus subtilis), IlvC, IlvD, or their corresponding homologs. In a further embodiment, 2-keto-4-methyl-pentanoate can be produced by overexpressing the genes encoding IlvI, IlvH, IlvC, IlvD and LeuA, LeuB, LeuC, LeuD, or their corresponding homologs.

The second step is the oxidative decarboxylation of the α-keto acids to the corresponding branched-chain acyl-CoA. This reaction can be catalyzed by a branched-chain α-keto acid dehydrogenase complex (bkd; EC 1.2.4.4.) (Denoya et al., J. Bacterial., 177: 3504 (1995)), which consists of E1α/β (decarboxylase), E2 (dihydrolipoyl transacylase), and E3 (dihydrolipoyl dehydrogenase) subunits. These branched-chain α-keto acid dehydrogenase complexes are similar to pyruvate dehydrogenase complexes and α-ketoglutarate dehydrogenase complexes. Any microorganism that possesses brFAs and/or grows on branched-chain amino acids can be used as a source to isolate bkd genes for expression in host cells, for example, E. coli. Furthermore, E. coli has the E3 component as part of its pyruvate dehydrogenase complex (lpd, EC 1.8.1.4, GenBank accession NP_{—}414658). Thus, it may be sufficient to express only the E1 α/β and E2 bkd genes. Table 2 lists non-limiting examples of bkd genes from several microorganisms that can be recombinantly introduced and expressed in a host cell to provide branched-chain acyl-CoA precursors.

TABLE 2
Bkd Genes from Selected Microorganisms

| Organism | Gene | GenBank Accession # |
|---|---|---|
| Streptomyces coelicolor | bdkA1 (E1α) | NP_628006 |
| | bdkB1 (E1β) | NP_628005 |
| | bkdC1 (E2) | NP_638004 |
| Streptomyces coelicolor | bdkA2 (E1α) | NP_733618 |
| | bdkB2 (E1β) | NP_628019 |
| | bkdC2 (E2) | NP_628018 |
| Streptomyces avermitilis | bdkA (E1a) | BAC72074 |
| | bdkB (E1b) | BAC72075 |
| | bkdC (E2) | BAC72076 |
| Streptomyces avermitilis | bdkF (E1α) | BAC72088 |
| | bdkG (E1β) | BAC72089 |
| | bkdH (E2) | BAC72090 |
| Bacillus subtilis | bdkAA (E1α) | NP_390285 |
| | bdkAB (E1β) | NP_390284 |
| | bkdB (E2) | NP_390382 |
| Pseudomonas putida | bdkA1 (E1α) | AAA65614 |
| | bdkA2 (E1β) | AAA65615 |
| | bkdC (E2) | AAA65617 |

In another example, isobutyryl-CoA can be made in a host cell, for example in E. coli, through the coexpression of a crotonyl-CoA reductase (Ccr, EC 1.6.5.5, 1.1.1.1) and isobutyryl-CoA mutase (large subunit IcmA, EC 5.4.99.2; small subunit IcmB, EC 5.4.99.2) (Han and Reynolds, J. Bacteriol., 179: 5157 (1997)). Crotonyl-CoA is an intermediate in fatty acid biosynthesis in E. coli and other microorganisms. Non-limiting examples of ccr and icm genes from selected microorganisms are listed in Table 3,

TABLE 3
ccr and icm Genes from Selected Microorganisms

| Organism | Gene | GenBank Accession # |
|---|---|---|
| Streptomyces | Ccr | NP_630556 |
| coelicolor | icmA | NP_629554 |
| | icmB | NP_630904 |
| Streptomyces | Ccr | AAD53915 |
| cinnamonensis | icmA | AAC08713 |
| | icmB | AJ246005 |

In addition to expression of the bkd genes, a β-ketoacyl-acyl-carrier-protein synthase III (FabH, EC 2.3.1.41) with preferred specificity for branched chain acyl-CoAs (Li et al., J. Bacteriol., 187: 3795-3799 (2005) can be heteroglogously overexpressed to increase brFabA biosynthesis. Non-limiting examples of such FabH enzymes are listed in Table 4. fabH genes that are involved in fatty acid biosynthesis of any brFA-containing microorganism can be expressed in a host cell. The Bkd and FabH enzymes from host cells that do not naturally make brFA may not support brFA production. Therefore, bkd and fabH can be expressed recombinantly. Vectors containing the bkd and fabH genes can be inserted into such a host cell. Similarly, the endogenous level of Bkd and FabH production may not be sufficient to produce brFA. In this case, they can be overexpressed. Additionally, other components of the fatty acid biosynthesis pathway can be expressed or overexpressed, such as acyl carrier proteins (ACPs) and β-ketoacyl-acyl-carrier-protein synthase II (fabF, EC 2.3.1.41) (non-limiting examples of candidates are listed in Table 4). In addition to expressing these genes, some genes in the endogenous fatty acid biosynthesis pathway can be attenuated in the host cell (e.g., the E. coli genes fabH (GenBank accession # NP_{—}415609) and/or fabF (GenBank accession # NP_{—}415613)).

TABLE 4
FabH, ACP and fabF Genes from Selected
Microorganisms with brFAs

| Organism | Gene | GenBank Accession # |
|---|---|---|
| Streptomyces | fabH1 | NP_626634 |
| coelicolor | acp | NP_626635 |
| | fabE | NP_626636 |
| Streptomyces | fabH3 | NP_823466 |
| avermitilis | fabC3 (acp) | NP_823467 |
| | fabF | NP_823468 |
| Bacillus subtilis | fabH_A | NP_389015 |
| | fabH_B | NP_388898 |
| | acp | NP_389474 |
| | fabF | NP_389016 |
| Stenotrophomonas | SmalDRAFT_0818 (fabH) | ZP_01643059 |
| maltophilia | SmalDRAFT_0821 (acp) | ZP_01643063 |
| | SmalDRAFT_0822 (fabF) | ZP_01643064 |
| Legionella | fabH | YP_123672 |
| pneumophila | acp | YP_123675 |
| | fabF | YP_123676 |

### Formation of Cyclic Substrates and Fatty Alcohols

Cyclic fatty alcohols can be produced from cyclic fatty aldehydes using cyclic fatty acid derivatives as substrates for a fatty aldehyde biosynthetic polypeptide described herein. To produce cyclic fatty acid derivative substrates, genes that provide cyclic precursors (e.g., the ans, chc, and plm gene families, see Table 5) can be introduced into the host cell and expressed to allow initiation of fatty acid biosynthesis from cyclic precursors. For example, to convert a host cell, such as E. coli, into one capable of synthesizing ω-cyclic fatty acids (cyFA), a gene that provides the cyclic precursor cyclohexylcarbonyl-CoA (CHC-CoA) (Cropp et al., Nature Biotech., 18: 980-983 (2000)) can be introduced and expressed in the host cell. Non-limiting examples of genes that provide CHC-CoA in E. coli include: ansJ, ansK, ansL, chcA, and ansM from the ansatrienin gene cluster of Streptomyces collinus (Chen et al., Eur. J. Biochem., 26): 98-107 (1999)) or plmJ, plmK, plmL, chcA, and plmM from the phoslactomycin B gene cluster of Streptomyces sp. HK803 (Palaniappan et al., J. Biol. Chem., 278: 35552-35557 (2003)) together with the chcB gene (Patton et al., Biochem., 39: 7595-7604 (2000)) from S. collinus, S. avermitilis, or S. coelicolor (see Table 5). The genes listed in Table 4 can then be expressed to allow initiation and elongation of ω-cyclic fatty acids. Alternatively, the homologous genes can be isolated from microorganisms that make cyFA and expressed in a host cell (e.g., E. coli).

TABLE 5
Genes for the Synthesis of CHC-CoA

| Organism | Gene | GenBank Accession # |
|---|---|---|
| Streptomyces collinus | ansJK | U72144* |
| | ansL | |
| | chcA | |
| | ansM | |
| | chcB | AF268489 |
| Streptomyces sp. HK803 | pmlJK | AAQ84158 |
| | pmlL | AAQ84159 |
| | chcA | AAQ84160 |
| | pmlM | AAQ84161 |
| Streptomyces coelicolor | orchcB/caiD | NP_629292 |
| Streptomyces avermitilis | chcB/caiD | NP_629292 |

*Only chcA is annotated in GenBank entry U72144; ansJKLM are according to Chen et al. (Eur. J. Biochem., 261: 98-107 (1999)).

The genes listed in Table 4 (fabH, acp, and fabF) allow initiation and elongation of ω-cyclic fatty acids because they have broad substrate specificity. If the coexpression of any of these genes with the genes listed in Table 5 does not yield cyFA, then fabH, acp, and/or fabF homologs from microorganisms that make cyFAs (e.g., those listed in Table 6) can be isolated (e.g., by using degenerate PCR primers or heterologous DNA sequence probes) and coexpressed.

TABLE 6
Non-Limiting Examples of Microorganisms
that Contain ω-cyclic Fatty Acids

| Organism | Reference |
|---|---|
| Curtobacterium pusillum | ATCC19096 |
| Alicyclobacillus acidoterrestris | ATCC49025 |
| Alicyclobacillus acidocaldarius | ATCC27009 |
| Alicyclobacillus cycloheptanicus* | Moore, J. Org. |
| | Chem, 62: 2173 (1997) |

*Uses cycloheptylcarbonyl-CoA and not cyclohexylcarbonyl-CoA as precursor for cyFA biosynthesis.

### Substrate and Fatty Alcohol Saturation Levels

The degree of saturation in fatty acids (which can then be converted into fatty aldehydes and then fatty alcohols as described herein) can be controlled by regulating the degree of saturation of fatty acid intermediates. The sfa, gns, and fab families of genes can be expressed or overexpressed to control the saturation of fatty acids. Table A lists non-limiting examples of genes in these gene families that may be used in the methods and host cells described herein.

Host cells can be engineered to produce unsaturated fatty acids by engineering the production host to overexpress fabB or by growing the production host at low temperatures (e.g., less than 37° C.). FabB has preference to cis-δ3decenoyl-ACP and results in unsaturated fatty acid production in E. coli. Overexpression of fabB results in the production of a significant percentage of unsaturated fatty acids (de Mendoza et al., J. Biol. Chem., 258: 2098-2101 (1983)). The gene fabB may be inserted into and expressed in host cells not naturally having the gene. These unsaturated fatty acids can then be used as intermediates in host cells that are engineered to produce fatty acid derivatives, such as fatty aldehydes.

In other instances, a repressor of fatty acid biosynthesis, for example, fabR (GenBank accession NP_{—}418398), can be deleted, which will also result in increased unsaturated fatty acid production in E. coli (Zhang et al., J. Biol. Chem., 277: 15558 (2002)). Similar deletions may be made in other host cells. A further increase in unsaturated fatty acids may be achieved, for example, by overexpressing fabM (trans-2, cis-3-decenoyl-ACP isomerase. GenBank accession DAA05501) and controlled expression of fabK (trans-2-enoyl-ACP reductase II, GenBank accession NP_{—}357969) from Streptococcus pneumoniae (Marrakchi et al., J. Biol. Chem., 277: 44809 (2002)), while deleting E. coli fabI (trans-2-enoyl-ACP reductase, GenBank accession NP_{—}415804). In some examples, the endogenous fabF gene can be attenuated, thus increasing the percentage of palmitoleate (C16:1) produced.

### Fatty Aldehyde Biosynthetic Polynucleotides and Variants

The methods described herein can be used to produce fatty alcohols, for example, from fatty aldehydes. In some instances, a fatty aldehyde is produced by expressing a fatty aldehyde biosynthetic gene, for example, a carboxylic acid reductase gene (car gene), having a nucleotide sequence listed in Sequence Listings 1 and 2, as well as polynucleotide variants thereof. In some instances, the fatty aldehyde biosynthetic gene encodes one or more of the amino acid motifs depicted in “Amino Acid Sequence Motifs 1”. For example, the gene can encode a polypeptide comprising (a) SEQ ID NO:7, SEQ ID NO:8, SEQ ID NO:9, and SEQ ID NO:10; (b) SEQ ID NO:11; (c) SEQ ID NO:12; (d) SEQ ID NO:13; (e) SEQ ID NO:14; and/or (f) SEQ ID NO:7, SEQ ID NO:9, SEQ ID NO:10, and SEQ ID NO:11. SEQ ID NO:7 includes a reductase domain; SEQ ID NO:8 and SEQ ID NO:14 include a NADP binding domain; SEQ ID NO:9 includes a phosphopantetheine attachment site; and SEQ ID NO:10 includes an AMP binding domain.

Any polynucleotide sequence encoding a homolog listed in Sequence Listings 1 and 2, or a variant thereof, can be used as a fatty aldehyde biosynthetic polynucleotide in the methods described herein.

### Production of Genetic Variants

Variants can be naturally occurring or created in vitro. In particular, such variants can be created using genetic engineering techniques, such as site directed mutagenesis, random chemical mutagenesis, Exonuclease III deletion procedures, or standard cloning techniques. Alternatively, such variants, fragments, analogs, or derivatives can be created using chemical synthesis or modification procedures.

Methods of making variants are well known in the art. These include procedures in which nucleic acid sequences obtained from natural isolates are modified to generate nucleic acids that encode polypeptides having characteristics that enhance their value in industrial or laboratory applications. In such procedures, a large number of variant sequences having one or more nucleotide differences with respect to the sequence obtained from the natural isolate are generated and characterized. Typically, these nucleotide differences result in amino acid changes with respect to the polypeptides encoded by the nucleic acids from the natural isolates.

For example, variants can be created using error prone PCR (see, e.g., Leung et al., Technique, 1: 11-15 (1989); and Caldwell et al., PCR Methods Applic., 2: 28-33 (1992)). In error prone PCR, PCR is performed under conditions where the copying fidelity of the DNA polymerase is low, such that a high rate of point mutations is obtained along the entire length of the PCR product. Briefly, in such procedures, nucleic acids to be mutagenized (e.g., a fatty aldehyde biosynthetic polynucleotide sequence) are mixed with PCR primers, reaction buffer, MgCl₂, MnCl₂, Taq polymerase, and an appropriate concentration of dNTPs for achieving a high rate of point mutation along the entire length of the PCR product. For example, the reaction can be performed using 20 fmoles of nucleic acid to be mutagenized (e.g., a fatty aldehyde biosynthetic polynucleotide sequence), 30 μmole of each PCR primer, a reaction buffer comprising 50 mM KCl, 10 mM Tris HCl (pH 8.3), and 0.01% gelatin, 7 mM MgCl₂, 0.5 mM MnCl₂, 5 units of Taq polymerase, 0.2 mM dGTP, 0.2 mM dATP, 1 mM dCTP, and 1 mM dTTP. PCR can be performed for 30 cycles of 94° C. for 1 min, 45° C. for 1 min, and 72° C. for 1 min. However, it will be appreciated that these parameters can be varied as appropriate. The mutagenized nucleic acids are then cloned into an appropriate vector and the activities of the polypeptides encoded by the mutagenized nucleic acids are evaluated.

Variants can also be created using oligonucleotide directed mutagenesis to generate site-specific mutations in any cloned DNA of interest. Oligonucleotide mutagenesis is described in, for example, Reidhaar-Olson et al., Science, 241: 53-57 (1988). Briefly, in such procedures a plurality of double stranded oligonucleotides bearing one or more mutations to be introduced into the cloned DNA are synthesized and inserted into the cloned DNA to be mutagenized (e.g., a fatty aldehyde biosynthetic polynucleotide sequence). Clones containing the mutagenized DNA are recovered, and the activities of the polypeptides they encode are assessed.

Another method for generating variants is assembly PCR. Assembly PCR involves the assembly of a PCR product from a mixture of small DNA fragments. A large number of different PCR reactions occur in parallel in the same vial, with the products of one reaction priming the products of another reaction. Assembly PCR is described in, for example, U.S. Pat. No. 5,965,408.

Still another method of generating variants is sexual PCR mutagenesis. In sexual PCR mutagenesis, forced homologous recombination occurs between DNA molecules of different, but highly related, DNA sequence in vitro as a result of random fragmentation of the DNA molecule based on sequence homology. This is followed by fixation of the crossover by primer extension in a PCR reaction. Sexual PCR mutagenesis is described in, for example, Stemmer, Proc. Natl. Acad. Sci. USA, 91: 10747-10751 (1994).

Variants can also be created by in vivo mutagenesis. In some embodiments, random mutations in a nucleic acid sequence are generated by propagating the sequence in a bacterial strain, such as an E. coli strain, which carries mutations in one or more of the DNA repair pathways. Such “mutator” strains have a higher random mutation rate than that of a wild-type strain. Propagating a DNA sequence (e.g., a fatty aldehyde or fatty alcohol biosynthetic polynucleotide sequence) in one of these strains will eventually generate random mutations within the DNA. Mutator strains suitable for use for in vivo mutagenesis are described in, for example, PCT Publication No. WO 91/016427.

Variants can also be generated using cassette mutagenesis. In cassette mutagenesis, a small region of a double stranded DNA molecule is replaced with a synthetic oligonucleotide “cassette” that differs from the native sequence. The oligonucleotide often contains a completely and/or partially randomized native sequence.

Recursive ensemble mutagenesis can also be used to generate variants. Recursive ensemble mutagenesis is an algorithm for protein engineering (i.e., protein mutagenesis) developed to produce diverse populations of phenotypically related mutants whose members differ in amino acid sequence. This method uses a feedback mechanism to control successive rounds of combinatorial cassette mutagenesis. Recursive ensemble mutagenesis is described in, for example, Arkin et al., Proc. Natl. Acad. Sci. USA, 89: 7811-7815 (1992).

In some embodiments, variants are created using exponential ensemble mutagenesis. Exponential ensemble mutagenesis is a process for generating combinatorial libraries with a high percentage of unique and functional mutants, wherein small groups of residues are randomized in parallel to identify, at each altered position, amino acids which lead to functional proteins. Exponential ensemble mutagenesis is described in, for example, Delegrave et al., Biotech. Res., 11: 1548-1552 (1993). Random and site-directed mutagenesis are described in, for example, Arnold, Curr. Opin. Biotech., 4: 450-455 (1993).

In some embodiments, variants are created using shuffling procedures wherein portions of a plurality of nucleic acids that encode distinct polypeptides are fused together to create chimeric nucleic acid sequences that encode chimeric polypeptides as described in, for example, U.S. Pat. Nos. 5,965,408 and 5,939,250.

Polynucleotide variants also include nucleic acid analogs. Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine and 5-methyl-2′-deoxycytidine or 5-bromo-2′-doxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six-membered, morpholino ring, or peptide nucleic acids, in which the deoxyphosphate backbone is replaced by a pseudopeptide backbone and the four bases are retained. (See, e.g., Summerton et al., Antisense Nucleic Acid Drug Dev., 7: 187-195 (1997); and Hyrup et al., Bioorgan. Med. Chem., 4: 5-23 (1996).) In addition, the deoxyphosphate backbone can be replaced with, for example, a phosphorothioate or phosphorodithioate backbone, a phosphoroamidite, or an alkyl phosphotriester backbone.

### Fatty Aldehyde Biosynthetic Polypeptides and Variants

The methods described herein can also be used to produce fatty alcohols, for example, from fatty aldehydes. In some instances, the fatty aldehyde is produced by a fatty aldehyde biosynthetic polypeptide having an amino acid sequence listed in Sequence Listings 1 and 2, as well as polypeptide variants thereof. In some instances, a fatty aldehyde biosynthetic polypeptide is one that includes one or more of the amino acid motifs depicted in “Amino Acid Sequence Motifs 1”. For example, the polypeptide can include the amino acid sequences of SEQ ID NO:7, SEQ ID NO:8, SEQ ID NO:9, and SEQ ID NO:10. In other situations, the polypeptide includes one or more of SEQ ID NO:11, SEQ ID NO:12, SEQ ID NO:13, and SEQ ID NO:14. In yet other instances, the polypeptide includes the amino acid sequences of SEQ ID NO:7, SEQ ID NO:9, SEQ ID NO:10, and SEQ ID NO:11. SEQ ID NO:7 includes a reductase domain: SEQ ID NO:8 and SEQ ID NO:14 include a NADP binding domain: SEQ ID NO:9 includes a phosphopantetheine attachment site: and SEQ ID NO:10 includes an AMP binding domain. Fatty aldehyde biosynthetic polypeptide variants can be variants in which one or more amino acid residues are substituted with a conserved or non-conserved amino acid residue (preferably a conserved amino acid residue). Such substituted amino acid residue may or may not be one encoded by the genetic code.

In some instances, the polypeptide variants described herein retain the same biological function as a polypeptide having an amino acid sequence listed in Sequence Listings 1 and 2 (e.g., retain fatty aldehyde biosynthetic activity, such as carboxylic acid or fatty acid reductase activity) and have amino acid sequences substantially identical thereto.

In other instances, the polypeptide variants have at least about 50%, at least about 55%, at least about 60%, at least about 65%, at least about 70%, at least about 75%, at least about 80%, at least about 85%, at least about 90%, at least about 95%, or more than about 95% homology to an amino acid sequence listed in Sequence Listings 1 and 2. In another embodiment, the polypeptide variants include a fragment comprising at least about 5, 10, 15, 20, 25, 30, 35, 40, 50, 75, 100, or 150 consecutive amino acids thereof.

The sequence of the polypeptide variants or fragments can then be compared to an amino acid sequence listed in Sequence Listings 1 and 2 using any of the programs described herein.

### Production of Polypeptide Variants

Conservative substitutions are those that substitute a given amino acid in a polypeptide by another amino acid of similar characteristics. Typical conservative substitutions are the following replacements: replacement of an aliphatic amino acid, such as alanine, valine, leucine, and isoleucine, with another aliphatic amino acid; replacement of a serine with a threonine or vice versa; replacement of an acidic residue, such as aspartic acid and glutamic acid, with another acidic residue; replacement of a residue bearing an amide group, such as asparagine and glutamine, with another residue bearing an amide group; exchange of a basic residue, such as lysine and arginine, with another basic residue; and replacement of an aromatic residue, such as phenylalanine and tyrosine, with another aromatic residue.

Other polypeptide variants are those in which one or more amino acid residues include a substituent group. Still other polypeptide variants are those in which the polypeptide is associated with another compound, such as a compound to increase the half-life of the polypeptide (e.g., polyethylene glycol).

Additional polypeptide variants are those in which additional amino acids are fused to the polypeptide, such as a leader sequence, a secretory sequence, a proprotein sequence, or a sequence which facilitates purification, enrichment, or stabilization of the polypeptide.

In some instances, the polypeptide variants described herein retain the same biological function as a polypeptide from which they are derived (e.g., retain fatty aldehyde biosynthetic activity, such as carboxylic acid or fatty acid reductase activity) and have amino acid sequences substantially identical thereto.

In other instances, the polypeptide variants have at least about 50%, at least about 55%, at least about 60%, at least about 65%, at least about 70%, at least about 75%, at least about 80%, at least about 85%, at least about 90%, at least about 95%, or more than about 95% homology to an amino acid sequence from which they are derived. In another embodiment, the polypeptide variants include a fragment comprising at least about 5, 10, 15, 20, 25, 30, 35, 40, 50, 75, 100, or 150 consecutive amino acids thereof.

The polypeptide variants or fragments thereof can be obtained by isolating nucleic acids encoding them using techniques described herein or by expressing synthetic nucleic acids encoding them. Alternatively, polypeptide variants or fragments thereof can be obtained through biochemical enrichment or purification procedures. The sequence of polypeptide variants or fragments can be determined by proteolytic digestion, gel electrophoresis, and/or microsequencing. The sequence of the polypeptide variants or fragments can then be compared to the amino acid sequence from which it is derived using any of the programs described herein.

The polypeptide variants and fragments thereof can be assayed for enzymatic activity, such as fatty aldehyde-producing activity, using routine methods. For example, the polypeptide variants or fragments can be contacted with a substrate (e.g., a fatty acid, a fatty acid derivative substrate, or other substrate described herein) under conditions that allow the polypeptide variants or fragments to function. A decrease in the level of the substrate or an increase in the level of a fatty aldehyde can be measured to determine fatty aldehyde-producing activity.

Production of Fatty Alcohols from Acyl-CoA

Acyl-CoA is reduced to a fatty aldehyde by NADH-dependent acyl-CoA reductase (e.g., Acr1). The fatty aldehyde is then reduced to a fatty alcohol by NADPH-dependent alcohol dehydrogenase (e.g., YqhD). Alternatively, fatty alcohol forming acyl-CoA reductase (FAR) catalyzes the reduction of an acyl-CoA into a fatty alcohol and CoASH. FAR uses NADH or NADPH as a cofactor in this four-electron reduction. Although the alcohol-generating FAR reactions proceed through an aldehyde intermediate, a free aldehyde is not released. Thus, the alcohol-forming FAQs are distinct from those enzymes that carry out two-electron reductions of acyl-CoA and yield free fatty aldehyde as a product. (See Cheng and Russell, J. Biol. Chem., 279(3607789-37797, 2004; Metz et al., Plant Physiol, 122:635-644, 2000).

### Modifications to Increase Conversion of Acyl-CoA to Fatty Alcohol

Production hosts can be engineered using known polypeptides to produce fatty alcohols from acyl-CoA. One method of making fatty alcohols involves increasing the expression of, or expressing more active forms of, fatty alcohol forming acyl-CoA reductases (encode by a gene such as acr1 from FAR, EC 1.2.1.50/1.1.1) or acyl-CoA reductases (EC 1.2.1.50) and alcohol dehydrogenase (EC 1.1.1.1). Exemplary GenBank Accession Numbers are provided in Table A.

For fatty alcohol production, the production host is modified so that it includes a first exogenous DNA sequence encoding a protein capable of converting a fatty acid to a fatty aldehyde and a second exogenous DNA sequence encoding a protein capable of converting a fatty aldehyde to a fatty alcohol. In some examples, the first exogenous DNA sequence encodes a fatty acid reductase. In one embodiment, the second exogenous DNA sequence encodes mammalian microsomal aldehyde reductase or long-chain alcohol dehydrogenase. In a further example, the first and second exogenous DNA sequences are from Arthrobacter AK 19, Rhodotorula glutinins, Acinetobacter sp. strain M-1, or Candida lipolytica. In one embodiment, the first and second heterologous DNA sequences are from a multienzyme complex from Acinetobacter sp. strain M-1 or Candida lipolytica.

Additional sources of heterologous DNA sequences encoding fatty acid to long chain alcohol converting proteins that can be used in fatty alcohol production include, but are not limited to, Mortierella alpina (ATCC 32222), Cryptococcus curvatus, (also referred to as Apiotricum curvatum), Alcanivorax jadensis (T9T=DSM 12718=ATCC 700854), Acinetobacter sp. HO1 N (ATCC 14987) and Rhodococcus opacus (PD630 DSMZ 44193).

### Anti-Fatty Aldehyde Biosynthetic Polypeptide Antibodies

The fatty aldehyde biosynthetic polypeptides described herein can also be used to produce antibodies directed against fatty aldehyde biosynthetic polypeptides. Such antibodies can be used, for example, to detect the expression of a fatty aldehyde, biosynthetic polypeptide using methods known in the art. The antibody can be, for example, a polyclonal antibody; a monoclonal antibody or antigen binding fragment thereof; a modified antibody such as a chimeric antibody, reshaped antibody, humanized antibody, or fragment thereof (e.g., Fab′, Fab, F(ab′)₂); or a biosynthetic antibody, for example, a single chain antibody, single domain antibody (DAB), Fv, single chain Fv (scFv), or the like.

Methods of making and using polyclonal and monoclonal antibodies are described, for example, in Harlow et al., Using Antibodies: A Laboratory Manual: Portable Protocol I. Cold Spring Harbor Laboratory (Dec. 1, 1998). Methods for making modified antibodies and antibody fragments (e.g., chimeric antibodies, reshaped antibodies, humanized antibodies, or fragments thereof, e.g., Fab′, Fab, F(ab′)₂ fragments) or biosynthetic antibodies (e.g., single chain antibodies, single domain antibodies (DABs). FV, single chain Fv (scFv), and the like) are known in the art and can be found, for example, in Zola, Monoclonal Antibodies: Preparation and Use of Monoclonal Antibodies and Engineered Antibody Derivatives, Springer Verlag (Dec. 15, 2000; 1st edition).

### Production of Fatty Alcohols

A fatty aldehyde described herein can be converted into a fatty alcohol by an alcohol dehydrogenase. In some examples, a gene encoding a fatty aldehyde biosynthetic polypeptide described herein can be expressed in a host cell that expresses an endogenous alcohol dehydrogenase capable of converting a fatty aldehyde produced by the fatty aldehyde biosynthetic polypeptide into a corresponding fatty alcohol. Such alcohol dehydrogenases include, but are not limited to, AlrA of Acenitobacter sp. M-1 or AlrA homologs, and endogenous E. coli alcohol dehydrogenases such as DkgA (NP_{—}417485), DkgB (NP_{—}414743). YjgB, (AAC77226), YdjL (AAC74846), YdjJ (NP_{—}416288), AdhP (NP_{—}415995). YhdH (NP_{—}417719), YahK (NP_{—}414859), and YphC (AAC75598). In other instances, a gene encoding an alcohol dehydrogenase can be co-expressed in a host cell with a gene encoding a fatty aldehyde biosynthetic polypeptide described herein.

### Genetic Engineering of Host Cells to Produce Fatty Alcohols

Various host cells can be used to produce fatty alcohols, as described herein. A host cell can be any prokaryotic or eukaryotic cell. For example, a gene encoding a polypeptide described herein (e.g., a fatty aldehyde biosynthetic polypeptide and/or an alcohol dehydrogenase) can be expressed in bacterial cells (such as E. coli), insect cells, yeast, or mammalian cells (such as Chinese hamster ovary cells (CHO) cells, COS cells, VERO cells, BHK cells, HeLa cells, Cv1 cells. MDCK cells, 293 cells, 3T3 cells, or PC12 cells), Other exemplary host cells include cells from the members of the genus Escherichia, Bacillus, Lactobacillus, Rhodococcus, Pseudomonos, Aspergillus, Trichoderma, Neurospora, Fusariutn, Humicola, Rhizomucor, Kluyveromyces, Pichia, Mucor, Myceliophtora, Penicillium, Phanerochaete, Pleurotus, Trametes, Chrysosporiurn, Saccharomyces, Schizosaccharomyces, Yarrowia, or Streptomyces. Yet other exemplary host cells can be a Bacillus lentus cell, a Bacillus brevis cell, a Bacillus stearothermophilus cell, a Bacillus licheniformis cell, a Bacillus alkalophilus cell, a Bacillus coagulans cell, a Bacillus circulans cell, a Bacillus pumilis cell, a Bacillus thuringiensis cell, a Bacillus clausii cell, a Bacillus megaterium cell, a Bacillus subtilis cell, a Bacillus amyloliquefaciens cell, a Trichoderma koningii cell, a Trichoderma viride cell, a Trichoderma reesei cell, a Trichoderma longibrachiatum cell, an Aspergillus awamori cell, an Aspergillus fumigates cell, an Aspergillus foetidus cell, an Aspergillus nidulans cell, an Aspergillus niger cell, an Aspergillus oryzae cell, a Humicola insolens cell, a Humicola lanuginose cell, a Rhizomucor miehei cell, a Mucor michei cell, a Streptomyces lividans cell, a Streptomyces murinus cell, or an Actinomycetes cell. Other host cells are cyanobacterial host cells.

In a preferred embodiment, the host cell is an E. coli cell, a Saccharomyces cerevisiae cell, or a Bacillus subtilis cell. In a more preferred embodiment, the host cell is from E. coli strain B, C, K, or W. Other suitable host cells are known to those skilled in the art.

Various methods well known in the art can be used to genetically engineer host cells to produce fatty alcohols. The methods can include the use of vectors, preferably expression vectors, containing a nucleic acid encoding a fatty aldehyde biosynthetic polypeptide and/or an alcohol dehydrogenase described herein, polypeptide variant, or a fragment thereof. Those skilled in the art will appreciate a variety of viral vectors (for example, retroviral vectors, lentiviral vectors, adenoviral vectors, and adeno-associated viral vectors) and nonviral vectors can be used in the methods described herein.

The recombinant expression vectors described herein include a nucleic acid described herein in a form suitable for expression of the nucleic acid in a host cell. The recombinant expression vectors can include one or more control sequences, selected on the basis of the host cell to be used for expression. The control sequence is operably linked to the nucleic acid sequence to be expressed. Such control sequences are described, for example, in Goeddel, Gene Expression Technology: Methods in Enzymology 185, Academic Press, San Diego, Calif. (1990). Control sequences include those that direct constitutive expression of a nucleotide sequence in many types of host cells and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors described herein can be introduced into host cells to produce polypeptides, including fusion polypeptides, encoded by the nucleic acids as described herein.

Recombinant expression vectors can be designed for expression of a gene encoding a fatty aldehyde biosynthetic polypeptide (or variant) and/or a gene encoding an alcohol dehydrogenase in prokaryotic or eukaryotic cells (e.g., bacterial cells, such as E. coli, insect cells (e.g., using baculovirus expression vectors), yeast cells, or mammalian cells). Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example, by using T7 promoter regulatory sequences and T7 polymerase.

Expression of genes encoding polypeptides in prokaryotes, for example, E. coli, is most often carried out with vectors containing constitutive or inducible promoters directing the expression of either fusion or non-fusion polypeptides. Fusion vectors add a number of amino acids to a polypeptide encoded therein, usually to the amino terminus of the recombinant polypeptide. Such fusion vectors typically serve three purposes: (1) to increase expression of the recombinant polypeptide; (2) to increase the solubility of the recombinant polypeptide; and (3) to aid in the purification of the recombinant polypeptide by acting as a ligand in affinity purification. Often, in fusion expression vectors, a proteolytic cleavage site is introduced at the junction of the fusion moiety and the recombinant polypeptide. This enables separation of the recombinant polypeptide from the fusion moiety after purification of the fusion polypeptide. Examples of such enzymes, and their cognate recognition sequences, include Factor Xa, thrombin, and enterokinase. Exemplary fusion expression vectors include pGEX (Pharmacia Biotech Inc.; Smith et al., Gene, 67: 31-40 (1988)), pMAL (New England Biolabs, Beverly, Mass.), and pRITS (Pharmacia, Piscataway, N.J.), which fuse glutathione S-transferase (GST), maltose E binding protein, or protein A, respectively, to the target recombinant polypeptide.

Examples of inducible, non-fusion E. coli expression vectors include pTrc (Amann et al. Gene, 69: 301-315 (1988)) and pET 11d (Studier et al., Gene Expression Technology: Methods in Enzymology 185, Academic Press, San Diego, Calif. (1990), pp. 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident λ prophage harboring a T7 gn1 gene under the transcriptional control of the lacUV 5 promoter.

One strategy to maximize recombinant polypeptide expression is to express the polypeptide in a host cell with an impaired capacity to proteolytically cleave the recombinant polypeptide (see Gottesman, Gene Expression Technology: Methods in Enzymology 185, Academic Press, San Diego, Calif. (1990), pp. 119-128). Another strategy is to alter the nucleic acid sequence to be inserted into an expression vector so that the individual codons for each amino acid are those preferentially utilized in the host cell (Wada et al., Nucleic Acids Res., 20: 2111-2118 (1992)). Such alteration of nucleic acid sequences can be carried out by standard DNA synthesis techniques.

In another embodiment, the host cell is a yeast cell. In this embodiment, the expression vector is a yeast expression vector. Examples of vectors for expression in yeast S. cerevisiae include pYepSec1 (Baldari et al., EMBO J., 6: 229-234 (1987)), pMFa (Kurjan et al., Cell, 30: 933-943 (1982)), pJRY88 (Schultz et al., Gene, 54: 113-123 (1987)), pYES2 (Invitrogen Corporation, San Diego, Calif.), and picZ (Invitrogen Corp, San Diego, Calif.).

Alternatively, a polypeptide described herein can be expressed in insect cells using baculovirus expression vectors. Baculovirus vectors available for expression of proteins in cultured insect cells (e.g., Sf9 cells) include, for example, the pAc series (Smith et al., Mol. Cell Biol., 3: 2156-2165 (1983)) and the pVL series (Lucklow et al., Virology. 170: 31-39 (1989)).

In yet another embodiment, the nucleic acids described herein can be expressed in mammalian cells using a mammalian expression vector. Examples of mammalian expression vectors include pCDM8 (Seed, Nature, 329: 840 (1987)) and pMT2PC (Kaufman et al. EMBO J., 6: 187-195 (1987)). When used in mammalian cells, the expression vector's control functions can be provided by viral regulatory elements. For example, commonly used promoters are derived from polyoma, Adenovirus 2, cytomegalovirus, and Simian Virus 40. Other suitable expression systems for both prokaryotic and eukaryotic cells are described in chapters 16 and 17 of Sambrook et al., eds., Molecular Cloning: A Laboratory Manual. 2nd, ed., Cold Spring Harbor Laboratory, Cold Spring Harbor Laboratory Press, Cold Spring Harbor, N.Y., 1989.

Vectors can be introduced into prokaryotic or eukaryotic cells via conventional transformation or transfection techniques. As used herein, the terms “transformation” and “transfection” refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in, for example, Sambrook et al. (supra).

For stable transformation of bacterial cells, it is known that, depending upon the expression vector and transformation technique used, only a small fraction of cells will take-up and replicate the expression vector. In order to identify and select these transformants, a gene that encodes a selectable marker (e.g., resistance to antibiotics) can be introduced into the host cells along with the gene of interest. Selectable markers include those that confer resistance to drugs, such as ampicillin, kanamycin, chloramphenicol, or tetracycline. Nucleic acids encoding a selectable marker can be introduced into a host cell on the same vector as that encoding a polypeptide described herein or can be introduced on a separate vector. Cells stably transfected with the introduced nucleic acid can be identified by drug selection (e.g., cells that have incorporated the selectable marker gene will survive, while the other cells die).

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) can be introduced into the host cells along with the gene of interest. Preferred selectable markers include those which confer resistance to drugs, such as G418, hygromycin, and methotrexate. Nucleic acids encoding a selectable marker can be introduced into a host cell on the same vector as that encoding a polypeptide described herein or can be introduced on a separate vector. Cells stably transfected with the introduced nucleic acid can be identified by drug selection (e.g., cells that have incorporated the selectable marker gene will survive, while the other cells die).

### Transport Proteins

Transport proteins can export polypeptides and organic compounds (e.g., fatty alcohols) out of a host cell. Many transport and efflux proteins serve to excrete a wide variety of compounds and can be naturally modified to be selective for particular types of hydrocarbons.

Non-limiting examples of suitable transport proteins are ATP-Binding Cassette (ABC) transport proteins, efflux proteins, and fatty acid transporter proteins (FATP). Additional non-limiting examples of suitable transport proteins include the ABC transport proteins from organisms such as Caenorhabdais elegans, Arabidopsis thalania, Alkaligenes eutrophus, and Rhodococcus erythropolis. Exemplary ABC transport proteins that can be used are listed in Table A (e.g., CER5, AtMRP5, AmiS2, and AtPGP1), Host cells can also be chosen for their endogenous ability to secrete organic compounds. The efficiency of organic compound production and secretion into the host cell environment (e.g., culture medium, fermentation broth) can be expressed as a ratio of intracellular product to extracellular product. In some examples, the ratio can be about 5:1, 4:1, 3:1, 2:1, 1:1, 1:2, 1:3, 1:4, or 1:5.

### Fermentation

The production and isolation of fatty alcohols can be enhanced by employing beneficial fermentation techniques. One method for maximizing production while reducing costs is increasing the percentage of the carbon source that is converted to hydrocarbon products.

During normal cellular lifecycles, carbon is used in cellular functions, such as producing lipids, saccharides, proteins, organic acids, and nucleic acids. Reducing the amount of carbon necessary for growth-related activities can increase the efficiency of carbon source conversion to product. This can be achieved by, for example, first growing host cells to a desired density (for example, a density achieved at the peak of the log phase of growth). At such a point, replication checkpoint genes can be harnessed to stop the growth of cells. Specifically, quorum sensing mechanisms (reviewed in Camilli et al., Science, 311:1113 (2006); Venturi FEMS Microbio. Rev., 30: 274-291 (2006); and Reading et al., FEMS Microbiol. Lett., 254: 1-11 (2006)) can be used to activate checkpoint genes, such as p53, p21, or other checkpoint genes.

Genes that can be activated to stop cell replication and growth in E. coli include umuDC genes. The overexpression of umuDC genes stops the progression from stationary phase to exponential growth (Murli et al., J. Bact., 182: 1127 (2000)). UmuC is a DNA polymerase that can carry out translesion synthesis over non-coding lesions—the mechanistic basis of most UV and chemical mutagenesis. The umuDC gene products are involved in the process of translesion synthesis and also serve as a DNA sequence damage checkpoint. The umuDC gene products include UmuC, UmuD, umuD′, UmuD′₂C, UmuD′₂, and UmuD₂. Simultaneously, product-producing genes can be activated, thus minimizing the need for replication and maintenance pathways to be used while a fatty aldehyde is being made. Host cells can also be engineered to express umuC and umuD from E. coli in pBAD24 under the prpBCDE promoter system through de novo synthesis of this gene with the appropriate end-product production genes.

The percentage of input carbons converted to fatty alcohols can be a cost driver. The more efficient the process is (i.e., higher the percentage of input carbons converted to fatty alcohols), the less expensive the process will be. For oxygen-containing carbon sources (e.g., glucose and other carbohydrate based sources), the oxygen must be released in the form of carbon dioxide. For every 2 oxygen atoms released, a carbon atom is also released leading to a maximal theoretical metabolic efficiency of approximately 34% (w/w) (for fatty acid derived products). This figure, however, changes for other organic compounds and carbon sources. Typical efficiencies in the literature are approximately less than 5%. Host cells engineered to produce fatty alcohols can have greater than about 1, 3, 5, 10, 15, 20, 25, and 30% efficiency. In one example, host cells can exhibit an efficiency of about 10% to about 25%. In other examples, such host cells can exhibit an efficiency of about 25% to about 30%. In other examples, host cells can exhibit greater than 30% efficiency.

The host cell can be additionally engineered to express recombinant cellulosomes, such as those described in PCT Publication No. WO 2008/100251. These cellulosomes can allow the host cell to use cellulosic material as a carbon source. For example, the host cell can be additionally engineered to express invertases (EC 3.2.26) so that sucrose can be used as a carbon source. Similarly, the host cell can be engineered using the teachings described in U.S. Pat. Nos. 5,000,000; 5,028,539; 5,424,202; 5,482,846; and 5,602,030, so that the host cell can assimilate carbon efficiently and use cellulosic materials as carbon sources.

In one example, the fermentation chamber can enclose a fermentation that is undergoing a continuous reduction. In this instance, a stable reductive environment can be created. The electron balance can be maintained by the release of carbon dioxide (in gaseous form). Efforts to augment the NAD/H and NADP/H balance can also facilitate in stabilizing the electron balance. The availability of intracellular NADPH can also be enhanced by engineering the host cell to express an NADH:NADPH transhydrogenase. The expression of one or more NADH:NADPH transhydrogenases converts the NADH produced in glycolysis to NADPH, which can enhance the production of fatty alcohols.

For small scale production, the engineered host cells can be (a) grown in batches of, for example, about 100 mL, 500 mL, 1 L, 2 L, 5 L, or 10 L, (b) fermented, and (c) induced to express desired fatty aldehyde biosynthetic genes and/or an alcohol dehydrogenase genes based on the specific genes encoded in the appropriate plasmids. For large scale production, the engineered host cells can be (a) grown in batches of about 10 L, 100 L, 1000 L, 10,000 L, 100,000 L, 1,000,000 L, or larger, (b) fermented, and (c) induced to express desired fatty aldehyde biosynthetic genes and/or alcohol dehydrogenase genes based on the specific genes encoded in the appropriate plasmids or incorporated into the host cell's genome.

For example, a suitable production host, such as E. coli cells, harboring plasmids containing the desired genes or having the genes integrated in its chromosome can be incubated in a suitable reactor, for example a 1 L reactor, for 20 hours at 37° C. in M9 medium supplemented with 2% glucose, carbenicillin, and chloramphenicol. When the OD₆₀₀ of the culture reaches 0.9, the production host can be induced with IPTG alcohol After incubation, the spent media can be extracted and the organic phase can be examined for the presence of fatty alcohols using GC-MS.

In some instances, after the first hour of induction, aliquots of no more than about 10% of the total cell volume can be removed each hour and allowed to sit without agitation to allow the fatty alcohols to rise to the surface and undergo a spontaneous phase separation or precipitation. The fatty alcohol component can then be collected, and the aqueous phase returned to the reaction chamber. The reaction chamber can be operated continuously. When the OD₆₀₀ drops below 0.6, the cells can be replaced with a new batch grown from a seed culture.

### Producing Fatty Alcohols Using Cell-Free Methods

In some methods described herein, a fatty alcohol can be produced using a purified polypeptide (e.g., an alcohol dehydrogenases) described herein and a substrate (e.g., fatty aldehyde), produced, for example, by a method described herein. For example, a host cell can be engineered to express a biosynthetic polypeptide (e.g., an alcohol dehydrogenase) or variant as described herein. The host cell can be cultured under conditions suitable to allow expression of the polypeptide. Cell free extracts can then be generated using known methods. For example, the host cells can be lysed using detergents or by sonication. The expressed polypeptides can be purified using known methods. After obtaining the cell free extracts, substrates described herein can be added to the cell free extracts and maintained under conditions to allow conversion of the substrates (e.g., fatty aldehydes) to fatty alcohols. The fatty alcohols can then be separated and purified using known techniques.

In some instances, a fatty aldehyde described herein can be converted into a fatty alcohol by contacting the fatty aldehyde with an alcohol dehydrogenase. Such alcohol dehydrogenases include, but are not limited to, AlrA of Acenitobacter sp. M-1 or AlrA homologs, and endogenous E. coli alcohol dehydrogenases such as DkgA (NP_{—}417485), DkgB (NP_{—}414743), YjgB, (AAC77226), YdjL (AAC74846), YdjJ (NP_{—}416288), AdhP (NP_{—}415995), YhdH (NP_{—}417719), YahK (NP_{—}414859), and YphC (AAC75598).

### Post-Production Processing

The fatty alcohols produced during fermentation can be separated from the fermentation media. Any known technique for separating fatty alcohols from aqueous media can be used. One exemplary separation process is a two phase (bi-phasic) separation process. This process involves fermenting the genetically engineered host cells under conditions sufficient to produce a fatty alcohol, allowing the fatty alcohol to collect in an organic phase, and separating the organic phase from the aqueous fermentation broth. This method can be practiced in both a batch and continuous fermentation processes.

Bi-phasic separation uses the relative immiscibility of fatty alcohols to facilitate separation. Immiscible refers to the relative inability of a compound to dissolve in water and is defined by the compound's partition coefficient. One of ordinary skill in the art will appreciate that by choosing a fermentation broth and organic phase, such that the fatty alcohol being produced has a high logP value, the fatty alcohol can separate into the organic phase, even at very low concentrations, in the fermentation vessel.

The fatty alcohols produced by the methods described herein can be relatively immiscible in the fermentation broth, as well as in the cytoplasm. Therefore, the fatty alcohol can collect in an organic phase either intracellularly or extracellularly. The collection of the products in the organic phase can lessen the impact of the fatty alcohol on cellular function and can allow the host cell to produce more product.

The methods described herein can result in the production of homogeneous compounds wherein at least about 60%, 70%, 80%, 90%, or 95% of the fatty alcohols produced will have carbon chain lengths that vary by less than about 6 carbons, less than about 4 carbons, or less than about 2 carbons. These compounds can also be produced with a relatively uniform degree of saturation. These compounds can be used directly as fuels, fuel additives, starting materials for production of other chemical compounds (e.g., polymers, surfactants, plastics, textiles, solvents, adhesives, etc.), or personal care additives. These compounds can also be used as feedstock for subsequent reactions, for example, hydrogenation, catalytic cracking (e.g., via hydrogenation, pyrolysis, or both), and dehydration to make other products.

In some embodiments, the fatty alcohols produced using methods described herein can contain between about 50% and about 90% carbon, or between about 5% and about 25% hydrogen. In other embodiments, the fatty alcohols produced using methods described herein can contain between about 65% and about 85% carbon, or between about 10% and about 15% hydrogen.

Bioproducts (e.g., fatty alcohols) comprising biologically produced organic compounds, particularly fatty alcohols biologically produced using the fatty acid biosynthetic pathway, have not been produced from renewable sources and, as such, are new compositions of matter. These new bioproducts can be distinguished from organic compounds derived from petrochemical carbon on the basis of dual carbon-isotopic fingerprinting or ¹⁴C dating. Additionally, the specific source of biosourced carbon (e.g., glucose vs. glycerol) can be determined by dual carbon-isotopic fingerprinting (see, e.g., U.S. Pat. No. 7,169,588).

The ability to distinguish bioproducts from petroleum based organic compounds is beneficial in tracking these materials in commerce. For example, organic compounds or chemicals comprising both biologically based and petroleum based carbon isotope profiles may be distinguished from organic compounds and chemicals made only of petroleum based materials. Hence, the instant materials may be followed in commerce on the basis of their unique carbon isotope profile.

Bioproducts can be distinguished from petroleum based organic compounds by comparing the stable carbon isotope ratio (¹³C/¹²C) in each fuel. The ¹³C/¹²C ratio in a given bioproduct is a consequence of the ¹³C/¹²C ratio in atmospheric carbon dioxide at the time the carbon dioxide is fixed. It also reflects the precise metabolic pathway. Regional variations also occur. Petroleum, C₃ plants (the broadleaf), C₄ plants (the grasses), and marine carbonates all show significant differences in ¹³C/¹²C and the corresponding δ¹³C values. Furthermore, lipid matter of C₃ and C₄ plants analyze differently than materials derived from the carbohydrate components of the same plants as a consequence of the metabolic pathway.

Within the precision of measurement, ¹³C shows large variations due to isotopic fractionation effects, the most significant of which for bioproducts is the photosynthetic mechanism. The major cause of differences in the carbon isotope ratio in plants is closely associated with differences in the pathway of photosynthetic carbon metabolism in the plants, particularly the reaction occurring during the primary carboxylation (i.e., the initial fixation of atmospheric CO₂). Two large classes of vegetation are those that incorporate the “C₃” (or Calvin-Benson) photosynthetic cycle and those that incorporate the “C₄” (or Hatch-Slack) photosynthetic cycle.

In C₃ plants, the primary CO₂ fixation or carboxylation reaction involves the enzyme ribulose-1,5-diphosphate carboxylase, and the first stable product is a 3-carbon compound. C₃ plants, such as hardwoods and conifers, are dominant in the temperate climate zones.

In C₄ plants, an additional carboxylation reaction involving another enzyme, phosphoenol-pyruvate carboxylase, is the primary carboxylation reaction. The first stable carbon compound is a 4-carbon acid that is subsequently decarboxylated. The CO₂ thus released is refixed by the C₃ cycle. Examples of C₄ plants are tropical grasses, corn, and sugar cane.

Both C₄ and C₃ plants exhibit a range of ¹³C/¹²C isotopic ratios, but typical values are about −7 to about −13 per mil for C₄ plants and about −19 to about −27 per mil for C₃ plants (see. e.g., Stuiver et al., Radiocarbon, 19: 355 (1977)). Coal and petroleum fall generally in this latter range. The ¹³C measurement scale was originally defined by a zero set by Pee Dee Belemnite (PDB) limestone, where values are given in parts per thousand deviations from this material. The “δ¹³C” values are expressed in parts per thousand (per mil), abbreviated, ‰, and are calculated as follows:

δ¹³C(‰)=[(¹³C/¹²C)ₛₐₘₚₗₑ−(¹³C/¹²C)_{standard}]/(¹³C/¹²C)_{standard}×1000

Since the PDB reference material (RM) has been exhausted, a series of alternative RMs have been developed in cooperation with the IAEA, USGS, NIST, and other selected international isotope laboratories. Notations for the per mil deviations from PDB is δ¹³C. Measurements are made on CO₂ by high precision stable ratio mass spectrometry (IRMS) on molecular ions of masses 44, 45, and 46.

The compositions described herein include bioproducts produced by any of the methods described herein. Specifically, the bioproduct can have a δ¹³C of about −28 or greater, about −27 or greater, −20 or greater, −18 or greater, −15.4 or greater, −15 or greater, −13 or greater, −10 or greater, or −8 or greater. For example, the bioproduct can have a δ¹³C of about −30 to about −15, about −27 to about −19, about −25 to about −21, about −15 to about −5, about −15.4 to about −10.9, about −13.92 to about −13.84, about −13 to about −7, or about −13 to about −10. In other instances, the bioproduct can have a δ¹³C of about −10, −11, −12, or −12.3.

Bioproducts can also be distinguished from petroleum based organic compounds by comparing the amount of ¹⁴C in each compound. Because ¹⁴C has a nuclear half life of 5730 years, petroleum based fuels containing “older” carbon can be distinguished from bioproducts which contain “newer” carbon (see, e.g., Currie, “Source Apportionment of Atmospheric Particles,” Characterization of Environmental Particles, J. Buffle and H. P. van Leeuwen, Eds., 1 of Vol. I of the IUPAC Environmental Analytical Chemistry Series (Lewis Publishers, Inc.) (1992), pp. 3-74).

The basic assumption in radiocarbon dating is that the constancy of ¹⁴C concentration in the atmosphere leads to the constancy of ¹⁴C in living organisms. However, because of atmospheric nuclear testing since 1950 and the burning of fossil fuel since 1850, ¹⁴C has acquired a second, geochemical time characteristic. Its concentration in atmospheric CO₂, and hence in the living biosphere, approximately doubled at the peak of nuclear testing, in the mid-1960s. It has since been gradually returning to the steady-state cosmogenic (atmospheric) baseline isotope rate (¹⁴C/¹²C) of about 1.2×10⁻¹², with an approximate relaxation “half-life” of 7-10 years. (This latter half-life must not be taken literally; rather, one must use the detailed atmospheric nuclear input/decay function to trace the variation of atmospheric and biospheric ¹⁴C since the onset of the nuclear age.)

It is this latter biospheric ¹⁴C time characteristic that holds out the promise of annual dating of recent biospheric carbon. ¹⁴C can be measured by accelerator mass spectrometry (AMS), with results given in units of “fraction of modern carbon” (f_{M}). As used herein, “fraction of modern carbon” or “f_{M}” has the same meaning as defined by National Institute of Standards and Technology (NIST) Standard Reference Materials (SRMs) 4990B and 4990C, known as oxalic acids standards HOxI and HOxII, respectively. The fundamental definition relates to 0.95 times the ¹⁴C/¹²C isotope ratio HOxI (referenced to AD 1950). This is roughly equivalent to decay-corrected pre-Industrial Revolution wood. For the current living biosphere (plant material), f_{M} is approximately 1.1.

The invention provides a bioproduct which can have an f_{M} ¹⁴C of at least about 1. For example, the bioproduct can have an f_{M} ¹⁴C of at least about 1.01, of at least about 1.5, an f_{M} ¹⁴C of about 1 to about 1.5, an f_{M} ¹⁴C of about 1.04 to about 1.18, or an f_{M} ¹⁴C of about 1.111 to about 1.124.

Another measurement of ¹⁴C is known as the percent of modern carbon, pMC. For an archaeologist or geologist using ¹⁴C dates, AD 1950 equals “zero years old”. This also represents 100 pMC. “Bomb carbon” in the atmosphere reached almost twice the normal level in 1963 at the peak of thermonuclear weapons testing. Its distribution within the atmosphere has been approximated since its appearance, showing values that are greater than 100 pMC for plants and animals living since AD 1950. It has gradually decreased over time with today's value being near 107.5 pMC. This means that a fresh biomass material, such as corn, would give a ¹⁴C signature near 107.5 pMC. Petroleum based compounds will have a pMC value of zero. Combining fossil carbon with present day carbon will result in a dilution of the present day pMC content. By presuming 107.5 pMC represents the ¹⁴C content of present day biomass materials and 0 pMC represents the ¹⁴C content of petroleum based products, the measured pMC value for that material will reflect the proportions of the two component types. For example, a material derived 100% from present day soybeans would give a radiocarbon signature near 107.5 pMC. If that material was diluted 50% with petroleum based products, it would give a radiocarbon signature of approximately 54 pMC.

A biologically based carbon content is derived by assigning “100%” equal to 107.5 pMC and “0%” equal to 0 pMC. For example, a sample measuring 99 pMC will give an equivalent biologically based carbon content of 93%. This value is referred to as the mean biologically based carbon result and assumes all the components within the analyzed material originated either from present day biological material or petroleum based material.

A bioproduct described herein can have a pMC of at least about 50, 60, 70, 75, 80, 85, 90, 95, 96, 97, 98, 99, or 100. In other instances, a bioproduct described herein can have a pMC of between about 50 and about 100; between about 60 and about 100; between about 70 and about 100; between about 80 and about 100: between about 85 and about 100; between about 87 and about 98; or between about 90 and about 95. In yet other instances, a bioproduct described herein can have a pMC of about 90, 91, 92, 93, 94, or 94.2.

### Fatty Alcohol Derivatives

The fatty alcohol derivative of the fatty alcohol is produced by converting the isolated fatty alcohol into a fatty alcohol derivative thereof. The fatty alcohol derivative can be any suitable fatty alcohol derivative and, for example, comprises a fatty ether sulfate, a fatty phosphate ester, an alkylbenzyldimethylammonium chloride, a fatty amine oxide, a fatty alcohol sulfate, an alkyl polyglucoside, an alkyl glyceryl ether sulfonate, or an ethoxylated fatty alcohol. Typically, the fatty alcohol derivative comprises an alkyl group that is about 6 to about 26 carbons in length. In one embodiment the alkyl group contains an even number of carbons. In one such embodiment, the fatty alcohol comprises an alkyl group that is about 8, 10, 12, 14, 16, or 18 carbons in length. In another embodiment, the alkyl group contains an odd number of carbons. In this regard, the number of carbons recited for the alkyl group refers to the hydrocarbon group that is derived from the fatty alcohol, and not to any carbon atoms added in the preparation of the fatty alcohol derivative, such as polyethoxy groups and the like.

As used herein, the term “fatty ether sulfate” is the same as “alkyl ether sulfate” wherein the alkyl residue is a fatty residue, and denotes a compound of the structure: RO(CH₂CH₂O)ₙ—SO₃H wherein R is a C₆-C₂₆ alkyl group as defined herein and n is an integer of 1 to about 50. Fatty ether sulfates can also refer to the salt of the above structure, which is denoted by RO(CH₂CH₂O)ₙSO₃X, where n and R are as defined herein and X is a cation. An exemplary fatty ether sulfate salt is a sodium salt, for example, RO(CH₂CH₂O)ₙSO₃Na.

As used herein, the term “fatty alcohol sulfate” denotes a compound of the structure: ROSO₃H wherein R is a C₆-C₂₆ alkyl group as defined herein. Fatty alcohol sulfates can also refer to the salt of the above structure, which is denoted by ROSO₃X where R is as defined above and X is a cation. An exemplary fatty alcohol sulfate salt is a sodium salt, for example, ROSO₃Na.

As used herein, the term “fatty phosphate ester” is the same as “alkyl phosphate ester” wherein the alkyl residue is a fatty residue, and denotes compounds of the structures: ROP(O)(OH)₂ and (RO)₂P(O)(OH) and mixtures thereof. Fatty phosphate ester can also refer to the salts of the above structures and mixtures thereof, which are denoted by ROP(O)(OX)₂ and (RO)₂P(O)(OX) where R is as defined above and X is a cation. An exemplary fatty phosphate ester salt is a sodium salt, for example, ROP(ONa)₂.

As used herein, the term “fatty ether phosphate” is the same as “alkyl ether phosphate” wherein the alkyl residue is a fatty residue, and denotes compounds of the structures: RO(CH₂CH₂O)ₙ—P(O)(OH)₂ and (RO(CH₂CH₂O)ₙ)₂—P(O)(OH) and mixtures thereof wherein R is/are C₆-C₂₆ alkyl group(s) as defined herein and n is an integer of 1 to about 50. Fatty ether phosphates can also refer to the salts of the above structures, which are denoted by RO(CH₂CH₂O)ₙ—P(O)(OX)₂ and (RO(CH₂CH₂O)ₙ)₂—P(O)(OX) and mixtures thereof, where n and R are as defined herein and X is a cation. An exemplary fatty ether phosphate salt is a sodium salt, for example, RO(CH₂CH₂O)ₙP(O)(ONa)₂.

As used herein, fatty quaternary surfactants have the structure RN(CH₃)₃Cl or RN(CH₃)ₘ(CH₂CH₂OH)ₙ wherein m+n equals 3 and wherein R is a C₆-C₂₆ alkyl group as defined herein.

As used herein, alkylbenzyldimethylammonium chlorides have the structure:

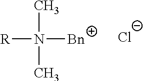

wherein R is a C₆-C₂₆ alkyl group as defined herein.

As used herein, the term “fatty amine oxide” is the same as “alkyl amine oxide” wherein the alkyl residue is a fatty residue as defined herein, and denotes a compound of the structure:

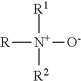

wherein R is a C₆-C₂₆ alkyl group as defined herein and wherein R¹ and R² are C₁-C₂₆ alkyl groups, preferably C₁-C₆ alkyl groups.

As used herein, the term “fatty betaine” is the same as “alkyl betaine” wherein the alkyl residue is a fatty residue as defined herein, and denotes a compound of the structure:

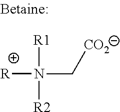

wherein R is a C₆-C₂₆ alkyl group as defined herein and R¹ and R² are C₁-C₂₆ alkyl groups, preferably C₁-C₆ alkyl groups.

As used herein, the term “fatty sultaine” is the same as “alkyl sultaine” wherein the alkyl residue is a fatty residue as defined herein, and denotes a compound of the structure:

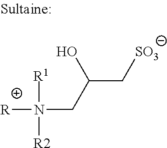

wherein R is a C₆-C₂₆ alkyl group as defined herein and R¹ and R² are C₁-C₂₆ alkyl groups, preferably C₁-C₆ alkyl groups.

Both betaines and sultaines may also contain a counter ion depending on pH of the media in which they are found.

Alkyl polyglucosides have the structure: RO(CₙH₂ₙO)ₜZₓ wherein R is a C₆-C₂₆ alkyl group as defined herein, Z is a glucose residue, n is 2 or 3, t is from 0 to 10, and x is from about 1 to 10, preferably from about 1.5 to 4.

Alkyl glyceryl ether sulfonates have the structure:

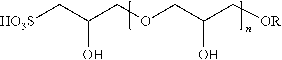

wherein R is a C₆-C₂₆ alkyl group as defined herein, and n is an integer from 0 to 4.

As used herein, the term “fatty alcohol alkoxylate” is the same as “alkoxylated fatty alcohol” and denotes a compound of the structure: RO(CH₂CH₂)ₙOH wherein R is a C₆-C₂₆ alkyl group as defined herein and n is from 1 to about 50.

In addition to the above examples, it is understood that R, wherein R is a C₆-C₂₆ group as defined herein, may be incorporated into a variety of surfactant types including carboxylate, disulfate, sulfonate, disulfonate, glycerol ester sulfonate, amine, monoalkylamine, dialkylamine, glycerol sulfonate, polygluconate, phosphonate, sulfosuccinate, sulfosuccaminate, glucamide, taurinate, sarcosinate, elycinate, isethionate, dialkanol amide, monoalkanol amide, monoalkanol amide sulfate, diglycolamide, diglycolamide sulfate, a glycerol ester, a glycerol ester sulfate, a glycerol ether, a glycerol ether sulfate, a polyglycerol ether, a polyglycerol ether sulfate, sorbitan ester, an alkyl, ammonioalkanesulfonate, amidopropyl betaine, glycerol ester quat, a glycol amine quat, or imidazoline.

The fatty alcohol derivative can be produced by any suitable method, many of which are well known in the art. See, for example, “Handbook on Soaps, Detergents, and Acid Slurry,” 2^{nd} ed., NIIR Board, Asia Pacific Business Press, Inc., Delhi, India.

In one embodiment, the fatty alcohol derivative is an ethoxylated fatty alcohol, which is also known in the art as a fatty alcohol ethoxylate, and has a structure as described herein. Preferably, the ethoxylated fatty alcohol contains from about 1 to about 50 moles of ethylene oxide per mole of fatty alcohol.

The alcohols of the invention can be alkoxylated using standard commercial and laboratory techniques and/or sulfated using any convenient sulfating agent, e.g., chlorosulfonic acid. SO₃/air, or oleum, to yield the final alcohol derived surfactant compositions.

The following two analytical methods for characterizing branching in the alcohol derived surfactant compositions are useful:
1) Separation and Identification of Components in Detergent Alcohols (prior to alkoxylation or after hydrolysis of alcohol sulfate for analytical purposes). The position and length of branching found in the precursor detergent alcohol materials is determined by GC/MS techniques [see: D. J. Harvey, Biomed, Environ. Mass Spectrom (1989). 18(9), 719-23; a J. Harvey, J. M. Tiffany, J. Chromatogr. (1984), 301(1), 173-87; K. A. Karlsson, B. E. Samuelsson. G. O, Steen, Chem. Phys. Lipids (1973), 11(1), 17-38].
2) Identification of Separated Detergent Alcohol Alkoxy Sulfate Components by MS/MS. The position and length of branching is also determinable by Ion Spray-MS/MS or FAB-MS/MS techniques on previously isolated detergent alcohol sulfate components.

The average total carbon atoms of the branched primary alkyl surfactants herein can be calculated from the hydroxyl value of the precursor detergent alcohol mix or from the hydroxyl value of the alcohols recovered by extraction after hydrolysis of the alcohol sulfate mix according to common procedures, such as outlined in “Bailey's Industrial Oil and Fat Products”, Volume 2, Fourth Edition, edited b Daniel Swern, pp. 440-441.

### Surfactant Compositions

Suitable microbially produced alcohol derived surfactant compositions can be formulated in any suitable manner, such as, for example, set forth herein.

### Synthesis Example I

### Synthesis of Microbially Produced Alcohol Sulfate and Mixtures Thereof

A reaction vessel that has agitation and a nitrogen purge to exclude air is used while combining 96 grams (0.493 mol, 1.0 mole equivalents) of microbially produced C12/14 (70/30 ratio) fatty alcohol mixture and 149 grams of diethyl ether. The mixture is chilled to −5° C., then 60.3 g grams (0.518 mol, 1.05 mole equivalents) of chlorosulfonic acid [7790-94-5] is added drop-wise while keeping the temperature of the mixture to below 10° C. Vacuum is applied to remove evolving HCl gas while the mixture is allowed to warm to ˜30° C. Diethyl ether is replaced twice as it is evaporated while continuously mixing for two hours. Then the ether is removed by vacuum prior to the next step.

The mixture from above is added slowly with mixing to a stainless steel beaker containing 343 grams of 9% sodium methoxide in methanol (0.572 mol, 1.16 equivalents) that is chilled in an ice bath. The mixture is stirred for an hour then poured into a stainless steel tray. The solvents are then evaporated and the sample further dried using a vacuum oven.

### Synthesis Example II

### Synthesis of Microbially produced 7-Mole Alcohol Ethoxylate (AE7) and Mixtures Thereof

The following describes the reaction of microbially produced alcohols (C12/14 ratio 70/30) with seven molar equivalents of ethylene oxide.

The following materials are charged to a 600 mL stainless steel stirred pressure vessel with a cooling coil: 200 grams (1.027 mole, 1.0 mole equivalents) of microbially produced alcohols plus enough catalyst to facilitate the reaction of the alcohol with ethylene oxide within a suitable period of time and in a controllable manner. In this example, 1.1 grams of a solution consisting of 50% potassium hydroxide in water is used. However, other kinds and quantities of catalyst can be used based upon the demands of the process.

The reactor is heated while applying a vacuum for removing materials that can result in side products, such as water, that may be introduced with the catalyst, at a temperature that will not allow the loss of the microbially produced alcohols, generally between 40° C. and 90° C., but preferable 70° C., when using a water aspirator as a vacuum source. The removal of water is facilitated by using low speed agitation, generally about 50 RPM, while sparging the mixture with a low level (trickle) stream of inert gas either through a bottom drain valve or through a stainless steel gas dispersion frit or any inert dip-tube or sintered metal fritted material or by sweeping the area above the mixture with inert gas. Samples can be drawn from the reactor and analyzed for water content using an appropriate analytical method such as Karl-Fischer titration. However, a period of about two hours for water removal is a suitable period of time based on past experience.

After completion of the water removal step, ethylene oxide is added to the reactor. Ethylene oxide can be added all at once if the reactor system is properly designed to prevent an uncontrolled rate of reaction. However, the best reaction control is obtained by first heating the reactor under a static vacuum (or optionally with added pressure from an inert gas such as nitrogen) to a temperature that is suitable for the reaction of the alcohol-catalyst mixture with ethylene oxide to occur with minimum side products and color generation, generally between 85° and 150° C., but preferably about 120° C.

Once the reactor has reached the desired temperature, 316.3 grams (7.189 mol, 7.0 equivalents) of ethylene oxide is added at a rate that will be controllable by the cooling system, generally over a period of 30 to 60 minutes.

After the addition of ethylene oxide is completed, stirring and heating is continued until the ethylene oxide has been consumed by the reaction, generally requiring 2 to 4 hours when using this specific reactor system and the given set of reaction conditions. The resulting product can be removed and used as is or neutralized with an appropriate acid such as hydrochloric, sulfuric acid, citric, acetic or similar acids. Filtration of the neutralized surfactant may or may not be necessary to avoid solid inorganic salts from contaminating the end use formulation.

### Synthesis Example III

### Synthesis of Microbially Produced 10-Mole Alcohol Ethoxylate (AE10) and Mixtures Thereof

The following describes the reaction of microbially produced alcohols with ten molar equivalents of ethylene oxide.

The equipment and procedure from SYNTHESIS EXAMPLE II is used but the amount of ethylene oxide used is adjusted to the loading of the microbially produced alcohol.

### Synthesis Example IV

### Synthesis of Microbially produced 3-Mole Alcohol Ethoxylate (AE and Mixtures Thereof

The following describes the reaction of microbially produced alcohols with three molar equivalents of ethylene oxide.

The equipment and procedure from SYNTHESIS EXAMPLE II is used but the amount of ethylene oxide used is adjusted to the loading of the microbially produced alcohol.

### Synthesis Example V

### Synthesis of Microbially Produced Alcohol Ethoxylate Sulfate (AE3S) and Mixtures Thereof

A reaction vessel that has agitation and a nitrogen purge to exclude air is used while combining 62 grams (0.190 mole, 1.0 mole equivalents) of the material obtained in SYNTHESIS EXAMPLE IV and 149 grams of diethyl ether. The mixture is chilled to −5° C., then 23.3 grams (0.200 mol, 1.05 mole equivalents) of chlorosulfonic acid [7790-94-5] is added drop-wise while keeping the temperature of the mixture to below 10° C. Vacuum is applied to remove evolving HCl gas while the mixture is allowed to warm to ˜30° C. Diethyl ether is replaced twice as it is evaporated while continuously mixing for two hours. Then the ether is removed by vacuum prior to the next step.

The mixture from above is added slowly with mixing to a stainless steel beaker containing 132.2 grams (0.220 mol, 1.16 equivalents) of 9% sodium methoxide in methanol that is chilled in an ice bath. The mixture is stirred for an hour then poured into a stainless steel tray. The solvents are then evaporated and the sample further dried using a vacuum oven.

### Cleaning Compositions

The cleaning composition can be, for example, agranular detergent, a bar-form detergent, a liquid laundry detergent, a liquid hand dishwashing composition, a sachet, a two in one multi-compartment pouch containing both solid and liquid compartments, a tablet, or a hard surface cleaner. Multi-compartment compositions can comprise multiple phases sleeted from liquids and solids. The cleaning composition typically contains a carrier, such as water or other solvents.

Detergent formulators are continuously faced with the task of devising products to remove abroad spectrum of soils and stains from fabrics. Chemically and physico-chemically, the varieties of soils and stains range the spectrum from polar soils, such as proteinaceous, clay, and inorganic soils, to non-polar soils, such as soot, carbon-black, byproducts of incomplete hydrocarbon combustion, and organic soils. The removal of greasy stains has been a particularly challenging problem. This challenge has been accentuated by the recent high interest and motivation to reduce the level of surfactants in cleaning detergents for environmental sustainability and cost reasons. The reduction of level of surfactants, especially oil-derived surfactants such as linear alkyl benzene sulfonate, LAS, has typically been found to lead to an erosion of greasy stain removal. Additionally, the global trend of using washing conditions at lower temperature further diminishes grease cleaning capabilities of typical detergents.

As a result of these trends, there is a need for new laundry and cleaning ingredients that provide hydrophobic and hydrophilic soil cleaning and whiteness maintenance. The material should exhibit good greasy soil detaching capability. They should also minimize the amount of suspended and emulsified soil from redepositing on the surfaces of the textiles or hard surfaces. Preferably, the new ingredient would also display a synergy with proteases for removing protease-sensitive stains like grass.

The novel surfactant composition comprising one or more derivatives of the detergent alcohol selected from the sulphate, alkoxylated or the alkoxylated sulphate or mixtures thereof according to the present invention are outstandingly suitable as a soil detachment-promoting additives for laundry detergents and cleaning compositions. They exhibit high dissolving power especially in the case of greasy soil. It is of particular advantage that they display the soil-detaching power even at low washing temperatures.

The novel surfactant compositions according to the present invention can be added to the laundry detergents and cleaning compositions in amounts of generally from 0.05 to 70% by weight, preferably from 0.1 to 40% by weight and more preferably from 0.25 to 10% by weight, based on the particular overall composition.

In addition, the laundry detergents and cleaning compositions generally comprise surfactants and, if appropriate, other polymers as washing substances, builders and further customary ingredients, for example cobuilders, complexing agents, bleaches, standardizers, graying inhibitors, dye transfer inhibitors, enzymes and perfumes.

The novel surfactant compositions of the present invention may be utilized in laundry detergents or cleaning compositions comprising a surfactant system comprising C₁₀-C₁₅ alkyl benzene sulfonates (LAS) and one or more co-surfactants selected from nonionic, cationic, anionic or mixtures thereof. The selection of co-surfactant may be dependent upon the desired benefit. In one embodiment, the co-surfactant is selected as a nonionic surfactant, preferably C₁₂-C₁₈ alkyl ethoxylates. In another embodiment, the co-surfactant is selected as an anionic surfactant, preferably C₁₀-C₁₈, alkyl alkoxy sulfates (AEₓS) wherein x is from 1-30. In another embodiment the co-surfactant is selected as a cationic surfactant, preferably dimethyl hydroxyethyl lauryl ammonium chloride. If the surfactant system comprises C₁₀-C₁₅ alkyl benzene sulfonates (LAS), the LAS is used at levels ranging from about 9% to about 25%, or from about 13% to about 25%, or from about 15% to about 23% by weight of the composition.

The surfactant system may comprise from 0% to about 7%, or from about 0.1% to about 5%, or from about 1% to about 4% by weight of the composition of a co-surfactant selected from a nonionic co-surfactant, cationic co-surfactant, anionic co-surfactant and any mixture thereof.

Non-limiting examples of nonionic co-surfactants include: C₁₂-C₁₈ alkyl ethoxylates, such as, NEODOL® nonionic surfactants from Shell; C₆-C₁₂ alkyl phenol alkoxylates wherein the alkoxylate units are a mixture of ethyleneoxy and propyleneoxy units; C₁₂-C₁₈ alcohol and C₆-C₁₂ alkyl phenol condensates with ethylene oxide/propylene oxide block alkyl polyamine ethoxylates such as PLURONIC® from BASF; C₁₄-C₂₂ mid-chain branched alcohols, BA, as discussed in U.S. Pat. No. 6,150,322; C₁₄-C₂₂ mid-chain branched alkyl alkoxylates, BAEₓ, wherein x is from 1-30, as discussed in U.S. Pat. No. 6,153,577, U.S. Pat. No. 6,020,303 and U.S. Pat. No. 6,093,856; alkylpolysaccharides as discussed in U.S. Pat. No. 4,565,647 Llenado, issued Jan. 26, 1986; specifically alkylpolyglycosides as discussed in U.S. Pat. No. 4,483,780 and U.S. Pat. No. 4,483,779; polyhydroxy detergent acid amides as discussed in U.S. Pat. No. 5,332,528; and ether capped poly(oxyalkylated) alcohol surfactants as discussed in U.S. Pat. No. 6,482,994 and WO 01/42408.

Non-limiting examples of semi-polar nonionic co-surfactants include: water-soluble amine oxides containing one alkyl moiety of from about 10 to about 18 carbon atoms and 2 moieties selected from the group consisting of alkyl moieties and hydroxyalkyl moieties containing from about 1 to about 3 carbon atoms; water-soluble phosphine oxides containing one alkyl moiety of from about 10 to about 18 carbon atoms and 2 moieties selected from the group consisting of alkyl moieties and hydroxyalkyl moieties containing from about 1 to about 3 carbon atoms; and water-soluble sulfoxides containing one alkyl moiety of from about 10 to about 18 carbon atoms and a moiety selected from the group consisting of alkyl moieties and hydroxyalkyl moieties of from about 1 to about 3 carbon atoms. See WO 01/32816, U.S. Pat. No. 4,681,704, and U.S. Pat. No. 4,133,779.

Non-limiting examples of cationic co-surfactants include: the quaternary ammonium surfactants, which can have up to 26 carbon atoms include: alkoxylate quaternary ammonium (AQA) surfactants as discussed in U.S. Pat. No. 6,136,769; dimethyl hydroxyethyl quaternary ammonium as discussed in U.S. Pat. No. 6,004,922; dimethyl hydroxyethyl lauryl ammonium chloride; polyamine cationic surfactants as discussed in WO 98/35002, WO 98/35003, WO 98/35004, WO 98/35005, and WO 98/35006; cationic ester surfactants as discussed in U.S. Pat. Nos. 4,228,042, 4,239,660 4,260,529 and U.S. Pat. No. 6,022,844; and amino surfactants as discussed in U.S. Pat. No. 6,221,825 and WO 00/47708, specifically amido propyldimethyl amine (APA).

Nonlimiting examples of anionic co-surfactants useful herein include: C₁₀-C₂₀ primary, branched chain and random alkyl sulfates (AS); C₁₀-C₁₈ secondary (2,3) alkyl sulfates; C₁₀-C₁₈ alkyl alkoxy sulfates (AEₓS) wherein x is from 1-30; C₁₀-C₁₈ alkyl alkoxy carboxylates comprising 1-5 ethoxy units; mid-chain branched alkyl sulfates as discussed in U.S. Pat. No. 6,020,303 and U.S. Pat. No. 6,060,443; mid-chain branched alkyl alkoxy sulfates as discussed in U.S. Pat. No. 6,008,181 and U.S. Pat. No. 6,020,303; modified alkylbenzene sulfonate (MLAS) as discussed in WO 99/05243, WO 99/05242 and WO 99/05244; methyl ester sulfonate (MES); alpha-olefin sulfonate (AOS); and multiple branched surfactants as described in WO 02/033976 and WO 02/033979.

The present invention may also relate to compositions comprising the inventive surfactant composition and a surfactant system comprising C₈-C₁₈ linear alkyl sulphonate surfactant and a co-surfactant.

The compositions can be in any form, namely, in the form of a liquid; a solid such as a powder, granules, agglomerate, paste, tablet, pouches, bar, gel; an emulsion; types delivered in multi-compartment containers; a spray or foam detergent; premoistened wipes the cleaning composition in combination with a nonwoven material such as that discussed in U.S. Pat. No. 6,121,165, Mackey, et al.); dry wipes (i.e., the cleaning composition in combination with a nonwoven materials, such as that discussed in U.S. Pat. No. 5,980,931. Fowler, et al.) activated with water by a consumer; and other homogeneous or multiphase consumer cleaning product forms.

In one embodiment, the cleaning composition of the present invention is a liquid or solid laundry detergent composition. In another embodiment, the cleaning composition of the present invention is a hard surface cleaning composition, preferably wherein the hard surface cleaning composition impregnates a nonwoven substrate. As used herein “impregnate” means that the hard surface cleaning composition is placed in contact with a nonwoven substrate such that at least a portion of the nonwoven substrate is penetrated by the hard surface cleaning composition, preferably the hard surface cleaning composition saturates the nonwoven substrate. The cleaning composition may also be utilized in car care compositions, for cleaning various surfaces such as hard wood, tile, ceramic, plastic, leather, metal, glass. This cleaning composition could be also designed to be used in a personal care and pet care compositions such as shampoo composition, body wash, liquid or solid soap and other cleaning composition in which surfactant comes into contact with free hardness and in all compositions that require hardness tolerant surfactant system, such as oil drilling compositions.

In another embodiment the cleaning composition is a dish cleaning composition, such as liquid hand dishwashing compositions, solid automatic dishwashing compositions, liquid automatic dishwashing compositions, and tab/unit does forms of automatic dishwashing compositions.

Quite typically, cleaning compositions herein such as laundry detergents, laundry detergent additives, hard surface cleaners, synthetic and soap-based laundry bars, fabric softeners and fabric treatment liquids, solids and treatment articles of all kinds will require several adjuncts, though certain simply formulated products, such as bleach additives, may require only, for example, an oxygen bleaching agent and a surfactant as described herein. A comprehensive list of suitable laundry or cleaning adjunct materials can be found in WO 99/05242.

Common cleaning adjuncts include builders, enzymes, polymers not discussed above, bleaches, bleach activators, catalytic materials and the like excluding any materials already defined hereinabove. Other cleaning adjuncts herein can include suds boosters, suds suppressors (antifoams) and the like, diverse active ingredients or specialized materials such as dispersant polymers (e.g., from BASF Corp. or Rohm & Haas) other than those described above, color speckles, silvercare, anti-tarnish and/or anti-corrosion agents, dyes, fillers, germicides, alkalinity sources, hydrotropes, anti-oxidants, enzyme stabilizing agents, pro-perfumes, perfumes, solubilizing agents, carriers, processing aids, pigments, and, for liquid formulations, solvents, chelating agents, dye transfer inhibiting agents, dispersants, brighteners, suds suppressors, dyes, structure elasticizing agents, fabric softeners, anti-abrasion agents, hydrotropes, processing aids, and other fabric care agents, surface and skin care agents. Suitable examples of such other cleaning adjuncts and levels of use are found in U.S. Pat. Nos. 5,576,282, 6,306,812 B1 and 6,326,348 B1.

The compositions of the invention preferably contain one or more additional detergent components selected from surfactants, enzymes, builders, alkalinity system, organic polymeric compounds, suds suppressors, soil suspension, anti-redeposition agents and corrosion inhibitors. This listing of such ingredients is exemplary only, and not by way of limitation of the types of ingredients which can be used with the near terminal-branched surfactants herein. A detailed description of additional components can be found in U.S. Pat. No. 6,020,303.

### Bleaching Compounds, Bleaching Agents, Bleach Activators, and Bleach Catalysts

The cleaning compositions herein preferably further contain bleaching agents or bleaching compositions containing a bleaching agent and one or more bleach activators. Bleaching agents will typically be at levels of from about 1 wt % to about 30 wt %, more typically from about 5 wt % to about 20 wt %, based on the total weight of the composition, especially for fabric laundering. If present, the amount of bleach activators will typically be from about 0.1 wt % to about 60 wt %, more typically from about 0.5 wt % to about 40 wt % of the bleaching composition comprising the bleaching agent-plus-bleach activator.

Examples of bleaching agents include oxygen bleach, perborate bleache, percarboxylic acid bleach and salts thereof, peroxygen bleach, persulfate bleach, percarbonate bleach, and mixtures thereof. Examples of bleaching agents are disclosed in U.S. Pat. No. 4,483,781, U.S. patent application Ser. No. 740,446, European Patent Application 0,133,354, U.S. Pat. No. 4,412,934, and U.S. Pat. No. 4,634,551.

Examples of bleach activators (e.g., acyl lactam activators) are disclosed in U.S. Pat. Nos. 4,915,854; 4,412,934; 4,634,551; 4,634,551; and 4,966,723.

### Transition Metal Bleach Catalysts

Preferably, the laundry detergent composition comprises a transition metal catalyst. Preferably, the transition metal catalyst may be encapsulated. The transition metal bleach catalyst typically comprises a transition metal ion, preferably selected from transition metal selected from the group consisting of Mn(II), Mn(III), Mn(IV), Mn(V), Fe(II), Fe(III), Fe(IV), Co(I), Co(II), Co(III), Ni(I), Ni(II), Ni(III), Cu(I), Cu(II), Cu(III), Cr(II), Cr(III), Cr(IV), Cr(V), Cr(VI), V(III), V(IV), V(V), Mo(IV), Mo(V), Mo(VI), W(IV), W(V), W(VI), Pd(II), Ru(II), Ru(III), and Ru(IV), more preferably Mn(II), Mn(III), Mn(IV), Fe(II), Fe(III), Cr(II), Cr(III), Cr(IV), Cr(V), and Cr(VI). The transition metal bleach catalyst typically comprises a ligand, preferably a macropolycyclic ligand, more preferably a cross-bridged macropolycyclic ligand. The transition metal ion is preferably coordinated with the ligand. Preferably, the ligand comprises at least four donor atoms, at least two of which are bridgehead donor atoms. Suitable transition metal bleach catalysts are described in U.S. Pat. No. 5,580,485, U.S. Pat. No. 4,430,243; U.S. Pat. No. 4,728,455; U.S. Pat. No. 5,246,621; U.S. Pat. No. 5,244,594; U.S. Pat. No. 5,284,944; U.S. Pat. No. 5,194,416; U.S. Pat. No. 5,246,612; U.S. Pat. No. 5,256,779; U.S. Pat. No. 5,280,117; U.S. Pat. No. 5,274,147; U.S. Pat. No. 5,153,161; U.S. Pat. No. 5,227,084; U.S. Pat. No. 5,114,606; U.S. Pat. No. 5,114,611, EP 549,271 A1; EP 544,490 A1; EP 549,272 A1; and EP 544,440 A2. A suitable transition metal bleach catalyst is a manganese-based catalyst, for example disclosed in U.S. Pat. No. 5,576,282. Suitable cobalt bleach catalysts are described, for example, in U.S. Pat. No. 5,597,936 and U.S. Pat. No. 5,595,967. Such cobalt catalysts are readily prepared by known procedures, such as taught for example in U.S. Pat. No. 5,597,936, and U.S. Pat. No. 5,595,967. A suitable transition metal bleach catalyst is a transition metal complex of ligand such as bispidones described in WO 05/042532 A1.

Bleaching agents other than oxygen bleaching agents are also known in the art and can be utilized herein (e.g., photoactivated bleaching agents such as the sulfonated zinc and/or aluminum phthalocyanines (U.S. Pat. No. 4,033,718), or pre-formed organic peracids such as peroxycarboxylic acid or salt thereof, or a peroxysulphonic acid or salt thereof. A suitable organic peracid is phthaloylimidoperoxycaproic acid. If used, household cleaning compositions will typically contain from about 0.025% to about 1.25%, by weight, of such bleaches, especially sulfonate zinc phthalocyanine.

### Enzymes

Enzymes are included in the present cleaning compositions for a variety of purposes, including removal of protein-based, carbohydrate-based, or triglyceride-based stains from substrates, for the prevention of refugee dye transfer in fabric laundering, and for fabric restoration. Suitable enzymes include proteases, amylases, lipases, cellulases, peroxidases, and mixtures thereof of any suitable origin, such as vegetable, animal, bacterial, fungal and yeast origin. Preferred selections are influenced by factors such as pH-activity and/or stability optima, thermostability, and stability to active detergents, builders and the like. In this respect bacterial or fungal enzymes are preferred, such as bacterial amylases and proteases, and fungal cellulases.

Enzymes are normally incorporated into detergent or detergent additive compositions at levels sufficient to provide a “cleaning-effective amount”. The term “cleaning effective amount” refers to any amount capable of producing a cleaning, stain removal, soil removal, whitening, deodorizing, or freshness improving effect on substrates such as fabrics, dishware and the like. In practical terms for current commercial preparations, typical amounts are up to about 5 mg by weight, more typically 0.01 mg to 3 mg, of active enzyme per gram of the household cleaning composition. Stated otherwise, the compositions herein will typically comprise from 0.001% to 5%, preferably 0.01%-1% by weight of a commercial enzyme preparation.

A range of enzyme materials and means for their incorporation into synthetic detergent compositions is also disclosed in WO 9307263 A; WO 9307260 A; WO 8908694 A; U.S. Pat. Nos. 3,553,139; 4,101,457; and U.S. Pat. No. 4,507,219. Enzyme materials useful for liquid detergent formulations, and their incorporation into such formulations, are disclosed in U.S. Pat. No. 4,261,868. Enzymes for use in detergents can be stabilized by various techniques. Enzyme stabilisation techniques are disclosed and exemplified in U.S. Pat. Nos. 3,600,319 and 3,519,570; EP 199,405, EP 200,586; and WO 9401532 A.

### Enzyme Stabilizing System

The enzyme-containing compositions herein may optionally also comprise from about 0.001% to about 10%, preferably from about 0.005% to about 8%, most preferably from about 0.01% to about 6%, by weight of an enzyme stabilizing system. The enzyme stabilizing system can be any stabilizing system which is compatible with the detersive enzyme. Such a system may be inherently provided by other formulation actives, or be added separately, e.g., by the formulator or by a manufacturer of detergent-ready enzymes. Such stabilizing systems can, for example, comprise calcium ion, boric acid, propylene glycol, short chain carboxylic acids, boronic acids, and mixtures thereof, and are designed to address different stabilization problems depending on the type and physical form of the detergent composition.

### Builders

Detergent builders selected from aluminosilicates and silicates are preferably included in the compositions herein, for example to assist in controlling mineral, especially calcium and/or magnesium hardness in wash water or to assist in the removal of particulate soils from surfaces. Also suitable for use herein are synthesized crystalline ion exchange materials or hydrates thereof having chain structure and a composition represented by the following general formula in an anhydride form: x(M₂O).ySiO₂.zM′O wherein M is Na and/or K, M′ is Ca and/or Mg; y/x is 0.5 to 2.0 and z/x is 0.005 to 1.0 as taught in U.S. Pat. No. 5,427,711. Detergent builders in place of or in addition to the silicates and aluminosilicates described hereinbefore can optionally be included in the compositions herein, for example to assist in controlling mineral, especially calcium and/or magnesium hardness in wash water or to assist in the removal of particulate soils from surfaces.

Builder level can vary widely depending upon end use and physical form of the composition. Built detergents typically comprise at least about 1 wt % builder, based on the total weight of the detergent. Liquid formulations typically comprise about 5 wt % to about 50 wt %, more typically 5 wt % to 35 wt % of builder to the total weight of the detergent. Granular formulations typically comprise from about 10% to about 80%, more typically 15% to 50% builder by weight of the detergent composition. Lower or higher levels of builders are not excluded. For example, certain detergent additive or high-surfactant formulations can be unbuilt.

Suitable builders herein can be selected from the group consisting of phosphates and polyphosphates, especially the sodium salts; carbonates, bicarbonates, sesquicarbonates and carbonate minerals other than sodium carbonate or sesquicarbonate; organic mono-, di-, tri-, and tetracarboxylates especially water-soluble nonsurfactant carboxylates in acid, sodium, potassium or alkanolammonium salt form, as well as oligomeric or water-soluble low molecular weight polymer carboxylates including aliphatic and aromatic types; and phytic acid. These may be complemented by borates, e.g., for pH-buffering purposes, or by sulfates, especially sodium sulfate and any other fillers or carriers which may be important to the engineering of stable surfactant and/or builder-containing detergent compositions.

### Detersive Surfactants

The detergent compositions according to the present invention preferably further comprise additional surfactants, herein also referred to as co-surfactants. It is to be understood that the mixtures of near terminal-branched surfactants prepared in the manner of the present invention may be used singly in cleaning compositions or in combination with other detersive surfactants. Typically, fully-formulated cleaning compositions will contain a mixture of surfactant types in order to obtain broad-scale cleaning performance over a variety of soils and stains and under a variety of usage conditions. One advantage of the mixtures of near terminal-branched surfactants herein is their ability to be readily formulated in combination with other known surfactant types. Nonlimiting examples of additional surfactants which may be used herein typically at levels from about 1% to about 55%, by weight, include the unsaturated sulfates, the C₁₀-C₁₈ alkyl alkoxy, C₁₀-C₁₈ alkyl alkoxy carboxylates, the C₁₀-C₁₈ glycerol ether sulfates, the C₁₀-C₁₈ alkyl polyglycosides and their corresponding sulfated polyglycosides, and C₁₀-C₁₈ alpha-sulfonated fatty acid esters. Nonionic surfactants such as the ethoxylated C₁₀-C₁₈ alcohols and alkyl phenols can also be used. If desired, other conventional surfactants such as the C₁₀-C₁₈ betaines and sulfobetaines (“sultaines”). C₁₀-C₁₈ amine oxides, and the like, can also be included in the overall compositions. The C₁₀-C₁₈ N-alkyl polyhydroxy fatty acid amides can also be used. See WO 9,206,154. Other sugar-derived surfactants include the N-alkoxy polyhydroxy fatty acid amides. The N-propyl through N-hexyl C₁₂-C₁₈ glucamides can be used for low sudsing. C₁₀-C₂₀ conventional soaps may also be used. If high sudsing is desired, the branched-chain C₁₀-C₁₆ soaps may be used. C₁₀-C₁₄ alkyl benzene sulfonates (LAS), which are often used in laundry detergent compositions, can also be used with the branched surfactants herein.

A wide range of these co-surfactants can be used in the detergent compositions of the present invention. A typical listing of anionic, nonionic, ampholytic and zwitterionic classes, and species of these co-surfactants, is given in U.S. Pat. No. 3,664,961. Amphoteric surfactants are also described in detail in “Amphoteric Surfactants, Second Edition”, E. G. Lomax, Editor (published 1996, by Marcel Dekker, Inc.)

The laundry detergent compositions of the present invention typically comprise from about 0.1% to about 35%, preferably from about 0.5% to about 15% by weight of co-surfactants, (e.g., anionic co-surfactants, nonionic co-surfactants, cationic co-surfactants).

### Amine-Neutralized Anionic Surfactants

Anionic surfactants of the present invention and adjunct anionic cosurfactants may be neutralized by amines or alkanolamines, and alkanolamines are preferred. Suitable non-limiting examples including monoethanolamine, triethanolamine, and other alkanolamines known in the art.

### Polymeric Soil Release Agent

Known polymeric soil release agents, hereinafter “SRA” or “SRA's”, can optionally be employed in the present detergent compositions. If utilized, SRA's will generally comprise from 0.01% to 10.0%, typically from 0.1% to 5%, preferably from 0.2% to 3.0% by weight, of the composition.

Preferred SRA's typically have hydrophilic segments to hydrophilize the surface of hydrophobic fibers such as polyester and nylon, and hydrophobic segments to deposit upon hydrophobic fibers and remain adhered thereto through completion of washing and rinsing cycles thereby serving as an anchor for the hydrophilic segments. This can enable stains occurring subsequent to treatment with SRA to be more easily cleaned in later washing procedures.

SRA's can include, for example, a variety of charged, e.g., anionic or even cationic see U.S. Pat. No. 4,956,447), as well as noncharged monomer units and structures may be linear, branched or even star-shaped. They may include capping moieties which are especially effective in controlling molecular weight or altering the physical or surface-active properties, Structures and charge distributions may be tailored for application to different fiber or textile types and for varied detergent or detergent additive products. Examples of SRAs are described in U.S. Pat. Nos. 4,968,451; 4,711,730; 4,721,580; 4,702,857; 4,877,896; 3,959,230; 3,893,929; 4,000,093; 5,415,807; 4,201,824; 4,240,918; 4,525,524; 4,201,824; 4,579,68|; and 4,787,989; European Patent Application 0 219 048; 279,134 A; 457,205 A; and DE 2,335,044, all of which are.

### Clay Soil Removal/Anti-Redeposition Agents

The compositions of the present invention can also optionally contain water-soluble ethoxylated amines having clay soil removal and antiredeposition properties. Granular detergent compositions which contain these compounds typically contain from about 0.01% to about 10.0% by weight of the water-soluble ethoxylates amines; liquid detergent compositions typically contain about 0.01% to about 5% by weight.

Exemplary clay soil removal and antiredeposition agents are described in U.S. Pat. Nos. 4,597,898; 548,744; 4,891,160; European Patent Application Nos. 111,965; 111,984; 112,592; and WO 95/32272, which are all.

### Polymeric Dispersing Agents

Polymeric dispersing agents can advantageously be utilized at levels from about 0.1% to about 7%, by weight, in the compositions herein, especially in the presence of zeolite and/or layered silicate builders. Suitable polymeric dispersing agents include polymeric polycarboxylates and polyethylene glycols, although others known in the art can also be used. It is believed, though it is not intended to be limited by theory, that polymeric dispersing agents enhance overall detergent builder performance, when used in combination with other builders (including lower molecular weight polycarboxylates) by crystal growth inhibition, particulate soil release peptization, and anti-redeposition. Examples of polymeric dispersing agents are found in U.S. Pat. No. 3,308,067, European Patent Application No. 66915, EP 193,360, and EP 193,360, which are

### Alkoxylated Polyamine Polymers

Soil suspension, grease cleaning, and particulate cleaning polymers may include the alkoxylated polyamines. Such materials include but are not limited to ethoxylated polyethyleneimine, ethoxylated hexamethylene diamine, and sulfated versions thereof. A useful example is 600 g/mol polyethyleneimine core ethoxylated to 20 EO groups per NH and is available from BASF.

### Brightener

Any optical brighteners or other brightening or whitening agents known in the art can be incorporated at levels typically from about 0.01% to about 1.2%, by weight, into the cleaning compositions herein. Commercial optical brighteners which may be useful in the present invention can be classified into subgroups, which include, but are not necessarily limited to, derivatives of stilbene, pyrazoline, coumarin, carboxylic acid, methinecyanines, dibenzothiophene-5,5-dioxide, azoles, 5- and 6-membered-ring heterocycles, and other miscellaneous agents. Examples of such brighteners are disclosed in “The Production and Application of Fluorescent Brightening Agents”, M. Zahradnik, Published by John Wiley & Sons, New York (1982). Specific examples of optical brighteners which are useful in the present compositions are those identified in U.S. Pat. No. 4,790,856 and U.S. Pat. No. 3,646,015, which are

### Fabric Hueing Agents

The compositions of the present invention my include fabric hueing agents. Non-limiting examples include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof. In another aspect, suitable polymeric dyes include polymeric dyes selected from the group consisting of fabric-substantive colorants sold under the name of Liquitint® (Milliken, Spartanburg, S.C., USA), dye-polymer conjugates formed from at least one reactive dye and a polymer selected from the group consisting of polymers comprising a moiety selected from the group consisting of a hydroxyl moiety, a primary amine moiety, a secondary amine moiety, a thiol moiety and mixtures thereof. In still another aspect, suitable polymeric dyes include polymeric dyes selected from the group consisting of Liquitint® (Milliken, Spartanburg, S.C., USA) Violet Conn., carboxymethyl cellulose (CMC) conjugated with a reactive blue, reactive violet or reactive red dye such as CMC conjugated with C.I. Reactive Blue 19, sold by Megazyme, Wicklow, Ireland under the product name AZO-CM-CELLULOSE, product code S-ACMC, alkoxylated triphenyl-methane polymeric colorants, alkoxylated thiophene polymeric colorants, and mixtures thereof.

### Dye Transfer Inhibiting Agents

The compositions of the present invention may also include one or more materials effective for inhibiting the transfer of dyes from one fabric to another during the cleaning process. Generally, such dye transfer inhibiting agents include polyvinyl pyrrolidone polymers, polyamine N-oxide polymers, copolymers of N-vinylpyrrolidone and N-vinylimidazole, manganese phthalocyanine, peroxidases, and mixtures thereof. If used, these agents typically comprise from about 0.01% to about 10% by weight of the composition, preferably from about 0.01% to about 5%, and more preferably from about 0.05% to about 2%.

### Chelating Agents

The detergent compositions herein may also optionally contain one or more iron and/or manganese chelating agents. Such chelating agents can be selected from the group consisting of amino carboxylates, amino phosphonates, polyfunctionally-substituted aromatic chelating agents and mixtures therein. If utilized, these chelating agents will generally comprise from about 0.1% to about 15% by weight of the detergent compositions herein. More preferably, if utilized, the chelating agents will comprise from about 0.1% to about 3.0% by weight of such compositions.

### Suds Suppressors

Compounds for reducing or suppressing the formation of suds can be incorporated into the compositions of the present invention. Suds suppression can be of particular importance in the so-called “high concentration cleaning process” as described in U.S. Pat. Nos. 4,489,455 and 4,489,574, and in front-loading European-style washing machines.

A wide variety of materials may be used as suds suppressors, and suds suppressors are well known to those skilled in the art. See, for example, Kirk Othmer Encyclopedia of Chemical Technology, Third Edition, Volume 7, pages 430-447 (John Wiley & Sons, Inc., 1979). Examples of suds supressors include monocarboxylic fatty acid and soluble salts therein, high molecular weight hydrocarbons such as paraffin, fatty acid esters (e.g., fatty acid triglycerides), fatty acid esters of monovalent alcohols, aliphatic C₁₈-C₄₀ ketones (e.g., stearone), N-alkylated amino triazines, waxy hydrocarbons preferably having a melting point below about 100° C., silicone suds suppressors, and secondary alcohols. Suds supressors are described in U.S. Pat. Nos. 2,954,347; 4,265,779; 4,265,779; 3,455,839; 3,933,672; 4,652,392; 4,978,471;. 4,983.316; 5,288,431; 4,639,489; 4,749,740; and 4,798,679; 4,075,118; European Patent Application No. 89307851.9; EP 150,872; and DOS 2,124,526 which are all

For any detergent compositions to be used in automatic laundry washing machines, suds should not form to the extent that they overflow the washing machine, Suds suppressors, when utilized, are preferably present in a “suds suppressing amount. By “suds suppressing amount” is meant that the formulator of the composition can select an amount of this suds controlling agent that will sufficiently control the suds to result in a low-sudsing laundry detergent for use in automatic laundry washing machines.

The compositions herein will generally comprise from 0% to about 10% of suds suppressor. When utilized as suds suppressors, monocarboxylic fatty acids, and salts therein, will be present typically in amounts up to about 5%, by weight, of the detergent composition. Preferably, from about 0.5% to about 3% of fatty monocarboxylate suds suppressor is utilized. Silicone suds suppressors are typically utilized in amounts up to about 2.0%, by weight, of the detergent composition, although higher amounts may be used. Monostearyl phosphate suds suppressors are generally utilized in amounts ranging from about 0.1% to about 2%, by weight, of the composition. Hydrocarbon suds suppressors are typically utilized in amounts ranging from about 0.01% to about 5.0%, although higher levels can be used. The alcohol suds suppressors are typically used at 0.2%-3% by weight of the finished compositions.

### Structurant/Thickeners

Structured liquids can either be internally structured, whereby the structure is formed by primary ingredients (e.g. surfactant material) and/or externally structured by providing a three dimensional matrix structure using secondary ingredients (e.g. polymers, clay and/or silicate material). The composition may comprise a structurant, preferably from 0.01 wt % to 5 wt %, from 0.1 wt % to 2.0 wt % structurant. The structurant is typically selected from the group consisting of diglycerides and triglycerides, ethylene glycol distearate, microcrystalline cellulose, cellulose-based materials, microfiber cellulose, biopolymers, xanthan gum, gellan gum, and mixtures thereof. A suitable structurant includes hydrogenated castor oil, and non-ethoxylated derivatives thereof. A suitable structurant is U.S. Pat. No. 6,855,680, such structurants have a thread-like structuring system having a range of aspect ratios. Other suitable structurants and the processes for making them are described in WO2010/034736.

### Alkoxylated Polycarboxylates

Alkoxylated polycarboxylates such as those prepared from polyacrylates are useful herein to provide additional grease removal performance. Such materials are described in WO 91/08281 and PCT 90/01815. Chemically, these materials comprise polyacrylates having one ethoxy side-chain per every 7-8 acrylate units. The side-chains are of the formula —(CH₂CH₂O)ₘ(CH₂)ₙCH₃ wherein m is 2-3 and n is 6-12. The side-chains are ester-linked to the polyacrylate “backbone” to provide a “comb” polymer type structure. The molecular weight can vary, but is typically in the range of about 2000 to about 50.000. Such alkoxylated polycarboxylates can comprise from about 0.05% to about 10%, by weight, of the compositions herein.

### Amphilic Graft Co-Polymer

The near-terminal branched surfactants of the present invention, and their mixtures with other cosurfactants and other adjunct ingredients, are particularly suited to be used with an amphilic graft co-polymer, preferably the amphilic graft co-polymer comprises (i) polyethyelene glycol backbone; and (ii) and at least one pendant moiety selected from polyvinyl acetate, polyvinyl alcohol and mixtures thereof. A preferred amphilic graft co-polymer is Sokalan HP22, supplied from BASF.

### Fabric Softeners

Various through-the-wash fabric softeners, especially the impalpable smectite clays of U.S. Pat. No. 4,062,647, as well as other softener clays known in the art, can optionally be used typically at levels of from about 0.5% to about 10% by weight in the present compositions to provide fabric softener benefits concurrently with fabric cleaning. Clay softeners can be used in combination with amine and cationic softeners as disclosed, for example, in U.S. Pat. No. 4,375,416, and U.S. Pat. No. 4,291,071, which are.

### Perfumes

Perfumes and perfumery ingredients useful in the present compositions and processes comprise a wide variety of natural and synthetic chemical ingredients, including, but not limited to, aldehydes, ketones, esters, and the like. Also included are various natural extracts and essences which can comprise complex mixtures of ingredients, such as orange oil, lemon oil, rose extract, lavender, musk, patchouli, balsamic essence, sandalwood oil, pine oil, cedar, and the like. Finished perfumes can comprise extremely complex mixtures of such ingredients. Finished perfumes typically comprise from about 0.01% to about 2%, by weight, of the detergent compositions herein, and individual lay softeners can be used in combination with amine and cationic softeners perfumery ingredients can comprise from about 0.0001% to about 90% of a finished perfume composition.

### Other Ingredients

A wide variety of other ingredients useful in the cleaning compositions can be included in the compositions herein, including other active ingredients, carriers, hydrotropes, processing aids, dyes or pigments, solvents for liquid formulations, solid fillers for bar compositions, etc. If high sudsing is desired, suds boosters such as the C₁₀-C₁₆ alkanolamides can be incorporated into the compositions, typically at 1%-10% levels. The C₁₀-C₁₄ monoethanol and diethanol amides illustrate a typical class of such suds boosters. Use of such suds boosters with high sudsing adjunct surfactants such as the amine oxides, betaines and sultaines noted above is also advantageous. If desired, water-soluble magnesium and/or calcium salts such as MgCl₂, MgSO₄, CaCl₂, CaSO₄ and the like, can be added at levels of, typically, 0.1%-2%, to provide additional suds and to enhance grease removal performance.

Various detersive ingredients employed in the present compositions optionally can be further stabilized by absorbing said ingredients onto a porous hydrophobic substrate, then coating said substrate with a hydrophobic coating. Preferably, the detersive ingredient is admixed with a surfactant before being absorbed into the porous substrate. In use, the detersive ingredient is released from the substrate into the aqueous washing liquor, where it performs its intended detersive function.

Liquid detergent compositions can contain water and other solvents as carriers. Low molecular weight primary or secondary alcohols exemplified by methanol, ethanol, propanol, and isopropanol are suitable. Monohydric alcohols are preferred for solubilizing surfactant, but polyols such as those containing from 2 to about 6 carbon atoms and from 2 to about 6 hydroxy groups (e.g., 1,3-propanediol, ethylene glycol, glycerine, and 1,2-propanediol) can also be used. The compositions may contain from 5% to 90%, typically 10% to 50% by weight of such carriers.

The cleaning compositions herein will preferably be formulated such that, during use in aqueous cleaning operations, the wash water will have a pH of between about 6.5 and about 11, preferably between about 7.5 and 10.5. Liquid dishwashing product formulations preferably have a pH between about 6.8 and about 9.0. Laundry products are typically at pH 9-11. Techniques for controlling pH at recommended usage levels include the use of buffers, alkalis, acids, etc., and are well known to those skilled in the art.

### Form of the Compositions

The compositions in accordance with the invention can take a variety of physical forms including granular, tablet, bar and liquid forms. Also included are a sachet, a two in one pouch containing both solid and liquid compartments, a tablet, The compositions are particularly the so-called concentrated granular detergent compositions adapted to be added to a washing machine by means of a dispensing device placed in the machine drum with the soiled fabric load.

### Compacted Liquid or Powder Detergents

The near-terminal branched surfactants of the present invention, and their mixtures with other cosurfactants and other adjunct ingredients, are particularly suited to compact detergent formulations. For liquid detergents, the composition preferably comprises less than 20 wt %, or less than 10 wt %, or less than 5 wt %, or less than 4 wt % or less than 3 wt % free water, or less than 2 wt % free water, or less than 1 wt % free water, and may even be anhydrous, typically comprising no deliberately added free water. Free water is typically measured using Karl Fischer titration. 2 g of the laundry detergent composition is extracted into 50 ml dry methanol at room temperature for 20 minutes and analyse 1 of the methanol by Karl Fischer titration. For powder detergents, the amount of filler (sodium sulfate, sodium chloride, clay, or other inert solid ingredients) preferably comprises less than 20 wt %, or less than 10 wt %, or less than 5 wt %, or less than 4 or less than 3 wt % free water, or less than 2 wt % free water, or less than 1 wt % filler.

### Methods of Using Such Household Cleaning Products

The present invention includes a method for cleaning a targeted surface. As used herein “targeted surface” may include such surfaces such as fabric, dishes, glasses, and other cooking surfaces, hard surfaces, hair or skin. As used herein “hard surface” includes hard surfaces being found in a typical home such as hard wood, tile, ceramic, plastic, leather, metal, glass. Such method includes the steps of contacting the composition comprising the modified alcohol compound, in neat form or diluted in wash liquor, with at least a portion of a targeted surface then optionally rinsing the targeted surface. Preferably the targeted surface is subjected to a washing step prior to the aforementioned optional rinsing step. For purposes of the present invention, washing includes, but is not limited to, scrubbing, wiping and mechanical agitation.

As will be appreciated by one skilled in the art, the cleaning compositions of the present invention are ideally suited for use in home care (hard surface cleaning compositions) and/or laundry applications.

The composition solution pH is chosen to be the most complimentary to a target surface to be cleaned spanning broad range of pH, from about 5 to about 11. For personal care such as skin and hair cleaning pH of such composition preferably has a pH from about 5 to about 8 for laundry cleaning compositions pH of from about 8 to about 10. The compositions are preferably employed at concentrations of from about 200 ppm to about 10,000 ppm in solution. The water temperatures preferably range from about 5° C. to about 100° C.

For use in laundry cleaning compositions, the compositions are preferably employed at concentrations from about 100 ppm to about 10000 ppm in solution (or wash liquor). The water temperatures preferably range from about 5° C. to about 60° C. The water to fabric ratio is preferably from about 1:1 to about 20:1.

The method may include the step of contacting a nonwoven substrate impregnated with an embodiment of the composition of the present invention As used herein “nonwoven substrate” can comprise any conventionally fashioned nonwoven sheet or web having suitable basis weight, caliper (thickness), absorbency and strength characteristics. Examples of suitable commercially available nonwoven substrates include those marketed under the tradename SONTARA® by DuPont and POLYWEB® by James River Corp.

In addition, another advantage of the compositions herein is their desirable performance in cold water. The invention herein includes methods for laundering of fabrics at reduced wash temperatures. This method of laundering fabric comprises the step of contacting a laundry detergent composition to water to form a wash liquor, and laundering fabric in said wash liquor, wherein the wash liquor has a temperature of above 0° C. to 20° C., preferably to 19° C., or to 18° C. or to 17° C., or to 16° C., or to 15° C. or to 14° C., or to 13° C., or to 12″C, or to 11° C., or to 10° C., or to 9° C., or to 8° C., or to 7° C. or to 6° C., or even to 5° C. The fabric may be contacted to the water prior to, or after, or simultaneous with, contacting the laundry detergent composition with water.

A further method of use of the materials of the present invention involves pretreatment of stains prior to laundering.

### Hand and Machine Dishwashing Methods

As will be appreciated by one skilled in the art, the cleaning compositions of the present invention are ideally suited for use in liquid dish cleaning compositions. The method for using a liquid dish composition of the present invention comprises the steps of contacting soiled dishes with an effective amount, typically from about 0.5 ml to about 20 ml. (per 25 dishes being treated) of the liquid dish cleaning composition of the present invention diluted in water.

Any suitable methods for machine washing or cleaning soiled tableware, particularly soiled silverware are envisaged. A preferred liquid hand dishwashing method involves either the dissolution of the detergent composition into a recepticle containing water, or by the direct application of the liquid hand dishwashing detergent composition onto soiled dishware. A preferred machine dishwashing method comprises treating soiled articles selected from crockery, glassware, hollowware, silverware and cutlery and mixtures thereof, with an aqueous liquid having dissolved or dispensed therein an effective amount of a machine dishwashing composition in accord with the invention. By an effective amount of the machine dishwashing composition it is meant from 8 g to 60 g of product dissolved or dispersed in a wash solution of volume from 3 to 10 liters, as are typical product dosages and wash solution volumes commonly employed in conventional machine dishwashing methods.

### Personal Care Products

Human hair becomes dry and/or damaged due to the surrounding environment, styling, drying, and/or coloring or otherwise chemically treating the hair.

A variety of approaches have been developed to condition the hair. A common method of providing conditioning benefit is through the use of hair care compositions containing conditioning agents such as cationic surfactants and polymers, high melting point fatty compounds, low melting point oils, silicone compounds, and mixtures thereof. Silicones are often used as a conditioning active for a number of hair care compositions.

Based on the foregoing, there is a need for personal care compositions which can provide conditioning benefits to hair which can be used in combination with silicone to maximize the conditioning activity. Additionally, there is a need for personal care compositions that can deliver a conditioning benefit to damaged hair, which has previously been difficult to condition using traditional conditioning actives. There is also a need for the cleaning compounds with properties that lend themselves to higher tolerance to precipitation with calcium and magnesium in hard water. There is also a continued need for personal care compositions that demonstrate excellent rinsability, particularly fast rinsability. Furthermore, there is a need for the cleaning compounds that provide improved cleaning in the cooler wash temperatures. There is also a need for efficient cleaning compounds that also have improved biodegradability. Moreover, there is a need to provide cleaning compounds from non petroleum sources for future sustainability.

The novel microbially produced fatty alcohols and derivatives according to the present invention can solve such problems. In certain embodiments, the fatty alcohols and derivatives can be provided in an amount of from about 0.1% to about 10% of the total weight of the personal care composition, from about 0.5% to about 5%, from about 0.8% to about 2.5%, or from about 0.9% to about 1.5% of the total weight of the composition.

In an embodiment, the personal care composition further comprises a component selected from the group consisting of thickeners; glossing and shine-imparting agents; dyes or color-imparting agents; particles; glitter or colored particles; and mixtures thereof. Examples of these components are provided below.

In an embodiment, the personal care composition is a multiphase composition comprising visually distinct phases, wherein the visually distinct phases form a pattern selected from the group consisting of striped, swirled, spiral, marbled, and mixtures thereof.

In an embodiment, the personal care composition comprises at least one silicone comprising an amine group, preferably a terminal aminosilicone, more preferably an amodimethicone. Other silicones are described below.

In an embodiment, the personal care composition is a shampoo composition and further comprises at least one surfactant compound; and at least one cosmetically acceptable carrier, Surfactants are described herein.

In an embodiment, the personal care composition is a hair conditioning composition and further comprises at least one cosmetically acceptable carrier, and at least one further compound selected from the group consisting of cationic polymers, high melting point fatty compounds. Cationic polymers and fatty compounds are described below.

In an embodiment, the personal care composition is a hair styling composition and further comprises at least one hair fixing polymer and at least one cosmetically acceptable carrier. The hair styling composition may be in a form selected from the group consisting of mousses, hairsprays, pump sprays, gels, foams, and waxes. The hair styling composition may further comprise a propellant wherein the propellant is selected from the group consisting of propane, butane, and nitrogen gas. Other propellants are also suitable, for example 1,1-difluoroethane, compressed air, isobutene, dimethylether. The hair styling composition comprises a hair fixing polymer selected from the group consisting of anionic polymers, cationic polymers, nonionic polymers, zwitterionic polymers, amphoteric polymers, and mixtures thereof. In a preferred embodiment, the hair styling composition comprises a hair fixing polymer which comprises acrylate groups.

The personal care compositions of the present inventions may include the following components:

### A. Surfactant

The composition of the present invention may include a surfactant. The surfactant component comprises anionic surfactant, cationic surfactant, zwitterionic or amphoteric surfactant, or a combination thereof. The concentration of the anionic surfactant component in the composition should be sufficient to provide the desired cleaning and lather performance, and generally range from about 5% to about 50%.

Preferred anionic surfactants suitable for use in the compositions are the alkyl and alkyl ether sulfates. Other suitable anionic surfactants are the water soluble salts of organic, sulfuric acid reaction products conforming to the formula [R1 SO3 M] where R1 is a straight or branched chain, saturated, aliphatic hydrocarbon radical having from about 8 to about 24, preferably about 10 to about 18, carbon atoms; and M is a cation selected from the group including sodium (Na⁺), potassium (K⁺), ammonium (NH₄⁺), triethylammonium (NEt₃H⁺), and magnesium (Mg²⁺). Still other suitable anionic surfactants are the reaction products of fatty acids esterified with isethionic acid and neutralized with sodium hydroxide where, for example, the fatty acids are derived from coconut oil or palm kernel oil; sodium or potassium salts of fatty acid amides of methyl tauride in which the fatty acids, for example, are derived from coconut oil or palm kernel oil. Other similar anionic surfactants are described in U.S. Pat. Nos. 2,486,921; 2,486,922; and 2,396,278.

Other anionic surfactants suitable for use in the compositions are the succinnates, examples of which include disodium N-octadecylsulfosuccinnate; disodium lauryl sulfosuccinate; diammonium lauryl sulfosuccinate; tetrasodium N-(1,2-dicarboxyethyl)-N-octadecylsulfosuccinnate; diamyl ester of sodium sulfosuccinic acid; dihexyl ester of sodium sulfosuccinic acid; and dioctyl esters of sodium sulfosuccinic acid.

Other suitable anionic surfactants include olefin sulfonates having about 10 to about 24 carbon atoms. In addition to the true alkene sulfonates and a proportion of hydroxy alkanesulfonates, the olefin sulfonates can contain minor amounts of other materials, such as alkene disulfonates depending upon the reaction conditions, proportion of reactants, the nature of the starting olefins and impurities in the olefin stock and side reactions during the sulfonation process. A non limiting example of such an alpha olefin sulfonate mixture is described in U.S. Pat. No. 3,332,880.

Another class of anionic surfactants suitable for use in the compositions are the beta-alkyloxy alkane sulfonates. These surfactants conform to the formula

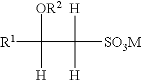

where R1 is a straight chain alkyl group having from about 6 to about 20 carbon atoms, R² is a lower alkyl group having from about 1 to about 3 carbon atoms, preferably 1 carbon atom, and M is a water soluble cation as described hereinbefore.

Non limiting examples of other anionic, zwitterionic, amphoteric or optional additional surfactants suitable for use in the compositions are described in McCutcheon's, Emulsifiers and Detergents, 1989 Annual, published by M. C. Publishing Co., and U.S. Pat. Nos. 3,929,678, 2,658,072; 2,438,091; 2,528,378.

### B. Cationic Surfactant System

The composition of the present invention may comprise a cationic surfactant system. The cationic surfactant system can be one cationic surfactant or a mixture of two or more cationic surfactants. If present, the cationic surfactant system is included in the composition at a level by weight of from about 0.1% to about 10%, preferably from about 0.5% to about 8%, more preferably from about 1% to about 5%, still more preferably from about 1.4% to about 4%, in view of balance among ease-to-rinse feel, rheology and wet conditioning benefits.

A variety of cationic surfactants including mono- and di-alkyl chain cationic surfactants can be used in the compositions of the present invention. Among them, preferred are mono-alkyl chain cationic surfactants in view of providing desired gel matrix and wet conditioning benefits. The mono-alkyl cationic surfactants are those having one long alkyl chain which has from 12 to 22 carbon atoms, preferably from 16 to 22 carbon atoms, more preferably C18-22 alkyl group, in view of providing balanced wet conditioning benefits. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms. Such mono-alkyl cationic surfactants include, for example, mono-alkyl quaternary ammonium salts and mono-alkyl amines. Mono-alkyl quaternary ammonium salts include, for example, those having a non-functionalized long alkyl chain. Mono-alkyl amines include, for example, mono-alkyl amidoamines and salts thereof.

Mono-long alkyl quaternized ammonium salts useful herein are those having the formula (II):

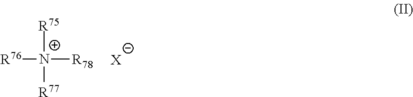

wherein one of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. Preferably, one of R⁷⁵, R⁷⁶. R⁷⁷ and R⁷⁸ is selected from an alkyl group of from 12 to 30 carbon atoms, more preferably from 16 to 22 carbon atoms, still more preferably from 18 to 22 carbon atoms, even more preferably 22 carbon atoms; the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.

Examples of preferred mono-long alkyl quaternized ammonium salt cationic surfactants include: behenyl trimethyl ammonium salt; stearyl trimethyl ammonium salt; cetyl trimethyl ammonium salt; and hydrogenated tallow alkyl trimethyl ammonium salt. Among them, highly preferred are behenyl trimethyl ammonium salt and stearyl trimethyl ammonium salt. In another embodiment, these are selected from the group consisting of behenyltrimmonium chloride, behenyltrimmonium methosulfate, cetyltrimethyl ammonium chloride, stearyltrimethyl ammonium chloride, dicetyldimethyl ammonium chloride, and distearyldimethyl ammonium chloride and mixtures thereof.

Mono-alkyl amines are also suitable as cationic surfactants. Primary, secondary, and tertiary fatty amines are useful. Particularly useful are tertiary amido amines having an alkyl group of from about 12 to about 22 carbons. Exemplary tertiary amido amines include: stearamidopropyldimethylamine, stearamidopropyldiethyl amine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethyl amine, arachidamidoethyldimethylamine, diethylaminoethylstearamide. Useful amines in the present invention are disclosed in U.S. Pat. No. 4,275,055. These amines can also be used in combination with acids such as l-glutamic acid, lactic acid, hydrochloric acid, malic acid, succinic acid, acetic acid, fumaric acid, tartaric acid, citric acid, l-glutamic hydrochloride, maleic acid, and mixtures thereof; more preferably l-glutamic acid, lactic acid, citric acid. The amines herein are preferably partially neutralized with any of the acids at a molar ratio of the amine to the acid of from about 1:0.3 to about 1:2, more preferably from about 1:0.4 to about 1:1.

Although the mono-alkyl chain cationic surfactants are preferred, other cationic surfactants such as di-alkyl chain cationic surfactants may also be used alone, or in combination with the mono-alkyl chain cationic surfactants. Such di-alkyl chain cationic surfactants include, for example, dialkyl (14-18) dimethyl ammonium chloride, ditallow alkyl dimethyl ammonium chloride, dihydrogenated tallow alkyl dimethyl ammonium chloride, distearyl dimethyl ammonium chloride, and dicetyl dimethyl ammonium chloride.

### C. High Melting Point Fatty Compound

The composition of the present invention may include a high melting point fatty compound. The high melting point fatty compound useful herein has a melting point of 25° C. or higher, and is selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than 25° C. Such compounds of low melting point are not intended to be included in this section. Non-limiting examples of the high melting point compounds are found in International Cosmetic Ingredient Dictionary, Fifth Edition, 1993, and CTFA Cosmetic Ingredient Handbook, Second Edition, 1992.

Among a variety of high melting point fatty compounds, fatty alcohols are preferably used in the composition of the present invention. The fatty alcohols useful herein are those having from about 14 to about 30 carbon atoms, preferably from about 16 to about carbon atoms. These fatty alcohols are saturated and can be straight or branched chain alcohols. Preferred fatty alcohols include, for example, cetyl alcohol, stearyl alcohol, behenyl alcohol, and mixtures thereof.

High melting point fatty compounds of a single compound of high purity are preferred. Single compounds of pure fatty alcohols selected from the group of pure cetyl alcohol, stearyl alcohol, and behenyl alcohol are highly preferred. By “pure” herein, what is meant is that the compound has a purity of at least about 90%, preferably at least about 95%. These single compounds of high purity provide good rinsability from the hair when the consumer rinses off the composition.

The high melting point fatty compound is included in the composition at a level of from about 0.1% to about 40%, preferably from about 1% to about 30%, more preferably from about 1.5% to about 16% by weight of the composition, from about 1.5% to about 8% in view of providing improved conditioning benefits such as slippery feel during the application to wet hair, softness and moisturized feel on dry hair.

### D. Cationic Polymers

The compositions of the present invention may contain a cationic polymer.

Concentrations of the cationic polymer in the composition typically range from about 0.05% to about 3%, in another embodiment from about 0.075% to about 2.0%, and in yet another embodiment from about 0.1% to about 1.0%. Suitable cationic polymers will have cationic charge densities of at least about 0.5 meq/gm, in another embodiment at least about 0.9 meg/gm, in another embodiment at least about 1.2 meg/gm, in yet another embodiment at least about 1.5 meg/gm, but in one embodiment also less than about 7 meg/gm, and in another embodiment less than about 5 meg/gm, at the pH of intended use of the composition, which pH will generally range from about pH 3 to about pH 9, in one embodiment between about pH 4 and about pH 8. Herein, “cationic charge density” of a polymer refers to the ratio of the number of positive charges on the polymer to the molecular weight of the polymer. The average molecular weight of such suitable cationic polymers will generally be between about 10,000 and 10 million, in one embodiment between about 50,000 and about 5 million, and in another embodiment between about 100,000 and about 3 million.

Suitable cationic polymers for use in the compositions of the present invention contain cationic nitrogen-containing moieties such as quaternary ammonium or cationic protonated amino moieties. The cationic protonated amines can be primary, secondary, or tertiary amines (preferably secondary or tertiary), depending upon the particular species and the selected pH of the composition. Any anionic counterions can be used in association with the cationic polymers so long as the polymers remain soluble in water, in the composition, or in a coacervate phase of the composition, and so long as the counterions are physically and chemically compatible with the essential components of the composition or do not otherwise unduly impair product performance, stability or aesthetics. Non limiting examples of such counterions include halides (e.g., chloride, fluoride, bromide, iodide), sulfate and methylsulfate.

Non limiting examples of such polymers are described in the CTFA Cosmetic Ingredient Dictionary, 3rd edition, edited by Estrin, Crosley, and Haynes, (The Cosmetic, Toiletry, and Fragrance Association, Inc., Washington, D.C. (1982)).

Non limiting examples of suitable cationic polymers include copolymers of vinyl monomers having cationic protonated amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone or vinyl pyrrolidone.

Suitable cationic protonated amino and quaternary ammonium monomers, for inclusion in the cationic polymers of the composition herein, include vinyl compounds substituted with dialkylaminoalkyl acrylate, dialkylaminoalkyl methacrylate, monoalkylaminoalk acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts.

Other suitable cationic polymers for use in the compositions include copolymers of 1-vinyl-2-pyrrolidone and 1-vinyl-3-methylimidazolium salt (e.g., chloride salt) (referred to in the industry by the Cosmetic, Toiletry, and Fragrance Association, “CTFA”, as polyquarternium-16); copolymers of 1-vinyl-2-pyrrolidone and dimethylaminoethyl methacrylate (referred to in the industry by CTFA as Polyquaternium-11); cationic diallyl quaternary ammonium-containing polymers, including, for example, dimethyldiallylammonium chloride homopolymer, copolymers of acrylamide and dimethyldiallylammonium chloride (referred to in the industry by CTFA as Polyquaternium 6 and Polyquaternium 7, respectively); amphoteric copolymers of acrylic acid including copolymers of acrylic acid and dimethyldiallylammonium chloride (referred to in the industry by CTFA as Polyquaternium 22), terpolymers of acrylic acid with dimethyldiallylammonium chloride and acrylamide (referred to in the industry by CTFA as Polyquaternium 39), and terpolymers of acrylic acid with methacrylamidopropyl trimethylammonium chloride and methylacrylate (referred to in the industry by CTFA as Polyquaternium 47). Preferred cationic substituted monomers are the cationic substituted dialkylaminoalkyl acrylamides, dialkylaminoalkyl methacrylamides, and combinations thereof. These preferred monomers conform the to the formula

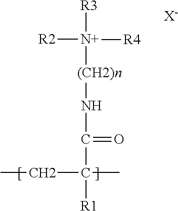

wherein R1 is hydrogen, methyl or ethyl; each of R2, R3 and R4 are independently hydrogen or a short chain alkyl haing from about 1 to about 8 carbon atoms, preferably from about 1 to about 5 carbon atoms, more preferably from about 1 to about 2 carbon atoms; n is an integer having a value of from about 1 to about 8, preferably from about 1 to about 4; and X is a counterion. The nitrogen attached to R2, R3 and R4 may be a protonated amine (primary, secondary or tertiary), but is preferably a quaternary ammonium wherein each of R2, R3 and R4 are alkyl groups a non limiting example of which is polymethyacrylamidopropyl trimonium chloride, available under the trade name Polycare 133, from Rhone-Poulenc, Cranberry, N.J., U.S.A.

Other suitable cationic polymers for use in the composition include polysaccharide polymers, such as cationic cellulose derivatives and cationic starch derivatives. Suitable cationic polysaccharide polymers include those which conform to the formula

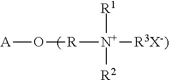

wherein A is an anhydroglucose residual group, such as a starch or cellulose anhydroglucose residual; R is an alkylene oxyalkylene, polyoxyalkylene, or hydroxyalkylene group, or combination thereof; R1, R2, and R3 independently are alkyl, aryl alkylaryl, arylalkyl, alkoxyalkyl, or alkoxyaryl groups, each group containing up to about 18 carbon atoms, and the total number of carbon atoms for each cationic moiety (i.e., the sum of carbon atoms in R1, R2 and R3) preferably being about 20 or less; and X is an anionic counterion as described in hereinbefore.

Preferred cationic cellulose polymers are salts of hydroxyethyl cellulose reacted with trimethyl ammonium substituted epoxide, referred to in the industry (CTRA) as Polyquaternium 10 and available from Amerchol Corp. (Edison, N.J., USA) in their Polymer LR, JR, and KG series of polymers. Other suitable types of cationic cellulose includes the polymeric quaternary ammonium salts of hydroxyethyl cellulose reacted with lauryl dimethyl ammonium-substituted epoxide referred to in the industry (CTFA) as Polyquaternium 24. These materials are available from Amerchol Corp, under the tradename Polymer LM-200.

Other suitable cationic polymers include cationic guar gum derivatives, such as guar hydroxypropyltrimonium chloride, specific examples of which include the Jaguar series commercially available from Rhone-Poulenc Incorporated and the N-Hance series commercially available from Aqualon Division of Hercules, Inc. Other suitable cationic polymers include quaternary nitrogen-containing cellulose ethers, some examples of which are described in U.S. Pat. No. 3,962,418. Other suitable polymers include synthetic polymers such as those disclosed in U.S. Publication No. 2007/0207109A1. Other suitable cationic polymers include copolymers of etherified cellulose, guar and starch, some examples of which are described in U.S. Pat. No. 3,958,581. When used, the cationic polymers herein are either soluble in the composition or are soluble in a complex coacervate phase in the composition formed by the cationic polymer and the anionic, amphoteric and/or zwitterionic surfactant component described hereinbefore. Complex coacervates of the cationic polymer can also be formed with other charged materials in the composition.

### E. Nonionic Polymers

The composition of the present invention may include a nonionic polymer. Polyalkylene glycols having a molecular weight of more than about 1000 are useful herein. Useful are those having the following general formula:

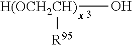

wherein R95 is selected from the group consisting of H, methyl, and mixtures thereof. Polyethylene glycol polymers useful herein are PEG-2M (also known as Polyox WSR® N-10, which is available from Union Carbide and as PEG-2,000); PEG-5M (also known as Polyox WSR® N-35 and Polyox WSR® N-80, available from Union Carbide and as PEG-5,000 and Polyethylene Glycol 300,000); PEG-7M (also known as Polyox WSR® N-750 available from Union Carbide); PEG-9M (also known as Polyox WSR® N-3333 available from Union Carbide); and PEG-14 M (also known as Polyox WSR® N-3000 available from Union Carbide),

### F. Conditioning Agents

Conditioning agents, and in particular silicones, may be included in the composition. Conditioning agents include any material which is used to give a particular conditioning benefit to hair and/or skin. In hair treatment compositions, suitable conditioning agents are those which deliver one or more benefits relating to shine, softness, combability, antistatic properties, wet-handling, damage, manageability, body, and greasiness. The conditioning agents useful in the compositions of the present invention typically comprise a water insoluble, water dispersible, non-volatile, liquid that forms emulsified, liquid particles. Suitable conditioning agents for use in the composition are those conditioning agents characterized generally as silicones (e.g., silicone oils, cationic silicones, silicone gums, high refractive silicones, and silicone resins), organic conditioning oils (e.g., hydrocarbon oils, polyolefins, and fatty esters) or combinations thereof, or those conditioning agents which otherwise form liquid, dispersed particles in the aqueous surfactant matrix herein. Such conditioning agents should be physically and chemically compatible with the essential components of the composition, and should not otherwise unduly impair product stability, aesthetics or performance.

The concentration of the conditioning agent in the composition should be sufficient to provide the desired conditioning benefits, and as will be apparent to one of ordinary skill in the art. Such concentration can vary with the conditioning agent, the conditioning performance desired, the average size of the conditioning agent particles, the type and concentration of other components, and other like factors.

### Silicones

The conditioning agent of the compositions of the present invention can be an insoluble silicone conditioning agent. The silicone conditioning agent particles may comprise volatile silicone, non-volatile silicone, or combinations thereof. Preferred are non-volatile silicone conditioning agents. If volatile silicones are present, it will typically be incidental to their use as a solvent or carrier for commercially available forms of non-volatile silicone materials ingredients, such as silicone gums and resins. The silicone conditioning agent particles may comprise a silicone fluid conditioning agent and may also comprise other ingredients, such as a silicone resin to improve silicone fluid deposition efficiency or enhance glossiness of the hair.

The concentration of the silicone conditioning agent typically ranges from about 0.01% to about 10%, preferably from about 0.1% to about 8%, more preferably from about 0.1% to about 5%, more preferably from about 0.2% to about 3%. Non-limiting examples of suitable silicone conditioning agents, and optional suspending agents for the silicone, are described in U.S. Reissue Pat. No. 34,584, U.S. Pat. No. 5,104,646, and U.S. Pat. No. 5,106,609. The silicone conditioning agents for use in the compositions of the present invention preferably have a viscosity, as measured at 25° C., from about 20 to about 2,000,000 centistokes (“cSt”), more preferably from about 1,000 to about 1,800,000 cSt, even more preferably from about 50,000 to about 1,500,000 cSt, more preferably from about 100,000 to about 1,500,000 cSt.

The dispersed silicone conditioning agent particles typically have a number average particle diameter ranging from about 0.01 μm to about 50 μm. For small particle application to hair, the number average particle diameters typically range from about 0.01 μm to about 4 μm, preferably from about 0.01 μm to about 2 μm, more preferably from about 0.01 μm to about 0.5 μm. For larger particle application to hair, the number average particle diameters typically range from about 4 μm to about 50 μm, preferably from about 6 μm to about 30 μm, more preferably from about 9 μm to about 20 μm, more preferably from about 12 μm to about 18 μm.

Background material on silicones including sections discussing silicone fluids, gums, and resins, as well as manufacture of silicones, are found in Encyclopedia of Polymer Science and Engineering, vol. 15, 2d ed., pp 204 308, John Wiley & Sons, Inc. (1989).

### Silicone Oils

Silicone fluids may include silicone oils, which are flowable silicone materials having a viscosity, as measured at 25° C., less than 1,000, cSt, preferably from about 5 cSt to about 1,000,000 cSt, more preferably from about 100 cSt to about 600,000 cSt. Suitable silicone oils for use in the compositions of the present invention include polyalkyl siloxanes, polyaryl siloxanes, polyalkylaryl siloxanes, polyether siloxane copolymers, and mixtures thereof. Other insoluble, non-volatile silicone fluids having hair conditioning properties may also be used.

### Amino and Cationic Silicones

Compositions of the present invention may include an aminosilicone. Aminosilicones, as provided herein, are silicones containing at least one primary amine, secondary amine, tertiary amine, or a quaternary ammonium group. Examples of aminosilicones include amodimethicone and terminal aminosilicones. Preferred aminosilicones may have less than about 0.5% nitrogen by weight of the aminosilicone, more preferably less than about 0.2%, more preferably still, less than about 0.1%. Higher levels of nitrogen (amine functional groups) in the amino silicone tend to result in less friction reduction, and consequently less conditioning benefit from the aminosilicone. It should be understood that in some product forms, higher levels of nitrogen are acceptable in accordance with the present invention.

Preferably, the aminosilicones used in the present invention have a particle size of less than about 50 μm once incorporated into the final composition. The particle size measurement is taken from dispersed droplets in the final composition. Particle size may be measured by means of a laser light scattering technique, using a Horiba model LA-910 Laser Scattering Particle Size Distribution Analyzer (Horiba Instruments, Inc.).

In one of the preferred embodiments, the aminosilicone has a viscosity of from about 1,000 cSt (centistokes) to about 1,000,000 cSt, more preferably from about 10,000 cSt to about 700,000 cSt, more preferably from about 50,000 cSt to about 500,000 cSt, and still more preferably from about 100,000 cSt to about 400,000 cSt. The viscosity of aminosilicones discussed herein is measured at 25° C.

In another preferred embodiment, the aminosilicone has a viscosity of from about 1,000 cSt to about 100,000 cSt, more preferably from about 2,000 cSt to about 50,000 cSt, more preferably from about 4,000 cSt to about 40,000 cSt, and still more preferably from about 6,000 cSt to about 30,000 cSt.

The aminosilicone is contained in the composition of the present invention at a level by weight of from about 0.05% to about 20%, preferably from about 0.1% to about 10%, and more preferably from about 0.3% to about 5%.

### Silicone Gums

Other silicone fluids suitable for use in the compositions of the present invention are the insoluble silicone gums. These gums are polyorganosiloxane materials having a viscosity, as measured at 25*C. of greater than or equal to 1,000,000 cSt. Silicone gums are described in U.S. Pat. No. 4,152,416; Noll and Walter, Chemistry and Technology of Silicones, New York: Academic Press (1968); and in General Electric Silicone Rubber Product Data Sheets SE 30, SE 33, SE 54 and SE 76. Specific non-limiting examples of silicone gums for use in the compositions of the present invention include polydimethylsiloxane, (polydimethylsiloxane) (methylvinyl-siloxane) copolymer, poly(dimethylsiloxane) (diphenyl siloxane)(methylvinylsiloxane) copolymer and mixtures thereof.

### High Refractive Index Silicones

Other non-volatile, insoluble silicone fluid conditioning agents that are suitable for use in the compositions of the present invention are those known as “high refractive index silicones,” having a refractive index of at least about 1.46, preferably at least about 1.48, more preferably at least about 1.52, more preferably at least about 1.55. The refractive index of the polysiloxane fluid will generally be less than about 1.70, typically less than about 1.60. In this context, polysiloxane “fluid” includes oils as well as gums.

The high refractive index polysiloxane fluid may include cyclic polysiloxanes such as those represented as:

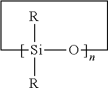

wherein R is as defined above, and n is a number from about 3 to about 7, preferably from about 3 to about 5.

Silicone fluids suitable for use in the compositions of the present invention are disclosed in U.S. Pat. No. 2,826,551, U.S. Pat. No. 3,964,500, U.S. Pat. No. 4,364,837, British Pat. No. 849,433, and Silicon Compounds. Petrarch Systems, Inc. (1984).

### Silicone Resins

Silicone resins may be included in the conditioning agent of the compositions of the present invention. These resins are highly cross-linked polymeric siloxane systems. The cross-linking is introduced through the incorporation of trifunctional and tetrafunctional silanes with monofunctional or difunctional, or both, silanes during manufacture of the silicone resin.

Silicone materials and silicone resins in particular, can conveniently be identified according to a shorthand nomenclature system known to those of ordinary skill in the art as “MDTQ” nomenclature. Under this system, the silicone is described according to presence of various siloxane monomer units which make up the silicone. Briefly, the symbol M denotes the monofunctional unit (CH3)3SiO0.5; D denotes the difunctional unit (CH3)2SiO; T denotes the trifunctional unit (CH3)SiO1.5; and Q denotes the quadra or tetra functional unit SiO2. Primes of the unit symbols (e.g. M′, D′, T′, and Q′) denote substituents other than methyl, and must be specifically defined for each occurrence.

Preferred silicone resins for use in the compositions of the present invention include, but are not limited to MQ, MT, MTQ, MDT and MDTQ resins. Methyl is a preferred silicone substituent. Especially preferred silicone resins are MQ resins, wherein the M:Q ratio is from about 0.5:1.0 to about 1.5:1.0 and the average molecular weight of the silicone resin is from about 1000 to about 10,000.

### Modified Silicones or Silicone Copolymers

Other modified silicones or silicone copolymers are also useful herein. Examples of these include silicone-based quaternary ammonium compounds (Kennan gnats) disclosed in U.S. Pat. Nos. 6,607,717 and 6,482,969; end-terminal quaternary siloxanes disclosed in German Patent No. DE 10036533; silicone aminopolyalkyleneoxide block copolymers disclosed in U.S. Pat. Nos. 5,807,956 and 5,981,681; hydrophilic silicone emulsions disclosed in U.S. Pat. No. 6,207,782; and polymers made up of one or more crosslinked rake or comb silicone copolymer segments disclosed in U.S. Pat. No. 7,465,439. Additional modified silicones or silicone copolymers useful herein are described in US Patent Application Nos. 2007/0286837A1 and 2005/0048549A1.

In alternative embodiments of the present invention, the above-noted silicone-based quaternary ammonium compounds may be combined with the silicone polymers described in U.S. Pat. Nos. 7,041,767 and 7,217,777 and US Publication No. 2007/0041929A1.

### Organic Conditioning Oils

The compositions of the present invention may also comprise from about 0.05% to about 3%, preferably from about 0.08% to about 1.5%, more preferably from about 0.1% to about 1%, of at least one organic conditioning oil as the conditioning agent, either alone or in combination with other conditioning agents, such as the silicones (described herein). Suitable conditioning oils include hydrocarbon oils, polyolefins, and fatty esters. Suitable hydrocarbon oils include include, but are not limited to, hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers and mixtures thereof. Straight chain hydrocarbon oils preferably are from about C12 to about C19. Branched chain hydrocarbon oils, including hydrocarbon polymers, typically will contain more than 19 carbon atoms. Suitable polyolefins include liquid polyolefins, more preferably liquid poly-□-olefins, more preferably hydrogenated liquid poly-□-olefins. Polyolefins for use herein are prepared by polymerization of C4 to about C14 olefenic monomers, preferably from about C6 to about C12. Suitable fatty esters include, but are not limited to, fatty esters having at least 10 carbon atoms. These fatty esters include esters with hydrocarbyl chains derived from fatty acids or alcohols (e.g. mono-esters, polyhydric alcohol esters, and di- and tri-carboxylic acid esters). The hydrocarbyl radicals of the fatty esters hereof may include or have covalently bonded thereto other compatible functionalities, such as amides and alkoxy moieties (e.g., ethoxy or ether linkages, etc.).

### Other Conditioning Agents

Also suitable for use in the compositions herein are the conditioning agents described by The Procter & Gamble Company in U.S. Pat. Nos. 5,674,478, and 5,750,122. Also suitable for use herein are those conditioning agents described in U.S. Pat. Nos. 4,529,586 (Clairol), 4,507,280 (Clairol), 4,663,158 (Clairol), 4,197,865 (L'Oreal), 4,217,914 (L'Oreal), 4,381,919 (L'Oreal), and 4,422,853 (L'Oreal).

### Anti-Dandruff Actives

The compositions of the present invention may also contain an anti-dandruff agent. Suitable, non-limiting examples of anti-dandruff actives include: antimicrobial actives, pyridinethione salts, azoles, selenium sulfide, particulate sulfur, keratolytic acid, salicylic acid, octopirox (piroctone olamine), coal tar, and combinations thereof. Preferred are pyridinethione salts. Such anti-dandruff particulate should be physically and chemically compatible with the essential components of the composition, and should not otherwise unduly impair product stability, aesthetics or performance.

Pyridinethione anti-dandruff agents are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982. It is contemplated that when ZPT is used as the anti-dandruff particulate in the compositions herein, that the growth or re-growth of hair may be stimulated or regulated, or both, or that hair loss may be reduced or inhibited, or that hair may appear thicker or

### Humectant

The compositions of the present invention may contain a humectant. The humectants herein are selected from the group consisting of polyhydric alcohols, water soluble alkoxylated nonionic polymers, and mixtures thereof. The humectants, when used herein, are preferably used at levels of from about 0.1% to about 20%, more preferably from about 0.5% to about 5%.

### Suspending Agent

The compositions of the present invention may comprise a suspending agent at concentrations effective for suspending water-insoluble material in dispersed form in the compositions or for modifying the viscosity of the composition. Such concentrations range from about 0.1% to about 10%, preferably from about 0.3% to about 5.0%.

Suspending agents useful herein include anionic polymers and nonionic polymers. Useful herein are vinyl polymers such as cross linked acrylic acid polymers with the CTFA name Carbomer, cellulose derivatives and modified cellulose polymers such as methyl cellulose, ethyl cellulose, hydroxyethyl cellulose, hydroxypropyl methyl cellulose, nitro cellulose, sodium cellulose sulfate, sodium carboxymethyl cellulose, crystalline cellulose, cellulose powder, polyvinylpyrrolidone, polyvinyl alcohol, guar gum, hydroxypropyl guar gum, xanthan gum, arabia gum, tragacanth, galactan, carob gum, guar gum, karaya gum, carragheenin, pectin, agar, quince seed (Cyclonia oblonga Mill), starch (rice, corn, potato, wheat), algae colloids (algae extract), microbiological polymers such as dextran, succinoglucan, pulleran, starch-based polymers such as carboxymethyl starch, methylhydroxypropyl starch, alginic acid-based polymers such as sodium alginate, alginic acid propylene glycol esters, acrylate polymers such as sodium polyacrylate, polyethylacrylate, polyacrylamide, polyethyleneimine, and inorganic water soluble material such as bentonite, aluminum magnesium silicate, laponite, hectonite, and anhydrous silicic acid.

Commercially available viscosity modifiers highly useful herein include Carbomers with tradenames Carbopol 934, Carbopol 940, Carbopol 950, Carbopol 980, and Carbopol 981, all available from B.F. Goodrich Company, acrylates/steareth-20 methacrylate copolymer with tradename ACRYSOL 22 available from Rohm and Hass, nonoxynyl hydroxyethylcellulose with tradename AMERCELL POLYMER HM-1500 available from Amerchol, methylcellulose with tradename BENECEL, hydroxyethyl cellulose with tradename NATROSOL, hydroxypropyl cellulose with tradename KLUCEL, cetyl hydroxyethyl cellulose with tradename POLYSURF 67, all supplied by Hercules, ethylene oxide and/or propylene oxide based polymers with tradenames CARBOWAX PEGs, POLYOX WASRs, and UCON FLUIDS, all supplied b Amerchol.

Other optional suspending agents include crystalline suspending agents which can be categorized as acyl derivatives, long chain amine oxides, and mixtures thereof. These suspending agents are described in U.S. Pat. No. 4,741,855. These preferred suspending agents include ethylene glycol esters of fatty acids preferably having from about 16 to about 22 carbon atoms. More preferred are the ethylene glycol stearates, both mono and distearate, but particularly the distearate containing less than about 7% of the mono stearate. Other suitable suspending agents include alkanol amides of fatty acids, preferably having from about 16 to about 22 carbon atoms, more preferably about 16 to 18 carbon atoms, preferred examples of which include stearic monoethanolamide, stearic diethanolamide, stearic monoisopropanolamide and stearic monoethanolamide stearate. Other long chain acyl derivatives include long chain esters of long chain fatty acids (e.g., stearyl stearate, cetyl palmitate, etc.); long chain esters of long chain alkanol amides (e.g., stearamide diethanolamide distearate, stearamide monoethanolamide stearate); and glyceryl esters (e.g., glyceryl distearate, trihydroxystearin, tribehenin) a commercial example of which is Thixin R available from Rheox, Inc. Long chain acyl derivatives, ethylene glycol esters of long chain carboxylic acids, long chain amine oxides, and alkanol amides of long chain carboxylic acids in addition to the preferred materials listed above may be used as suspending agents.

Other long chain acyl derivatives suitable for use as suspending agents include N,N-dihydrocarbyl amido benzoic acid and soluble salts thereof (e.g., Na, K), particularly N,N-di(hydrogenated) C.sub.16, C.sub.18 and tallow amido benzoic acid species of this family, which are commercially available from Stepan Company (Northfield, Ill., USA).

Examples of suitable long chain amine oxides for use as suspending agents include alkyl dimethyl amine oxides, e.g., stearyl dimethyl amine oxide.

Other suitable suspending agents include primary amines having a fatty alkyl moiety having at least about 16 carbon atoms, examples of which include palmitamine or stearamine, and secondary amines having two fatty alkyl moieties each having at least about 12 carbon atoms, examples of which include dipalmitoylamine or di(hydrogenated tallow)amine. Still other suitable suspending agents include (*hydrogenated tallow)phthalic acid amide, and crosslinked maleic anhydride-methyl vinyl ether copolymer.

### Aqueous Carrier

The compositions of the present invention can be in the form of pourable liquids (under ambient conditions). Such compositions will therefore typically comprise an aqueous carrier, which is present at a level of from about 20% to about 95%, more preferably from about 60% to about 85%. The aqueous carrier may comprise water, or a miscible mixture of water and organic solvent, but preferably comprises water with minimal or no significant concentrations of organic solvent, except as otherwise incidentally incorporated into the composition as minor ingredients of other essential or optional components.

The carrier useful in the present invention includes water and water solutions of lower alkyl alcohols and polyhydric alcohols. The lower alkyl alcohols useful herein are monohydric alcohols having 1 to 6 carbons, more preferably ethanol and isopropanol. The polyhydric alcohols useful herein include propylene glycol, hexylene glycol, glycerin, and propane diol.

### Dispersed Particles

The compositions may optionally comprise particles. The particles may be dispersed water-insoluble particles. The particles may be inorganic, synthetic, or semi-synthetic. In one embodiment, the particles have an average mean particle size of less than about 300 μm.

### Gel Matrix

A gel matrix is suitable for providing various conditioning benefits such as slippery feel during the application to wet hair and softness and moisturized feel on dry hair. In view of providing the above gel matrix, the cationic surfactant and the high melting point fatty compound are contained at a level such that the weight ratio of the cationic surfactant to the high melting point fatty compound is in the range of, preferably from about 1:1 to about 1:10, more preferably from about 1:1 to about 1:6.

### Hair Fixing Polymers

The compositions may optionally comprise hair fixing polymers. Hair fixing polymers may be selected from:
polymers with anionic or anionizable groups, selected from among terpolymers from acrylic acid, ethyl acrylate, and N-tert-butylacrylamide; crosslinked or uncrosslinked vinyl acetate/crotonic acid copolymers; terpolymers from tert-butylacrylate, ethyl acrylate and methacrylic acid; sodium polystyrenesulfonate; copolymers from vinyl acetate, crotonic acid and vinyl propionate; copolymers from vinyl acetate, crotonic acid and vinyl neodecanoate; aminomethylpropanol/acrylate copolymers; copolymers from vinylpyrrolidone and at least one further monomer selected from among acrylic acid, methacrylic acid, acrylic acid esters and methacrylic acid esters; copolymers from methyl vinyl ether and maleic acid monoalkyl esters; aminomethylpropanol salts of copolymers from allyl methacrylate and at least one further monomer selected from among acrylic acid, methacrylic acid, acrylic acid esters and methacrylic acid esters; crosslinked copolymers from ethyl acrylate and methacrylic acid; copolymers from vinyl acetate, mono-n-butyl maleate and isobornyl acrylate; copolymers from two or more monomers selected from among acrylic acid, methacrylic acid, acrylic acid esters and methacrylic acid esters, copolymers from octylacrylamide and at least one monomer selected from among acrylic acid, methacrylic acid, acrylic acid esters and methacrylic acid esters; polyesters from diglycol, cyclohexanedimethanol, isophthalic acid and sulfoisophthalic acid;
polymers with cationic or cationizable groups, selected from among cationic cellulose derivatives from hydroxyethylcellulose and diallyldimethylammonium chloride; cationic cellulose derivatives from hydroxyethylcellulose and with trimethylammonium substituted epoxides: poly(dimethyldiallylammonium chloride): copolymers from acrylamide and dimethyldiallylammonium chloride; quaternary ammonium polymers, formed from the reaction of diethyl sulfate with a copolymer from vinylpyrrolidone and dimethylaminoethyl methacrylate; quaternary ammonium polymers from methylvinylimidazolium chloride and vinylpyrrolidone; Polyquaternium-35; polymers from trimethylammoniumethyl methacrylate chloride; Polyquaternium-57; dimethylpolysiloxanes substituted with quaternary ammonium groups at the terminal positions; copolymers from vinylpyrrolidone, dimethylaminopropyl methacrylamide and methacryloylaminopropyllauryldimethylammonium chloride; chitosan and its salts; hydroxyalkyl chitosans and their salts; alkylhydroxyalkylchitosans and their salts; N-hydroxyalkylchitosan alkyl ethers; N-hydroxyalkylchitosan benzyl ethers; copolymers from vinylcaprolactam, vinylpyrrolidone and dimethylaminoethyl methacrylate; copolymers from vinylpyrrolidone and dimethylaminoethyl methacrylate, copolymers from vinylpyrrolidone, vinylcaprolactam and dimethylaminopropyl acrylamide; poly- or oligoesters formed from at least one first type of monomer that is selected from among hydroxyacids that are substituted with at least one quaternary ammonium group; terpolymers from vinylpyrrolidone, methacrylamide and vinylimidazole;
zwitterionic and/or amphoteric polymers, selected from among copolymers from octyl acrylamide, acrylic acid, butylaminoethyl methacrylate, methyl methacrylate and hydroxypropyl methacrylate; copolymers from lauryl acrylate, stearyl acrylate, ethylamine oxide methacrylate and at least one monomer selected from among acrylic acid, methacrylic acid, acrylic acid esters and methacrylic acid esters; copolymers from methacryloyl ethyl betaine and at least one monomer selected from among methacrylic acid and methacrylic acid esters; copolymer from acrylic acid, methyl acrylate and methacrylamidopropylfrimethylammonium chloride; oligomers or polymers that can be prepared from quaternary crotonoylbetaines or quaternary crotonoylbetaine esters;
nonionic polymers, selected from among polyvinylpyrrolidone, polyvinylcaprolactam, vinylpyrrolidone/vinyl acetate copolymers, polyvinyl alcohol, isobutylene/ethyl maleimide/hydroxyethyl maleimide copolymer; copolymers from vinylpyrrolidone, vinyl acetate and vinylpropionate.

In an embodiment, preferred hair fixing polymers are in a quantity of from about 0.01% to about 20% by total weight of the composition, more preferably from about 1% to about 10%.

### Skin Care Actives

The composition may comprise at least one skin care active, useful for regulating and/or improving the condition and/or appearance of mammalian skin. The skin care active may be soluble in oil or water, and may be present primarily in the oil phase and/or in the aqueous phase. Suitable actives include, but are not limited to, vitamins, peptides, sugar amines, sunscreens, oil control agents, tanning actives, anti-acne actives, desquamation actives, anti-cellulite actives, chelating agents, skin lightening agents, flavonoids, protease inhibitors, non-vitamin antioxidants and radical scavengers, hair growth regulators, anti-wrinkle actives, anti-atrophy actives, minerals, phytosterols and/or plant hormones, tyrosinase inhibitors, anti-inflammatory agents, N-acyl amino acid compounds, antimicrobials, and antifungals.

The composition may comprise from about 0.001% to about 10%, alternatively from about 0.01% to about 5%, of at least one vitamin. Herein, “vitamins” means vitamins, pro-vitamins, and their salts, isomers and derivatives. Non-limiting examples of suitable vitamins include: vitamin B compounds (including B1 compounds, B2 compounds, B3 compounds such as niacinamide, niacinnicotinic acid, tocopheryl nicotinate, C₁-C₁₈ nicotinic acid esters, and nicotinyl alcohol; B5 compounds, such as panthenol or “pro-B5”, pantothenic acid, pantothenyl; B6 compounds, such as pyroxidine, pyridoxal, pyridoxamine; carnitine, thiamine, riboflavin); vitamin A compounds, and all natural and/or synthetic analogs of Vitamin A, including retinoids, retinol, retinyl acetate, retinyl palmitate, retinoic acid, retinaldehyde, retinyl propionate, carotenoids (pro-vitamin A), and other compounds which possess the biological activity of Vitamin A; vitamin D compounds; vitamin K compounds; vitamin E compounds, or tocopherol, including tocopherol sorbate, tocopherol acetate, other esters of tocopherol and tocopheryl compounds; vitamin C compounds, including ascorbate, ascorbyl esters of fatty acids, and ascorbic acid derivatives, for example, ascorbyl phosphates such as magnesium ascorbyl phosphate and sodium ascorbyl phosphate, ascorbyl glucoside, and ascorbyl sorbate; and vitamin F compounds, such as saturated and/or unsaturated fatty acids. In one embodiment, the composition comprises a vitamin selected from the group consisting of vitamin B compounds, vitamin C compounds, vitamin E compounds and mixtures thereof. Alternatively, the vitamin is selected from the group consisting of niacinamide, tocopheryl nicotinate, pyroxidine, panthenol, vitamin E, vitamin E acetate, ascorbyl phosphates, ascorbyl glucoside, and mixtures thereof.

The composition may comprise one or more peptides. Herein, “peptide” refers to peptides containing ten or fewer amino acids, their derivatives, isomers, and complexes with other species such as metal ions (for example, copper, zinc, manganese, and magnesium). As used herein, peptide refers to both naturally occurring and synthesized peptides. In one embodiment, the peptides are di-, tri-, tetra-, penta-, and hexa-peptides, their salts, isomers, derivatives, and mixtures thereof. Examples of useful peptide derivatives include, but are not limited to, peptides derived from soy proteins, carnosine (beta-alanine-histidine), palmitoyl-lysine-threonine (pal-KT) and palmitoyl-lysine-threonine-threonine-lysine-serine (pal-KTTKS, available in a composition known as MATRIXYL®), palmitoyl-glycine-glutamine-proline-arginine (pal-GQPR, available in a composition known as RIGIN®), these three being available from Sederma, France, acetyl-glutamate-glutamate-methionine-glutamine-arginine-arginine (Ac-EEMQRR; Argireline®), and Cu-histidine-glycine-glycine (Cu-HGG, also known as IAMIN®). The compositions may comprise from about 1×10-7% to about 20%, alternatively from about 1×10-6% to about 10%, and alternatively from about 1×10-5% to about 5% of the peptide.

The composition may comprise a sugar amine, also known as amino sugars, and their salts, isomers, tautomers and derivatives. Sugar amines can be synthetic or natural in origin and can be used as pure compounds or as mixtures of compounds (e.g., extracts from natural sources or mixtures of synthetic materials). For example, glucosamine is generally found in many shellfish and can also be derived from fungal sources. Examples of sugar amines include glucosamine, N-acetyl glucosamine, mannosaminc. N-acetyl mannosamine, galactosamine. N-acetyl galactosamine, their isomers (e.g., stereoisomers), and their salts (e.g., HCl salt). Other sugar amine compounds useful in skin care compositions include those described in PCT Publication WO 02/076423 and U.S. Pat. No. 6,159,485, issued to Yu, et al. In one embodiment, the composition comprises from about 0.01% to about 15%, alternatively from about 0.1% to about 10%, and alternatively from about 0.5% to about 5%, of the sugar amine.

The composition may comprise one or more sunscreen actives (or sunscreen agents) and/or ultraviolet light absorbers. Herein, suitable sunscreen actives include oil-soluble sunscreens, insoluble sunscreens, and water-soluble sunscreens. In certain embodiments, the composition may comprise from about 1% to about 20%, or, alternatively, from about 2% to about 10%, by weight of the composition, of the sunscreen active and/or ultraviolet light absorber. Exact amounts will vary depending upon the chosen sunscreen active and/or ultraviolet light absorber and the desired Sun Protection Factor (SPF), and are within the knowledge and judgment of one of skill in the art.

Non-limiting examples of suitable oil-soluble sunscreens include benzophenone-3, bis-ethylhexyloxyphenol methoxyphenyl triazine, butyl methoxydibenzoyl-methane, diethylamino hydroxy-benzoyl hexyl benzoate, drometrizole trisiloxane, ethylhexyl methoxy-cinnamate, ethylhexyl salicylate, ethylhexyl triazone, octocrylene, homosalate, polysilicone-15, and derivatives and mixtures thereof.

Non-limiting examples of suitable insoluble sunscreens include methylene bis-benzotriazolyl tetramethylbutyl-phenol, titanium dioxide, zinc cerium oxide, zinc oxide, and derivatives and mixtures thereof.

Non-limiting examples of suitable water-soluble sunscreens include phenylbenzimidazole sulfonic acid (PBSA), terephthalylidene dicamphor sulfonic acid, (Mexoryl™ SX), benzophenone-4, benzophenone-5, benzylidene camphor sulfonic acid, cinnamidopropyl-trimonium chloride, methoxycinnamido-propyl ethyldimonium chloride ether, disodium bisethylphenyl triaminotriazine stilbenedisulfonate, disodium distyryibiphenyl disulfonate, disodium phenyl dibenzimidazole tetrasulfonate, methoxycinnamido-propyl hydroxysultaine, methoxycinnamido-propyl laurdimonium tosylate, PEG-25 PABA (p-aminobenzoic acid), polyquaternium-59. TEA-salicylate, and salts, derivatives and mixtures thereof.

Further examples of suitable sunscreens are disclosed as “Sunscreen Agents” in the Personal Care Product Council's International Cosmetic Ingredient Dictionary and Handbook, 13th Ed.,

The composition may comprise one or more compounds for regulating the production of skin oil, or sebum, and for improving the appearance of oily skin. Examples of suitable oil control agents include salicylic acid, dehydroacetic acid, benzoyl peroxide, vitamin B3 compounds (for example, niacinamide or tocopheryl nicotinate), their isomers, esters, salts and derivatives, and mixtures thereof. The compositions may comprise from about 0.0001% to about 15%, alternatively from about 0.01% to about 10%, alternatively from about 0.1% to about 5%, and alternatively from about 0.2% to about 2%, of an oil control agent.

The composition may comprise a tanning active. The compositions may comprise from about 0.1% to about 20%, from about 2% to about 7%, or, alternatively, from about 3% to about 6%, by weight of the composition, of a tanning active. A suitable tanning active includes dihydroxyacetone, which is also known as DHA or 1,3-dihydroxy-2-propanone.

The composition may comprise a safe and effective amount of one or more anti-acne actives. Examples of useful anti-acne actives include resorcinol, sulfur, salicylic acid, erythromycin, zinc, and benzoyl peroxide. Suitable anti-acne actives are described in further detail in U.S. Pat. No. 5,607,980. Further examples of suitable anti-acne actives are disclosed as “Antiacne Agents” in the Personal Care Product Council's International Cosmetic Ingredient Dictionary and Handbook, 13th Ed.

The composition may comprise a safe and effective amount of a desquamation active such as from about 0.01% to about 10%, from about 0.5% to about 5%, or, alternatively, from about 0.1% to about 2%, by weight of the composition. For example, the desquamation actives tend to improve the texture of the skin (e.g., smoothness). A suitable desquamation system comprises sulfhydryl compounds and zwitterionic surfactants and is described in U.S. Pat. No. 5,681,852. Another suitable desquamation system comprises salicylic acid and zwitterionic surfactants and is described in U.S. Pat. No. 5,652,228.

The composition may comprise a safe and effective amount of an anti-cellulite a ent. Suitable agents may include, but are not limited to, xanthine compounds (e.g., caffeine, theophylline, theobromine, and aminophylline).

Skin care compositions may comprise a safe and effective amount of a chelating agent such as from about 0.1% to about 10% or from about 1% to about 5% of the composition. Exemplary chelators are disclosed in U.S. Pat. No. 5,487,884; International Publication No. WO91/16035; and International Publication No. WO91/16034. A suitable chelator is furildioxime and derivatives.

The composition compositions may comprise a flavonoid. The flavonoid can be synthetic materials or obtained as extracts from natural sources, which also further may be derivatized. Examples of classes of suitable flavonoids are disclosed in U.S. Pat. No. 6,235,773.

The composition may comprise protease inhibitors including, but are not limited to, hexamidine compounds, vanillin acetate, menthyl anthranilate, soybean trypsin inhibitor, Bowman-Birk inhibitor, and mixtures thereof. Skin care compositions can include hexamidine compounds, its salts, and derivatives. As used herein, “hexaminide compound” means a compound having the formula:

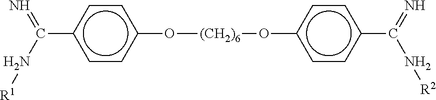

wherein R1 and R2 are optional or are organic acids (i.e., sulfonic acids, etc.). A particularly suitable hexamidine compound is hexamidine diisethionate.

The composition may other optional components such as non-vitamin antioxidants and radical scavengers, hair growth regulators, anti-wrinkle actives, anti-atrophy actives, minerals, phytosterols and/or plant hormones, tyrosinase inhibitors, anti-inflammatory agents, N-acyl amino acid compounds, antimicrobial or antifungal actives, and other useful skin care actives, which are described in further detail in U.S. application publication No. US 2006/0275237A1 and US 2004/0175347A1.

### Methods of Making Such Personal Care Compositions

### Hair Conditioner Formulations

Hair conditioners can be prepared by any conventional method well known in the art. They are suitably made as follows: deionized water is heated to 85° C. and cationic surfactants and high melting point fatty compounds are mixed in. If necessary, cationic surfactants and fatty alcohols can be pre-melted at 85° C. before addition to the water. The water is maintained at a temperature of about 85° C. until the components are homogenized, and no solids are observed. The mixture is then cooled to about 55° C. and maintained at this temperature, to form a gel matrix. Silicones, or a blend of silicones and a low viscosity fluid, or an aqueous dispersion of a silicone are added to the gel matrix. When included, poly alpha-olefin oils, polypropylene glycols, and/or polysorbates are also added to the gel matrix. When included, other additional components such as perfumes and preservatives are added with agitation. The gel matrix is maintained at about 50° C. durirw this time with constant stirring to assure homogenization. After it is homogenized, it is cooled to room temperature. A triblender and/or mill can be used in each step, if necessary to disperse the materials.

### Shampoo Compositions

Any suitable method of making the shampoo of the present invention may be used. In an embodiment, undecyl-based surfactant is blended with the other components of the shampoo compositions, according to standard methods known in the art. The typical procedure used for a clarifying shampoo would be to combine the undecyl sulfate paste or undeceth sulfate paste or mixtures thereof with water, add the desired water soluble co-surfactant and finish the composition by the addition preservatives, pH control agents, perfume, and salts to obtain the target physical properties. If a water insoluble co-surfactant is desired the surfactant and water mixture can be heated to a suitable temperature to facilitate its incorporation. If a rheology modifier is desired it can be added to the surfactant mixture prior the finishing step.

In the case of conditioning shampoos, typically the surfactant paste is combined with the co-surfactant as above and diluted with water to a target level commensurate to achieving the final activity. Rheology modifiers can be added at this point followed by conditioning agents, e.g. sucrose polyesters, silicones or silicone emulsions or other oils, cationic polymers from polymer premixes, perfumes, pearlizing agents or opacifiers, perfumes, and preservatives. Appropriate mixing steps to insure homogeneity are used as needed. The product is finished by the addition of pH control agents, hydrotropes, and salts to the desired physical properties.

### Compact Formulations

The present invention can also be used in a compact hair care formulation. A compact formula is a formula which delivers the same benefit to the consumer at a lower usage level. Compact formulations and methods of making compact formulations are described in US Application Publication No 2009/0221463A1.

### Methods of Using Such Personal Care Products

The present invention includes a method of delivering personal care benefits to the hair or skin. The method can include the steps of: topically applying a personal care composition onto the hair and/or skin; and rinsing the composition from the hair and/or skin by rinsing with water.

### Examples

The invention is further illustrated by the following examples. The examples are provided for illustrative purposes only. They are not to be construed as limiting the scope or content of the invention in any way.

### Example 1

### Materials and Methods

This example describes materials and methods used in carrying out the examples described herein. Although particular methods are described, one of ordinary skill in the art will understand that other, similar methods also can be used. In general, standard laboratory practices were used, unless otherwise stipulated. For example, standard laboratory practices were used for: cloning; manipulation and sequencing of nucleic acids; purification and analysis of proteins; and other molecular biological and biochemical techniques. Such techniques are explained in detail in standard laboratory manuals, such as Sambrook et al., Molecular Cloning: A Laboratory Manual, 3rd ed., vol. 1-3. Cold Spring Harbor, N.Y. (2000), and Ausubel et al., Current Protocols in Molecular Biology, Greene Publ. Assoc. & Wiley-Intersciences (1989).

Polymerase Chain Reaction (PCR):

PCR was used to amplify the specified nucleic acid sequences from DNA to create many of the expression constructs described herein. The primers used for the PCR reactions described herein are listed in Table 7,

TABLE 7
Primers

| Name | Sequence 5′ to 3′ |
|---|---|
| fadD9F | cat ATGTCGATCAACGATCAGCGACTGAC |
| | (SEQ ID NO: 1) |
| fadD9R | cctagg TCACAGCAGCCCGAGCAGTC |
| | (SEQ ID NO: 2) |
| CARMCaF | cat ATGACGATCGAAACGCG |
| | (SEQ ID NO: 3) |
| CARMCaR | cctagg TTACAGCAATCCGAGCATCT |
| | (SEQ ID NO: 4) |
| CARMCbF | cat ATGACCAGCGATGTTCAC |
| | (SEQ ID NO: 5) |
| CARMCbR | cctagg TCAGATCAGACCGAACTCACG |
| | (SEQ ID NO: 6) |
| TesA-F | CATATGGCGGACACGTTATTGATT |
| | (SEQ ID NO: 111) |
| TesA-R | CTAGGTTATGAGTCATGATTTACTAAAG |
| | (SEQ ID NO: 112) |
| YjgBF | aatcc TGGCATCGATGATAAAAAGCTATGCCGCAAAAG |
| | (SEQ ID NO: 113) |
| YjgBR | ataaaagct TTCAAAAATCGGCTTTCAACACCACGCGG |
| | (SEQ ID NO: 114) |
| ADP1Alrmut | GATGAGCTCAAAGCTATGGGGGCCGATCACGTGGTC |
| 1F | (SEQ ID NO: 115) |
| ADP1Alrmut | GACCACGTGATCGGCCCCCATAGCTTTGAGCTCATC |
| 1R | (SEQ ID NO: 116) |
| ADP1Alr1F | AATACCATGGCAACAACTAATGTGATTCATGCTTATGCTGCA |
| | (SEQ ID NO: 117) |
| ADP1Alr1R | ATAAAAGCTTTTAAAAATCGGCTTTAAGTACAATCCGATAAC |
| | (SEQ ID NO: 118) |
| YafV_NotI | caaccaGCGGCCGCgcgacgaagctgccgcttc |
| Ivry_Ol | cctacaagtaaggggcttttcgttatgaataacggagccgaaaggctcc |
| Lpcaf_ol | ctttcggctccgttattcataacgaaaagccccttacttgtaggagg |
| LpcaR_Bam | ccaGGATCCaggtcggatgcggcgtgaac |
| fad1 | TAACCGGCGTCTGACGACTGACTTAACGCTCAGGCTTTATTGTCCAC |
| | TTTGTGTAGGCTGGAGCTGCTTCG |
| | (SEQ ID NO: 119) |
| fad2 | CATTTGGGGTTGCGATGACGACGAACACGCATTTTAGAGGTGAAGA |
| | ATTGCATATGAATATCCTCCTTTAGTTCC |
| | (SEQ ID NO: 120) |
| fadF | CGTCCGTGGTAATCATTTGG |
| | (SEQ ID NO: 121) |
| fadR | TCGCAACCTTTTCGTTGG |
| | (SEQ ID NO: 122) |
| yjgBRn | GCGCCTCAGATCAGCGCTGCGAATGATTTTCAAAAATCGGCTTTCAA |
| | CACTGTAGGCTGGAGCTGCTTCG |
| | (SEQ ID NO: 123) |
| yjgBFn | CTGCCATGCTCTACACTTCCCAAACAACACCAGAGAAGGACCAAAA |
| | AATGATTCCGGGGATCCGTCGACC |
| | (SEQ ID NO: 124) |
| BF | gtgctggcgataCGACAAAACA |
| | (SEQ ID NO: 125) |
| BR | CCCCGCCCTGCCATGCTCTACAC |
| | (SEQ ID NO: 126) |
| fadD-F | CATGCCATGGTGAAGAAGGTTTGGCTTAA |
| | (SEQ ID NO: 127) |
| fadD-R | CCCAAGCTTTCAGGCTTTATTGTCCAC |
| | (SEQ ID NO: 128) |

### Fatty Alcohol Detection Methods:

Detection Method 1: 20 min GC/MS method GC-MS was performed using an Agilent 6850 Series II GC system coupled with an Agilent 5975B VL MSD mass spectrometer. A 30 m×0.25 mm (0.10 μm film) DB-5 (5% phenyl methyl siloxane) column was installed. The spiltless inlet of GC was held at 300° C. The column was held isothermal at 100° C. for 3 min, then ramped from 100° C. to 320° C. at a rate of 20° C./min. When the final temperature was reached, the column remained isothermal for 5 min at 320° C. The injection volume was 1 μL. The carrier gas, helium, was released at 1.2 mL/min. The transfer line from the GC to the MS was held at 300° C. The mass spectrometer was equipped with an electron impact ionization (EI) source. The EI source temperature was set at 230° C. The mass spectrometer scan range was from 50 m/z to 550 m/z.

Detection Method 2: 15 m 6 min GC/MS method GC-MS was performed using an Agilent 6890N GC system coupled with an Agilent 5975B inert XL ET/C1 MSD system. A 15 m×0.25 mm (0.10 μm film) DB-1HT (100% dimethylpolysiloxane) column was installed. The spiltless inlet of GC was held at 300° C. The column was held isothermal at 120° C. for 0.3 min, then ramped from 120° C. to 320° C. at a rate of 40° C./min. When the final temperature was reached, the column remained isothermal for 0.2 min at 320° C. The injection volume was 1 μL. The carrier gas, helium, was released at 1.3 mL/min. The transfer line from the GC to the MS was held at 300° C. The mass spectrometer was equipped with an electron impact ionization (EI) source. The EI source temperature was set at 230° C. The mass spectrometer scan range was from 50 m/z to 550 m/z.

Detection Method 3: 15 m 9 min GC/MS method GC-MS was performed using an Agilent 6890N GC system coupled with an Agilent 5975B inert XL ET/CI MSD system. A 15 m×0.25 mm (0.10 μm film) DB-1HT (100% dimethylpolysiloxane) column was installed. The spiltless inlet of GC was held at 300° C. The column was held isothermal at 100° C. for 0.5 min, then ramped from 100° C. to 260° C. at a rate of 50° C./min. When the final temperature was reached, the column remained isothermal for 0.5 minutes at 260° C. The injection volume was 1 μL. The carrier gas, helium, was released at 1.3 mL/min. The transfer line from the GC to the MS was held at 300° C. The mass spectrometer was equipped with an electron impact ionization (EI) source. The EI source temperature was set at 230° C. The mass spectrometer scan range was from 50 m/z to 550 m/z.

### Example 2

### Identification of Carboxylic Acid Reductase (CAR) Homologs

The carboxylic acid reductase (CAR) from Nocardia sp. strain NRRL 5646 can reduce carboxylic acids (e.g., benzoate) into their corresponding aldehydes without utilizing separate activating enzymes, such as acyl-CoA synthases (Li et al., J. Bacterial., 179: 3482-3487 (1997); He et al., Appl. Environ. Microbial., 70: 1874-1881 (2004)).

A BLAST search using the NRRL 5646 CAR amino acid sequence (Genpept accession AAR91681) (SEQ ID NO:16) as the query sequence identified approximately 20 homologous sequences. Three homologs, listed in Table 8, were evaluated for their ability to convert fatty acids into fatty aldehydes in vivo when expressed in E. coli.

At the nucleotide sequence level, carA (SEQ ID NO:19), carB (SEQ ID NO:21), and fadD9 (SEQ ID NO:23) demonstrated 62.6%, 49.4%, and 60.5% homology, respectively, to the car gene (AY495697) of Nocardia sp. NRRL 5646 (SEQ ID NO:15). At the amino acid level, CARA (SEQ ID NO:20), CARB (SEQ ID NO:22), and FadD9 (SEQ ID NO:24) demonstrated 62.4%, 59.1% and 60.7% identity, respectively, to CAR of Nocardia sp. NRRL 5646 (SEQ ID NO:16).

TABLE 8
CAR-like Protein and the Corresponding Coding Sequences

| Genpept | | | |
|---|---|---|---|
| Accession | Locus_tag | Annotation in GenBank | Gene Name |
| NP_217106 | Rv 2590 | Probable fatty-acid-CoA ligase | fadD9 |
| | | (FadD9) | |
| ABK75684 | MSMEG | NAD dependent | carA |
| | 2956 | epimerase/dehydratase family | |
| | | protein | |
| YP_889972.1 | MSMEG | NAD dependent | carB |
| | 5739 | epimerase/dehydratase family | |
| | | protein | |

### Example 3

### Identification of Alcohol Dehydrogenase (AlrA) Homologs

AlrA is an alcohol dehydrogenase in Acinetobacter sp. M-1 involved in ester biosynthesis from a non-fatty alcohol carbon source (Tani et al., Appl. Environ. Microbiol. 66: 5231-5235 (2000)). A BLAST search of the genomic and protein databases of E. coli K12 and Acinetobacter baylyi ADP1 for homologs of AlrA identified YjgB (GenBank accession number, NP_{—}418690, 57% identical to AlrA) from E. coli strain K-12 and AlrAadp1 (GenPept accession number CAG70248.1, 79% identical to AlrA) from Acinetobacter baylyi ADP1. A BLAST search of the protein databases of E. coli K12 for homologs of YjgB further identified YahK (Genbank accession number NP_{—}414859, 35% identical to YjgB).

### Example 4

### Expression of CAR Homologs and Alcohol Dehydrogenase in E. coli

CAR Plasmid Construction

Three E. coli expression plasmids were constructed to express the genes encoding the CAR homologs listed in Table 8. First, fadD9 was amplified from genomic DNA of Mycobacterium tuberculosis H37Rv (obtained from The University of British Columbia, Vancouver, BC, Canada) using the primers fadD9F and FadD9R (see Table 7). The PCR product was first cloned into PCR-blunt (Invitrogen) and then released as an NdeI-AvrII fragment. The NdeI-AvrII fragment was then cloned between the NdeI and AvrII sites of pACYCDuet-1 (Novagen) to generate pACYCDuet-1-fadD9.

The carA gene was amplified from the genomic DNA of Mycobacterium smegmatis MC2 155 (obtained from ATCC (ATCC 23037D-5)) using primers CARMCaF and CARMCaR (see Table 7). The carB gene was amplified from the genomic DNA of Mycobacterium smegmatis MC2 155 (obtained from ATCC (ATCC 23037D-5)) using primers CARMCbF and CARMCbR (see Table 7). Each PCR product was first cloned into PCR-blunt and then released as an NdeI-AvrII fragment. Each of the two fragments was then subcloned between the NdeI and AvrII sites of pACYCDuet-1 (Novogen) to generate pACYCDuet-1-carA and pACYCDuet-1-carB.

Thioesterase Plasmid Construction

The ′tesA gene of E. coli (thioesterase A gene accession NP_{—}415027, EC 3.1.1.5, 3.1.2.-) without the leader sequence (Cho and Cronan, J. Biol. Chem., 270: 4216-69 (1995)) was amplified using the primers TesA-F and TesA-R (see Table 7). The PCR product was cloned into NdeI/AvrII digested pETDuet-1 (Novagen) to generate pETDuet-1-′tesA.

Alcohol Dehydrogenase Plasmid Construction

The plasmid pETDuet-1-′tesA-yjgB carries ′tesA and yjgB (a putative alcohol dehydrogenase; GenBank accession number, NP_{—}418690; GenPept accession number AAC77226) from the E. coli K12 strain.

The gene yjgB (GenBank accession number, NP_{—}418690) was amplified from the genomic DNA of E. coli K-12 using the primers YjgBF and YjgBR (see Table 7). The PCR product was then subcloned into the NcoI and HindIII sites of pETDuet-1-′tesA to generate pETDuet-1-′tesA-yjgB.

The plasmid pETDuet-1-′tesA-alrAadp1 carries ′tesA and alrAadp1 (GenPept accession number CAG70248.1) from Acinetobacter baylyi ADP1.

The gene alrAadp1 was amplified from the genomic DNA of Acinetobacter baylyi ADP1 by a two-step PCR procedure. The first set of PCR reactions eliminated an internal NcoI site at bp 632-636 using the primers ADP1Alrmut1F and ADPIAlrmut1R (see Table 7). The PCR products were then isolated, purified using the Qiagen gel extraction kit, and used as inputs for a second PCR reaction using the primers ADP1Alr1F and ADP1Alr1R (see Table 7) to produce full-length AlrAadp1 with a C→T mutation at position 633.

The plasmid pETDuet-1-′tesA-alrAadp1 was prepared by inserting the alrAadp1 gene (gene locus-tag=“ACIAD3612”) into the NcoI and HindIII sites of pETDuet-1-′tesA.

Evaluation of Fatty Aldehyde and Fatty Alcohol Production

In order to evaluate the affect of carboxylic acid reductases and alcohol dehydrogenases on the production of fatty alcohols, various combinations of the prepared plasmids were transformed in the E. coli strain C41 (DE3, ΔfadE), which was produced by modifying the E. coli strain C41(DE3) from, for example, Lucigen (Middleton, Wis.) or Overexpress.com (Saint Beausine, France) to knock-out the acyl-CoA dehydrogenase gene fadE. Briefly, primers YafV_NotI and Ivry_Ol (see Table 7) were used to amplify about 830 bp upstream of fadE and primers Lpcaf_ol and LpcaR_Bam (see Table 7) were used to amplify about 960 hp downstream of fadE. Overlap PCR was used to create a construct for in-frame deletion of the complete fadE gene. The fadE deletion construct was cloned into the temperature-sensitive plasmid pKOV3, which contained a sacB gene for counterselection. The chromosomal deletion of fadE was made according to the method of Link et al., J. Bact., 179: 6228-6237 (1997). The resulting fadE deletion strain is not capable of degrading fatty acids and fatty acyl-CoAs, and is herein designated as E. coli C41 (DE3, ΔfadE).

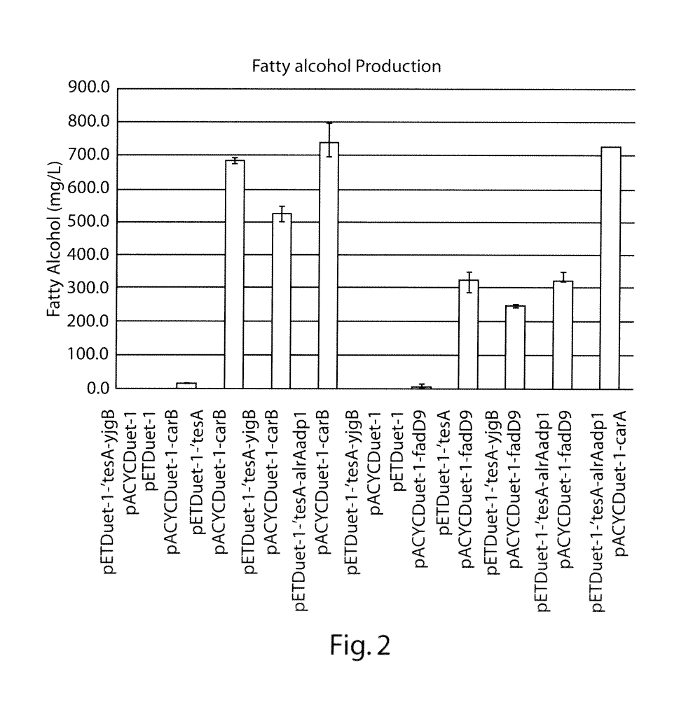

For example, the plasmid pACYCDuet-1-carA, encoding the CAR homolog carA, was co-transformed with pETDuet-1-′tesA-alrAadp1 (see, e.g., FIG. 2).

The plasmid pACYCDuet-1-carB, encoding the CAR homolog carB, was co-transformed with pETDuet-1-′tesA. In addition, pACYCDuet-1-carB was also separately co-transformed with pETDuet-1-′tesA-yjgB and pETDuet-1-′tesA-alrAadp1. As a control, pACYCDuet-1-carB was co-transformed with the empty vector pETDuet-1 (see, e.g., FIG. 2).

The plasmid pACYCDuet-1-fadD9, encoding the CAR homolog fadD9, was co-transformed with pETDuet-1-′tesA. In addition, pACYCDuet-1-fadD9 was also separately co-transformed with pETDuet-1-′tesA-yjgB and pETDuet-1-′tesA-alrAadp1. As a control, pACYCDuet-1-fadD9 was co-transformed with the empty vector pETDuet-1 (see, e.g., FIG. 2).

As an additional control, pETDuet-1-′tesA-yjgB was co-transformed with the empty vector pACYCDuet-1.

The E. coli transformants were grown in 3 mL of LB medium supplemented with carbenicillin (100 mg/L) and chloramphenicol (34 mg/L) at 37° C. After overnight growth, 15 μL of culture was transferred into 2 mL of fresh LB medium supplemented with carbenicillin and chloramphenicol. After 3.5 hours of growth, 2 mL of culture were transferred into a 125 mL flask containing 20 mL of M9 medium with 2% glucose and with carbenicillin and chloramphenicol. When the OD₆₀₀ of the culture reached 0.9, 1 mM of IPTG was added to each flask. After 20 hours of growth at 37° C., 20 mL of ethyl acetate (with 1% of acetic acid, v/v) was added to each flask to extract the fatty alcohols produced during the fermentation. The crude ethyl acetate extract was directly analyzed with GC/MS as described herein.

The measured retention times were 6.79 minutes for cis-5-dodecen-1-ol, 6.868 minutes for 1-dodecanol, 8.058 minutes for cis-7-tetradecen-1-ol, 8.19 minutes for 1-tetradecanol, 9.208 minutes for cis-9-hexadecen-1-ol, 9.30 minutes for 1-hexadecanol, and 10.209 minutes for cis-11-octadecen-1-ol.

The co-expression of the leaderless tesA (′tesA) and any of the three car genes in E. coli resulted in high titers of fatty alcohols and detectable fatty aldehyde production (FIGS. 2, 3, and 5). The expression of carA or carB with the leaderless tesA and alrAadp1 resulted in fatty alcohol titers of greater than 700 mg/L and reduced fatty aldehyde production. Likewise, fadD9 co-expressed with the leaderless tesA and alrAadp1 produced over 300 mg/L of fatty alcohol. When expressed without the leaderless tesA, neither carB nor fadD9 produced more than 10 mg/L of fatty alcohols (possibly resulting from the accumulation of free fatty acids in the cell due to endogenous tesA). Taken together, this data indicates that fatty acids are the substrates for these CAR homologs and that overexpression of a thioesterase, such as ′tesA (to release fatty acids from acyl-ACP), achieves significant production of fatty alcohols.

In one fermentation, E. coli strain C41 (DE3, ΔfadE) co-transformed with pACYCDuet-1-carB+pETDuet-1-′tesA produced an average of 695 mg/L of fatty alcohols and 120 mg/L of fatty aldehydes. The presence of large amounts of fatty aldehydes is consistent with CAR being an aldehyde-generating, fatty acid reductase (AFAR). This mechanism is different from alcohol-generating fatty acyl-CoA reductases (FAR), represented by JjFAR (GenPept accession number AAD38039), and fatty acyl-CoA reductases, represented by Acr1 (GenPept accession number CAG70047).

The production of fatty alcohols from fatty aldehydes in the E. coli strains described above may have been catalyzed by an endogenous alcohol dehydrogenase(s). E. coli produces an alcohol dehydrogenase(s) (e.g., yjgB) capable of converting fatty aldehydes of various chain-length into fatty alcohols (Naccarato et al., Lipids. 9: 419-428 (1974); Reiser et al., J. Bacterial., 179: 2969-2975 (1997)).

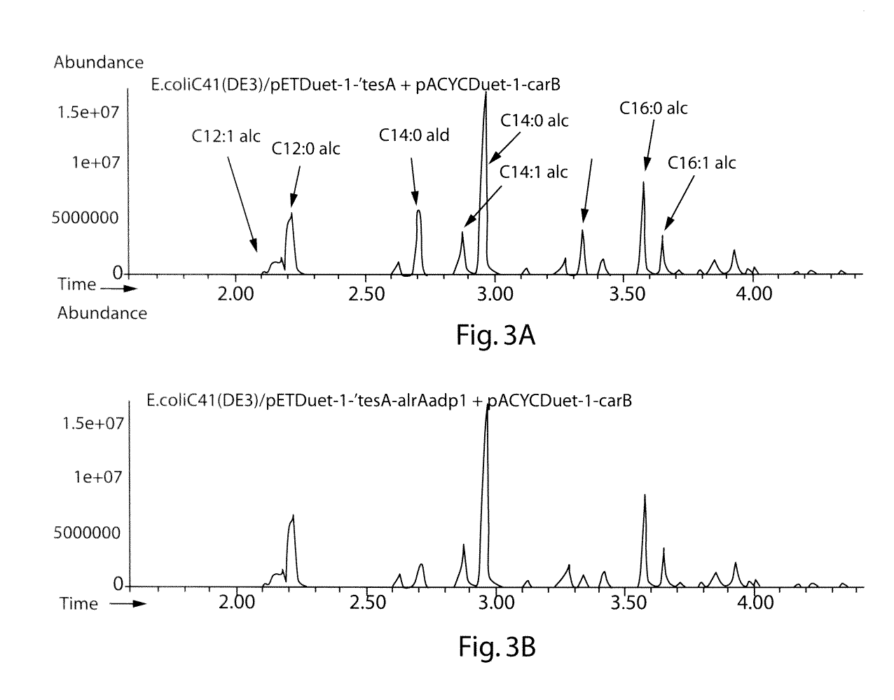

Therefore, alcohol dehydrogenases may also play a role in the fatty alcohol biosynthetic pathway in addition to carboxylic acid reductases. For example, expression of either yjgB or alrAadp1 with carB and the leaderless tesA significantly reduced the accumulation of fatty aldehyde, compared to strains that did not overexpress yjgB or alrAadp1 (FIGS. 3A and 3B).

Following the fermentations where pACYCDuet-1-carB was transformed in E. coli strain C41 (DE3, ΔfadE), a white, round, disk-like deposit was observed at the bottom center of the flasks used for fatty alcohol production with recombinant E. coli strains. In contrast, no such deposits were observed at the bottom of the control flasks which did not express car homologs. GC/MS analysis of the deposit dissolved in ethyl acetate (with of acetic acid, v/v) revealed that the deposit was a fatty alcohol deposit.

Types of Fatty Alcohols Produced by Different CAR Homologs

Depending upon the CAR homolog expressed in E. coli strain C41 (DE3, ΔfadE), different mixtures of fatty alcohols were produced. Different compositions of fatty alcohols were observed among the three CAR homologs evaluated (see Table 9). FadD9 produced more C₁₂ fatty alcohols relative to other fatty alcohols with carbon chain lengths greater than 12. Both CarA and CarB produced a wider range in chain length of fatty alcohols than was observed when expressing FadD9.

TABLE 9
Acyl-composition of Fatty Alcohols Produced by
Recombinant E. coli Strains

| Expressed with | Acyl-composition of Fatty Alcohols (%) | | | | | | |
|---|---|---|---|---|---|---|---|
| TesA* and AlrAadp1 | C10:0 | C12** | C14:1 | C14:0 | C16:1 | C16:0 | C18:1 |
| CarA | trace | 38 | 13 | 27 | 16 | 4 | 3 |
| FadD9 | trace | 63 | 14 | 16 | 7 | trace | trace |
| CarB | trace | 32 | 11 | 41 | 12 | trace | trace |

*leaderless TesA.
**C12, including C12:0 and C12:1 fatty alcohol.

Quantification and Identification of Fatty Alcohols

GC-MS was performed using Detection Method 1, as described in Example 1. Prior to quantification, various alcohols were identified using two methods. First, the GC retention time of each compound was compared to the retention time of known standards, such as cetyl alcohol, dodecanol, tetradecanol, octadecanol, and cis-9-octadecenol. Second, identification of each compound was confirmed by matching the compound's mass spectrum to a standard's mass spectrum in the mass spectra library (e.g., C12:0, C12:1, C13:0, C14:0, C14:1, C15:0, C16:0, C16:1, C17:0, C18:0 and C18:1 alcohols).

### Example 5

### Production of Fatty Alcohols by Heterologous Expression of CAR Homologs in E. coli MG1655 (DE3, ΔfadD)

Construction of fadD Deletion Strain

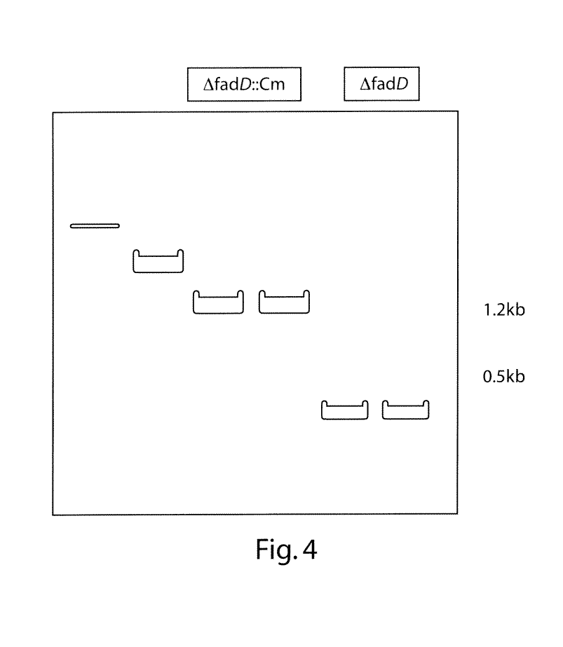

The fadD gene of E. coli MG1655 was deleted using the lambda red system (Datsenko et al., Proc. Natl. Acad. Sci. USA, 97: 6640-6645 (2000)). Briefly, the chloramphenicol acetyltransferase gene from pKD3 was amplified with the primers fad1 and fad2 (see Table 7). This PCR product was electroporated into E. coli MG1655 (pKD46). The cells were plated on L-chloramphenicol (30 μg/mL) (L-Cm) and grown overnight at 37° C. Individual colonies were picked on to another L-Cm plate and grown at 42° C. These colonies were then patched to L-Cm and L-carbenicillin (100 mg/mL) (L-Cb) plates and grown at 37° C. overnight. Colonies that were Cm^{R} and Cb^{S} were evaluated further by PCR to ensure the PCR product inserted at the correct site. PCR verification was performed on colony lysates of these bacteria using the primers fadF and fadR (see Table 7). Expected size of the ΔfadD::Cm deletion was about 1200 bp (FIG. 4). The chloramphenicol resistance gene was eliminated using a FLP helper plasmid as described in Datsenko et al., Proc. Natl. Acad. Sci. USA, 97: 6640-6645 (2000). PCR verification of the deletion was performed with primers fadF and fadR (see Table 7) (FIG. 4). The MG1655 ΔfadD strain was unable to grow on M9+oleate agar plates (oleate as carbon source). It also was unable to grow in M9+oleate liquid media. The growth defect was complemented by an E. coli fadD gene supplied in trans (in pCL1920-Ptrc).

Construction of MG1655(DE3, ΔfadD) Strain

To generate a T7-responsive strain, the λDE3 Lysogenization Kit (Novagen), which is designed for site-specific integration of λDE3 prophage into an E. coli host chromosome, was utilized such that the lysogenized host can be used to express target genes cloned in T7 expression vectors. λDE3 is a recombinant phage carrying the cloned gene for T7 RNA polymerase under lacUV5 control. Briefly, the host strain was cultured in LB supplemented with 0.2% maltose, 10 mM MgSO₄, and antibiotics at 37° C. to an OD₆₀₀ of 0.5. Next, 10⁸ pfu λDE3, 10⁸ pfu Helper Phage, and 10⁸ pfu Selection Phage were incubated with 10 μL host cells. The host/phage mixture was incubated at 37° C. for 20 min to allow phage to adsorb to host. Finally, the mixture was pipeted onto an LB plate supplemented with antibiotics. The mixture was spread evenly using plating beads, and the plates were inverted and incubated at 37° C. overnight. λDE3 lysogen candidates were evaluated by their ability to support the growth of the T7 Tester Phage. T7 Tester Phage is a T7 phage deletion mutant that is completely defective unless active T7 RNA polymerase is provided by the host cell. The T7 Tester Phage makes very large plaques on authentic λDE3 lysogens in the presence of IPTG, while much smaller plaques are observed in the absence of inducer. The relative size of the plaques in the absence of IPTG is an indication of the basal level expression of 17 RNA polymerase in the lysogen, and can vary widely between different host cell backgrounds.

The following procedure was used to determine the presence of DE3 lysogeny. First, candidate colonies were grown in LB supplemented with 0.2% maltose, 10 mM MgSO₄, and antibiotics at 37° C. to an OD₆₀) of 0.5. An aliquot of T7 Tester Phage was then diluted in 1× Phage Dilution Buffer to a titer of 2×10³ pfu/mL. In duplicate tubes, 100 μL host cells were mixed with 100 μL diluted phage. The host/phage mixture was incubated at room temperature for 10 min to allow phage to adsorb to host. Next, 3 mL of molten top agarose was added to each tube containing host and phage. The contents of one duplicate were plated onto an LB plate, and the contents of the other duplicate were plated onto an LB plate supplemented with 0.4 mM IPTG (isopropyl-b-thiogalactopyranoside) to evaluate induction of T7 RNA polymerase. Plates were allowed to sit undisturbed for 5 min until the top agarose hardened. The plates were then inverted at 30° C. overnight.

Construction of MG1655(DE3, ΔfadD, yjgB::kan) Strain

The yjgB knockout strain, MG1655(DE3, ΔfadD, yjgB::kan), was constructed using the lambda red system (Datsenko et al., Proc. Natl. Acad. Sci. USA, 97: 6640-6645 (2000)). Briefly, the kanamycin resistant gene from pKD13 was amplified with the primers yjgBRn and yjgBFn (see Table 7). The PCR product was then electroporated into E. coli MG1655(DE3, ΔfadD)/pKD46. The cells were plated on kanamycin (50 μg/mL) (LA-Kan) and grown overnight at 37° C. Individual colonies were picked on to another L-Kan plate and grown at 42° C. These colonies were then patched to LA-Kan and carbenicillin (100 mg/mL) (LA-Cb) plates and grown at 37° C. overnight. Colonies that were kan^{R} and Cb^{S} were evaluated further by PCR to ensure the PCR product was inserted at the correct site. PCR verification was performed on colony lysates of these bacteria using the primers BF and BR. The expected size of the yjgB::kan knockout was about 1450 bp.

Evaluation of FadD on Fatty Alcohol Production Using MG1655DE3 ΔFadD) Strain

In Example 2, a fadE deletion strain was used for fatty aldehyde and fatty alcohol production from ′tesA, CAR homologs, and endogenous alcohol dehydrogenase(s) in E. coli. To demonstrate that CAR homologs used fatty acids instead of acyl-CoA as a substrate, the gene encoding for acyl-CoA synthase in E. coli (fadD) was deleted so that the fatty acids produced were not activated. E. coli strain MG1655(DE3, ΔfadD) was transformed with pETDuet-1-′tesA and pACYCDuet-1-carB. The transformants were evaluated for fatty alcohol production using the methods described herein. These transformants produced about 360 mg/L of fatty alcohols (dodecanol, dodecenol, tetradecanol, tetradecenol, hexadecanol, hexadecenol, and octadecenol).

YjgB is an Alcohol Dehydrogenase

To confirm that YjgB was an alcohol dehydrogenase responsible for converting fatty aldehydes into their corresponding fatty alcohols, pETDuet-1-′tesA and pACYCDuet-1-fadD9 were co-transformed into either MG1655(DE3. ΔfadD) or MG1655(DE3, ΔfadD, yjgB::kan). At the same time, MG1655(DE3, ΔfadD, yjgB::kan) was transformed with both pETDuet-1-′tesA-yjgB and pACYCDuet-1-fadD9. The E. coli transformants were grown in 3 mL of LB medium supplemented with carbenicillin (100 mg/L) and chloramphenicol (34 mg/L) at 37° C. After overnight growth, 15 μL of culture was transferred into 2 mL of fresh LB medium supplemented with carbenicillin and chloramphenicol. After 3.5 hrs of growth, 2 mL of culture was transferred into a 125 mL flask containing 20 mL of M9 medium with 2% glucose, carbenicillin, and chloramphenicol. When the OD₆₀₀ of the culture reached 0.9, 1 mM of IPTG was added to each flask. After 20 h of growth at 37° C., 20 mL of ethyl acetate (with 1% of acetic acid, v/v) was added to each flask to extract the fatty alcohols produced during the fermentation. The crude ethyl acetate extract was directly analyzed with GC/MS as described herein.

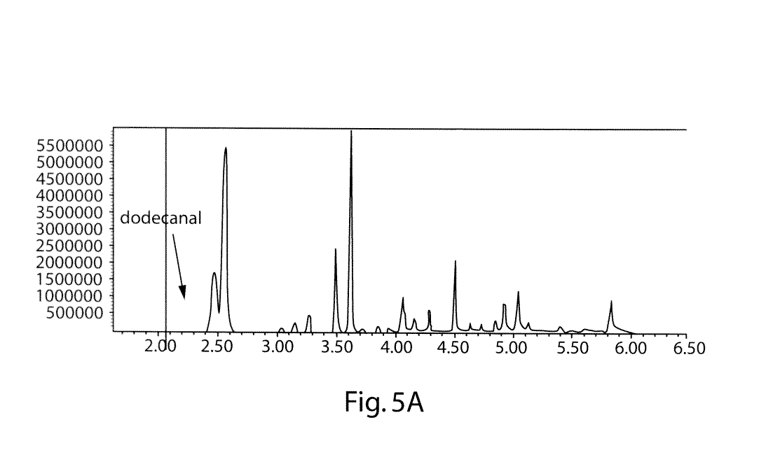

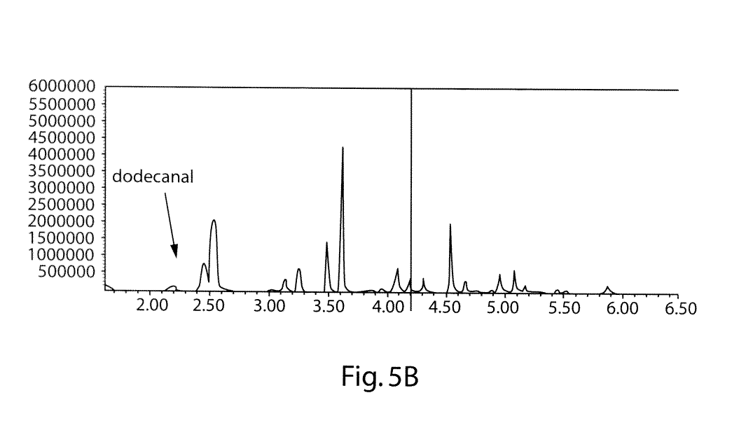

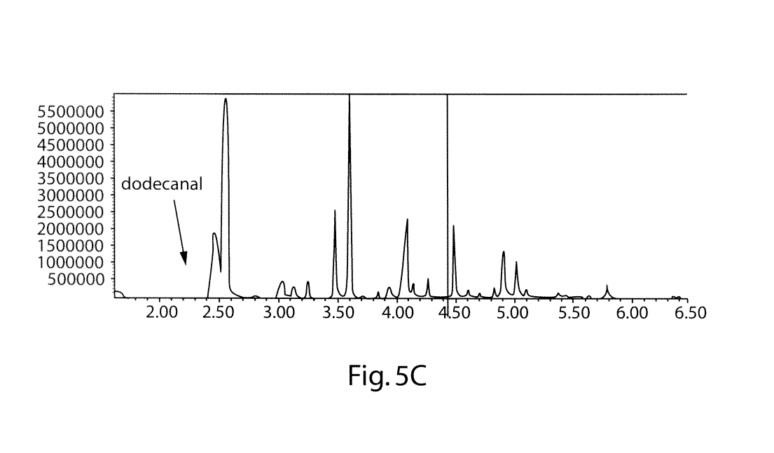

The yjgB knockout strain resulted in significant accumulation of dodecanal and a lower fatty alcohol titer (FIG. 5). The expression of yjgB from plasmid pETDuet-1-′tesA-yjgB in the yjgB knockout strain effectively removed the accumulation of dodecanal (FIG. 5). The data show that YjgB was involved in converting dodecanal into dodecanol and that there is more than one dehydrogenase present in E. coli convert aldehydes into alcohols. Dodecanal accumulated in the yjgB knockout strain, but it was not observed in either the wild-type strain (MG1655(DE3, ΔfadD)) or the yjgB knockout strain with the yjgB expression plasmid. The arrows (in FIG. 5) indicate the GC trace of dodecanal (C12:0 aldehyde).

### Example 6

### Production of Fatty Alcohol by Heterologous Expression of Thioesterase, Acyl-CoA Synthase, and Acyl-CoA Reductase in E. coli C41 (DE3, ΔfadE)

This example demonstrates that expression of a thioesterase gene, an acyl CoA synthase gene, and an acyl-CoA reductase gene in bacteria lacking acyl-CoA dehydrogenase results in the production of fatty alcohol.

The fadD gene (acyl-CoA synthase gene accession NP_{—}416319, EC 6.2.1.3) from E. coli was amplified by PCR using the primers fadD-F and fadD-R (see Table 7). The PCR product was cloned into a NocI/HindIII digested pCDFDuet-1 derivative, which contained the acr1 gene (acyl-CoA reductase gene accession YP_{—}047869) from Acinetobacter sp. ADP1 within its NdeI/AvrII sites.

Plasmids pCDFDuet-1-fadD-acr1 (acyl CoA synthase and acyl-CoA reductase) and pETDuet-1-′tesA (thioesterase) (described in Example 2) were co-transformed into E. coli C41(DE3, ΔfadE). The transformants were selected on LB plates supplemented with 100 mg/L of spectinomycin and 50 mg/L of carbenicillin. Four colonies of E. coli C41(DE3. ΔfadE)/pCDFDuet-1-fadD-acr1/pETDuet-1 tesA were independently inoculated into 3 mL of M9 medium supplemented with 50 mg/L of carbenicillin and 100 mg/L of spectinomycin and grown at 25° C. with shaking (250 rpm) until they reached 0.5 OD₆₀₀. Next, 1.5 mL of each culture was transferred into a 250 mL flask containing 30 mL of the M9 medium described above. The resulting cultures were grown at 25° C. with shaking until the culture reached between 0.5-1.0 OD₆₀₀. IPTG was then added to a final concentration of 1 mM, and the cultures were incubated at 25° C. with shaking for an additional 40 hours.

The cells were then spun down at 4000 rpm, and the cell pellets were suspended in 1.0 mL of methanol. 3 mL of ethyl acetate was then mixed with the suspended cells, followed by 3 mL of H₂O, and the mixture was sonicated for 20 minutes. The resulting sample was centrifuged at 4000 rpm for 5 minutes.

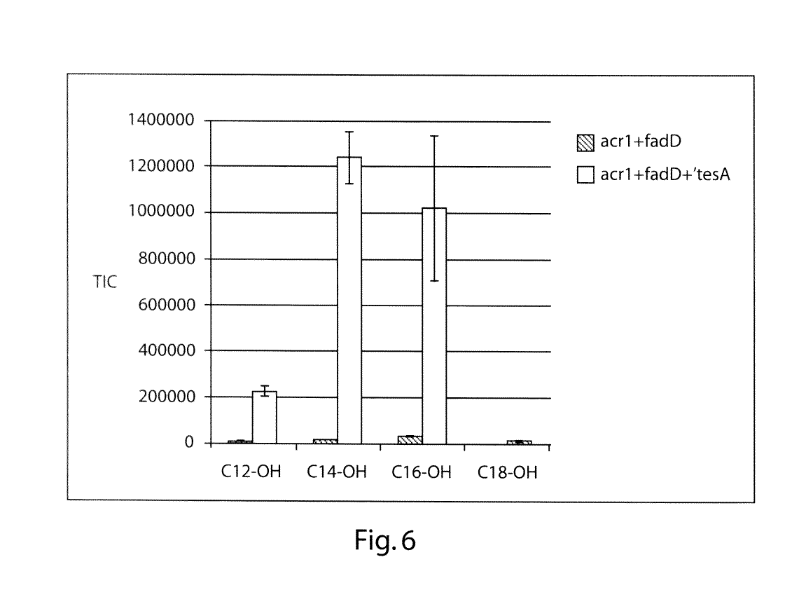

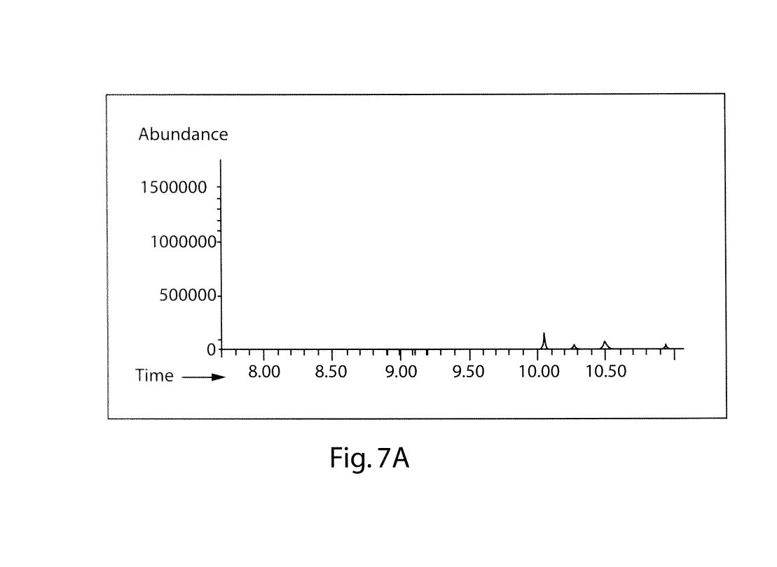

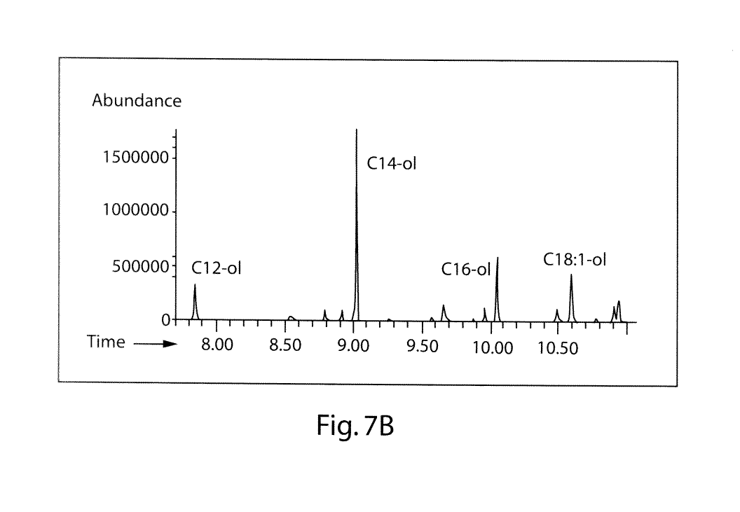

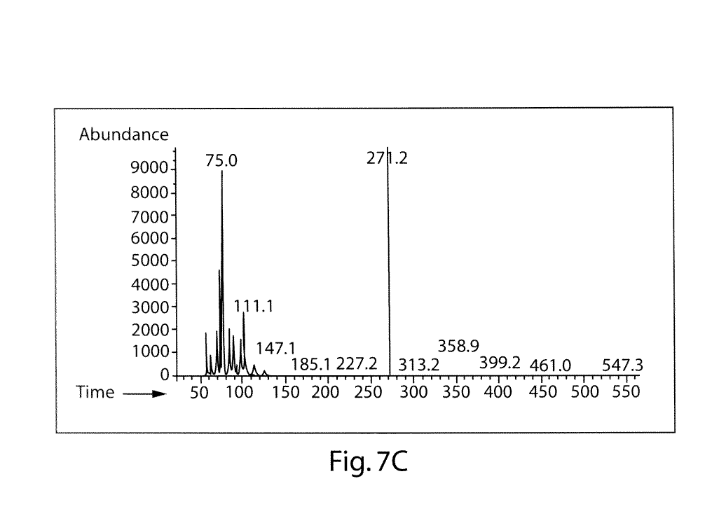

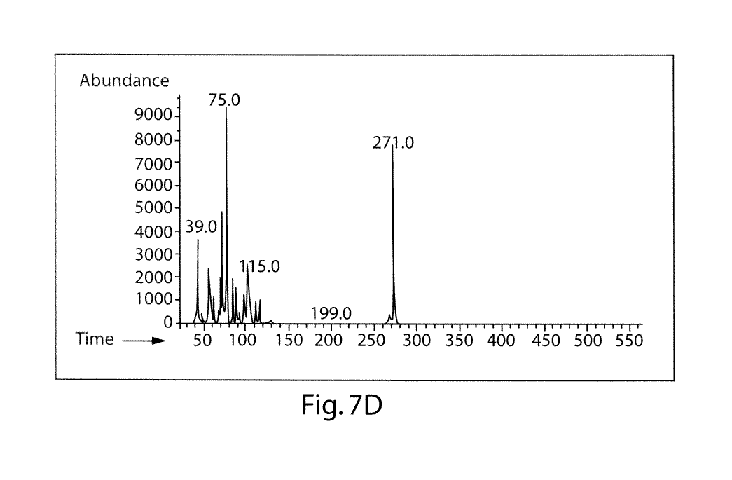

The organic phase (the upper phase), which contained fatty alcohols, was reacted with trimethylsilane (TMS) imidazole by adding 1/5 volume of reagent and then subjected to GC/MS analysis as described herein. The total fatty alcohol yield (including tetradecanol, hexadecanol, hexadecenol and octadecenol) was about 1-10 mg/L (See FIGS. 6 and 7).

As a control, pETDuet-1 empty vector was transformed into E. coli C41(DE3, ΔfadE)/pCDFDuet-1-fadD-acr1. The E. coli C41(DE3, ΔfadE)/pCDFDuet-1-fadD-acr1+pETDuet-1 transformants were cultured and induced with IPTG as described above for E. coli C41(DE3, ΔfadE)/pCDFDuet-1-fadD-acr1/pETDuet-1-′tesA.

The cells were then spun down at 4000 rpm, and the cell pellets were suspended in 1.0 mL of methanol. 3 mL of ethyl acetate was then mixed with the suspended cells, followed by 3 mL of H₂O, and the mixture was sonicated for 20 minutes. The resulting sample was centrifuged at 4000 rpm for 5 minutes.

The organic phase (the upper phase), which contained fatty alcohol, was reacted with trimethylsilane (TMS) imidazole by adding 1/5 volume of reagent and then subjected to GC/MS analysis as described herein. In contrast to the 1-10 mg/L of total alcohol produced by E. coli C41(DE3, ΔfadE)/pCDFDuet-1-fadD-acr1/pETDuet-1-′tesA, the E. coli C41(DE3, ΔfadE)/pCDFDuet-1-fadD-acr1+pETDuet-1 control produced total fatty alcohol yields of only 0.2-0.5 mg/L (FIGS. 6 and 7).

The results of the experiments reflected in this example demonstrate that expression of a thioesterase gene, acyl-CoA synthase, and an acyl-CoA reductase gene in E. coli C4|(DE3. ΔfadE), which lack acyl-CoA dehydrogenase, results in the production of fatty alcohol.

### Example 7

### Production of Fatty Alcohol Using a Variety of Acyl-CoA Reductases

The results of this example demonstrate fatty alcohol production by expressing a variety of acyl-CoA reductases in E. coli.

Each of four genes encoding fatty acyl-CoA reductases (Table 10) from various sources were codon-optimized for expression in E. coli and synthesized by Codon Devices, Inc. (Cambridge, Mass.). Each of the synthesized genes was cloned as a NdeI-AvrII fragment into pCDF-Duet1-fadD, which had been created by cloning the E. coli fadD gene (amplified with primers fadD-F and fadD-R) into the NcoI-HindIII sites of vector pCDF-Duet1. Each of the plasmids carrying these acyl-CoA reductase genes with E. coli fadD gene was transformed into E. coli strain C41 (DE3) strain (Lucigen, Middleton, Wis.).

TABLE 10
Acyl-CoA Reductases

| Acyl-coA | Protein ID | |
|---|---|---|
| Reductase | Accession Number | Protein Sources |
| mFAR1 | AAH07178 | Mus musculus |
| mFAR2 | AAH55759 | Mus musculus |
| JjFAR | AAD38039 | Simmondsia chinensis |
| BmFAR | BAC79425 | Bombyx mori |
| Acr1 | AAC45217 | Acinetobacter baylyi |
| | | ADP1 |
| AcrM | BAB85476 | Acinetobacter sp. M1 |
| hFAR | AAT42129 | Homo sapiens |

The transformants were grown in 3 mL of LB broth supplemented with 100 mg/L of spectinomycin at 37° C. overnight. 0.5 mL of the overnight culture was transferred to 50 mL of fresh M9 medium with 100 mg/L of spectinomycin and grown at 25° C. When the cultures reached an OD₆₀₀ of 0.6-0.7, IPTG was added to obtain a final concentration of 1 mM. Each culture was fed 0.1% of one of three fatty acids dissolved in H₂O at pH 9.0. The three fatty acids fed were sodium dodecanoate, sodium myristate, or palmitic acid. A culture without the addition of fatty acid was also included as a control. After induction, the cultures were grown overnight at 25° C.

The identification of fatty alcohol yield at the end of fermentation was performed using GC-MS as described herein. The resulting fatty alcohol produced from the corresponding fatty acid is shown in Table 10. The results showed that three acyl-CoA reductases—Acr1, AcrM and BmFAR—could convert all three fatty acids into corresponding fatty alcohols. The results also showed that hFAR and JjFAR had activity when myristate and palmitate were the substrates. However, there was little to no activity when dodecanoate was the substrate. mFAR1 and mFAR2 only showed low activity with myristate and showed no activity with the other two fatty acids.

TABLE 11
Fatty Alcohol Production

| | Acyl-CoA | Peak Area | | |
|---|---|---|---|---|
| E coil | Reductase | Dodecanoate/ | Myristate/ | Palmitate/ |
| C41(DE3) | Genes | Dodecanol^{b} | Tetradecanol^{b} | Hexadecanol^{b} |
| | mFAR1 | 7,400 | 85,700 | 8,465 |
| | mFAR2 | 2,900 | 14,100 | 32,500 |
| | JjFAR | 5,200 | 8,500 | 53,112 |
| | BmFAR | 35,800 | 409,000 | 407,000 |
| | acr1 | 202,000 | 495,000 | 1,123,700 |
| | acrM | 42,500 | 189,000 | 112,448 |
| | hFAR1 | 5,050 | 59,500 | 109,400 |

| | | | |
|---|---|---|---|
| Vector Control | 4,000 | 1,483 | 32,700 |
| Media Control | 10,700 | 1,500 | 25,700 |

Notes:
^{a}Only hexadecanol was quantified in this case.
^{b}Fatty acid fed/fatty alcohol produced.
^{c}The area peak of fatty alcohol produced.

The results of the experiments reflected in this example demonstrate that expression of a variety of acyl-CoA reductases in E. coli results in the production of fatty alcohols. In addition, the results of the experiments reflected in this example demonstrate that the type and the quantity of fatty alcohol production varies depending on the specific acyl CoA reductase expressed and the specific type of fatty alcohol that is fed.

### Example 8

### Granular Laundry Detergent Composition Formulations

| | A | B | C | D | E |
|---|---|---|---|---|---|
| Formula | wt % | wt % | wt % | wt % | wt % |
| The surfactant composition of | 5-25 | 5-25 | 13-25 | 13-25 | 9-25 |
| Example 1 | | | | | |
| C₁₂₋₁₈ Ethoxylate Sulfate | — | — | 0-3 | — | 0-1 |
| C₁₄₋₁₅ alkyl ethoxylate | 0-3 | 0-3 | — | 0-5 | 0-3 |
| (EO = 7) | | | | | |
| Dimethyl hydroxyethyl lauryl | — | — | 0-2 | 0-2 | 0-2 |
| ammonium chloride | | | | | |
| Sodium tripolyphosphate | 20-40 | — | 18-33 | 12-22 | 0-15 |
| Zeolite | 0-10 | 20-40 | 0-3 | — | — |
| Silicate builder | 0-10 | 0-10 | 0-10 | 0-10 | 0-10 |
| Carbonate | 0-30 | 0-30 | 10-30 | 15-25 | 0-20 |
| Diethylene triamine penta | 0-1 | 0-1 | 0-1 | 0-1 | 0-1 |
| acetate | | | | | |
| Polyacrylate | 0-3 | 0-3 | 0-3 | 0-3 | 0-3 |
| Carboxy Methyl Cellulose | 0.2-0.8 | 0.2-0.8 | 0.2-0.8 | 0.2-0.8 | 0.2-0.8 |
| Polymer¹ | 0-4 | 0.05-10 | 3.0 | 2.5 | 1.0 |
| Percarbonate | 0-10 | 0-10 | 0-10 | 0-10 | 0-10 |
| Nonanoyloxybenzenesulfonate | — | — | 0-2 | 0-2 | 0-2 |
| Tetraacetylethylenediamine | — | — | 0-0.6 | 0-0.6 | 0-0.6 |
| Zinc Phthalocyanine | — | — | 0-0.005 | 0-0.005 | 0-0.005 |
| Tetrasulfonate | | | | | |
| Brightener | 0.05-0.2 | 0.05-0.2 | 0.05-0.2 | 0.05-0.2 | 0.05-0.2 |
| MgSO₄ | — | — | 0-0.5 | 0-0.5 | 0-0.5 |
| Enzymes | 0-0.5 | 0-0.5 | 0-0.5 | 0-0.5 | 0-0.5 |
| Minors (perfume, dyes, suds | balance | balance | balance | balance | balance |
| stabilizers) | | | | | |

¹An amphiphilic alkoxylated polyalkylenimine polymers or PEG-PVAc graft copolymer

### Example 9

### Preparation of a Spray Dried Powder

### Aqueous Slurry Composition.

| | % w/w |
|---|---|
| | Aqueous |
| Component | slurry |
| A compound having the following general structure: | 1.23 |
| bis((C₂H₅O)(C₂H₄O)n)(CH₃)—N⁺CₓH₂ₓ—N⁺—(CH₃)- | |
| bis((C₂,H₅O)(C₂H₄O)n), wherein n = from 20 | |
| to 30, and x = from 3 to 8, or sulphated or | |
| sulphonated variants thereof | |
| Ethylenediamine disuccinic acid | 0.35 |
| Brightener | 0.12 |
| Magnesium sulphate | 0.72 |
| Acrylate/maleate copolymer | 6.45 |
| Polymer¹ | 1.60 |
| Linear alkyl benzene sulphonate | 11.92 |
| Hydroxyethane di(methylene phosehonic acid) | 0.32 |
| Sodium carbonate | 4.32 |
| Sodium sulphate | 47.49 |
| Soap | 0.78 |
| Water | 24.29 |
| Miscellaneous | 0.42 |
| Total Parts | 100.00 |

¹An amphiphilic alkoxylated polyalkylenimine polymer or any mixture of polymers according to any of Examples 1, 2, 3, or 4.

An aqueous slurry having the composition as described above is prepared having a moisture content of 25.89%. The aqueous slurry is heated to 72° C. and pumped under high pressure (from 5.5×10⁶ Nm⁻² to 6.0×10⁶Nm²), into a counter current spray-drying tower with an air inlet temperature of from 270° C. to 300° C. The aqueous slurry is atomised and the atomised slurry is dried to produce a solid mixture, which is then cooled and sieved to remove oversize material (>1.8 mm) to form a spray-dried powder, which is free-flowing. Fine material (<0.15 mm) is elutriated with the exhaust the exhaust air in the spray-drying tower and collected in a post tower containment system. The spray-dried powder has a moisture content of 1.0 wt %, a bulk density of 427 g/l and a particle size distribution such that 95.2 wt % of the spray-dried powder has a particle size of from 150 to 710 micrometers. The composition of the spray-dried powder is given below.

### Spray-Dried Powder Composition.

| | % w/w Spray- |
|---|---|
| Component | dried powder |
| Ethylenediamine disuccinic acid | 0.46 |
| Brightener | 0.16 |
| Magnesium sulphate | 0.95 |
| Acrylate/maleate copolymer | 8.45 |
| Polymer¹ | 2.09 |
| Linear alkyl benzene sulphonate blend with | 12.65 |
| Example III ratio 2:1 | |
| Hydroxyethane di(methylene phosphonic acid) | 0.42 |
| Sodium carbonate | 5.65 |
| Sodium sulphate | 61.98 |
| Soap | 1.02 |
| Water | 1.00 |
| Miscellaneous | 0.55 |
| Total Parts | 100.00 |

¹An amphiphilic alkoxylated polyalkylenimine polymer or PEG-PVAc graft copolymer

### Example 10

### Preparation of an Anionic Surfactant Particle 1

The anionic detersive surfactant particle 1 is made on a 520 g batch basis using a Tilt-A-Pin then Tilt-A-Plow mixer (both made by Processall). 108 g sodium sulphate supplied is added to the Tilt-A-Pin mixer along with 244 g sodium carbonate, 168 g of 70% active C₂₅E₃S paste (sodium ethoxy sulphate based on C_{12/15} alcohol and ethylene oxide) is added to the Tilt-A-Pin mixer. The components are then mixed at 1200 rpm for 10 seconds. The resulting powder is then transferred into a Tilt-A-Plow mixer and mixed at 200 rpm for 2 minutes to form particles. The particles are then dried in a fluid bed dryer at a rate of 25001/min at 120° C. until the equilibrium relative humidity of the particles is less than 15%. The dried particles are then sieved and the fraction through 1180 μm and on 250 μm is retained The composition of the anionic detersive surfactant particle 1 is as follows:

25.0% w/w C₂₅E₃S sodium ethoxy sulphate

18.0% w/w sodium sulphate

57.0% w/w sodium carbonate

### Example 11

### Preparation of a Cationic Detersive Surfactant Particle 1

The cationic surfactant particle 1 is made on a 14.6 kg batch basis on a Morton FM-50 Loedige mixer. 4.5 kg of micronised sodium sulphate and 4.5 kg micronised sodium carbonate are premixed in the Morton FM-50 Loedige mixer. 4.6 kg of 40% active mono-C₁₂₋₁₄ alkyl mono-hydroxyethyl di-methyl quaternary ammonium chloride (cationic surfactant) aqueous solution is added to the Morton FM-50 Loedige mixer whilst both the main drive and the chopper are operating. After approximately two minutes of mixing, a 1.0 kg 1:1 weight ratio mix of micronised sodium sulphate and micronised sodium carbonate is added to the mixer. The resulting agglomerate is collected and dried using a fluid bed dryer on a basis of 2500 l/min air at 100-140° C. for 30 minutes. The resulting powder is sieved and the fraction through 1400 μm is collected as the cationic surfactant particle 1. The composition of the cationic surfactant particle 1 is as follows:

15% w/w mono-C₁₂₋₁₄ alkyl mono-hydroxyethyl di-methyl quaternary ammonium chloride

40.76% w/w sodium carbonate

40.76% w/w sodium sulphate

3.48% w/w moisture and miscellaneous

### Example 12

### Preparation of a Granular Laundry Detergent Composition

10.84 kg of the spray-dried powder of Example 9, 4.76 kg of the anionic detersive surfactant particle 1 of Example 10, 1.57 kg of the cationic detersive surfactant particle 1 of Example 11, and 7.83 kg (total amount) of other individually dosed dry-added material are dosed into a 1 m diameter concrete batch mixer operating at 24 rpm. Once all of the materials are dosed into the mixer, the mixture is mixed for 5 minutes to form a granular laundry detergent composition. The formulation of the granular laundry detergent composition is described below:

### A Granular Laundry Detergent Composition.

| Component | % w/w | % w/w |
|---|---|---|
| Spray-dried powder from | 43.34 | 15 |
| the “Spray-Dried Powder” section | | |
| (above) | | |
| 91.6 wt % active linear alkyl benzene | 0.22 | 2 |
| sulphonate flake supplied by Stepan | | |
| under the tradename NACCONOL 90G ® | | |
| Citric acid | 5.00 | 0 |
| Sodium percarbonate (having from 12% | 14.70 | 0 |
| to 15% washing active oxygen (active | | |
| AvOx)) | | |
| Photobleach particle | 0.01 | 0 |
| Lipase (11.00 mg active/g) | 0.70 | 0.90 |
| Amylase (21.55 mg active/g) | 0.33 | 0.50 |
| Protease (56.00 mg active/g) | 0.43 | 0.60 |
| Tetraacetyl ethylene diamine | 4.35 | 4.0 |
| agglomerate (92 wt % active) | | |
| Suds suppressor agglomerate | 0.87 | 1.0 |
| (11.5 wt % active) | | |
| Acrylate/maleate copolymer particle | 0.29 | 0 |
| (95.7 wt % active) | | |
| Green/Blue carbonate speckle | 0.50 | 0 |
| Anionic detersive surfactant particle 1 | 19.04 | 10 |
| Cationic detersive surfactant particle 1 | 6.27 | |
| Sodium sulfate | balance | balance |
| Solid perfume particle | 0.63 | 0.7 |
| Total Parts | 100.00 | 100.00 |

### Example 13

### Liquid Laundry Detergents

| | A | B | C | D | E |
|---|---|---|---|---|---|
| Ingredient | wt % | wt % | wt % | wt % | wt % |
| C12-15 EO_{1.8} sulfate sodium salt | 14.4 | 0 | 9.2 | 5.4 | 0 |
| according to the present invention | | | | | |
| Alcohol sulfate according to the present | 4.4 | 12.2 | 5.7 | 1.3 | 20 |
| invention, monoethanolamine salt | | | | | |
| Alkyl ethoxylate | 2.2 | 8.8 | 8.1 | 3.4 | 0 |
| Amine oxide | 0.7 | 1.5 | 0 | 0 | 0 |
| Citric acid | 2.0 | 3.4 | 1.9 | 1.0 | 1.6 |
| HLAS (linear alkylbenzene | 3.0 | 0 | 0 | 0 | 5.0 |
| sulfonate, acid form) | | | | | |
| Protease | 1.0 | 0.7 | 1.0 | 0 | 2.5 |
| Amylase | 0.2 | 0.2 | 0 | 0 | 0.3 |
| Lipase | 0 | 0 | 0.2 | 0 | 0 |
| Borax | 1.5 | 2.4 | 2.9 | 0 | 0 |
| Calcium and sodium formate | 0.2 | 0 | 0 | 0 | 0 |
| Formic acid | 0 | 0 | 0 | 0 | 1.1 |
| Ethoxylated polyamine polymer | 1.7 | 2.0 | | 0.8 | 0 |
| or polymers | | | | | |
| Sodium polyacrylate copolymer | 0 | 0 | 0.6 | 0 | 0 |
| DTPA¹ | 0.1 | 0 | 0 | 0 | 0.9 |
| DTPMP² | 0 | 0.3 | 0 | 0 | 0 |
| EDTA³ | 0 | 0 | 0 | 0.1 | 0 |
| Fluorescent whitening agent | 0.15 | 0.2 | 0.12 | 0.12 | 0.2 |
| Ethanol | 2.5 | 1.4 | 1.5 | 0 | 0 |
| Propanediol | 6.6 | 4.9 | 4.0 | 0 | 15.7 |
| Sorbitol | 0 | 0 | 4.0 | 0 | 0 |
| Ethanolamine | 1.5 | 0.8 | 0.1 | 0 | 11.0 |
| Sodium hydroxide | 3.0 | 4.9 | 1.9 | 1.0 | 0 |
| Sodium cumene sulfonate | 0 | 2.0 | 0 | 0 | 0 |
| Silicone suds suppressor | 0 | 0.01 | 0 | 0 | 0 |
| Perfume | 0.3 | 0.7 | 0.3 | 0.4 | 0.6 |
| Opacifier⁴ | 0 | 0.30 | 0.20 | 0 | 0.50 |
| Water | balance | balance | balance | balance | balance |
| | 100.0% | 100.0% | 100.0% | 100.0% | 100.0% |

¹diethylenetriaminepentaacetic acid, sodium salt
²diethylenetriaminepentakismethylenephosphonic acid, sodium salt
³ethylenediaminetetraacetic acid, sodium salt
⁴Acusol OP 301

| | F | G | H | I | J | K |
|---|---|---|---|---|---|---|
| Ingredient | wt % | wt % | wt % | wt % | wt % | wt % |
| Alkylbenzene sulfonic acid | 7 | 7 | 4.5 | 1.2 | 1.5 | 12.5 |
| Sodium C12-14 alkyl ethoxy | 2.3 | 2.3 | 4.5 | 4.5 | 7 | 18 |
| 3 sulfate | | | | | | |
| The alcohol ethoxylate of | 5 | 5 | 2.5 | 2.6 | 4.5 | 4 |
| Example II | | | | | | |
| C12 alkyl dimethyl amine | — | 2 | — | — | — | — |
| oxide | | | | | | |
| C12-14 alkyl hydroxyethyl | — | — | — | 0.5 | — | — |
| dimethyl ammonium chloride | | | | | | |
| C12-18 Detergent acid | 2.6 | 3 | 4 | 2.6 | 2.8 | 11 |
| Citric acid | 2.6 | 2 | 1.5 | 2 | 2.5 | 3.5 |
| Protease enzyme | 0.5 | 0.5 | 0.6 | 0.3 | 0.5 | 2 |
| Amylase enzyme | 0.1 | 0.1 | 0.15 | — | 0.05 | 0.5 |
| Mannanase enzyme | 0.05 | — | 0.05 | — | — | 0.1 |
| Alkoxylated | 1.0 | .8 | 1 | 0.4 | 1.5 | 2.7 |
| Polyalkylenimine Polymer¹ | | | | | | |
| Diethylenetriaminepenta- | 0.2 | 0.3 | — | — | 0.2 | — |
| (methylenephosphonic) acid | | | | | | |
| Hydroxyethane diphosphonic | — | — | 0.45 | — | — | 1.5 |
| acid | | | | | | |
| FWA | 0.1 | 0.1 | 0.1 | — | — | 0.2 |
| Solvents (1,2 propanediol, | 3 | 4 | 1.5 | 1.5 | 2 | 4.3 |
| ethanol), stabilizers | | | | | | |
| Hydrogenated castor oil | 0.4 | 0.3 | 0.3 | 0.1 | 0.3 | — |
| derivative structurant | | | | | | |
| Boric acid | 1.5 | 2 | 2 | 1.5 | 1.5 | 0.5 |
| Na formate | — | — | — | 1 | — | — |
| Reversible protease inhibitor³ | — | — | 0.002 | — | — | — |
| Perfume | 0.5 | 0.7 | 0.5 | 0.5 | 0.8 | 1.5 |

| | |
|---|---|
| Buffers (sodium hydroxide, | To pH 8.2 |
| Monoethanolamine) | |
| Water and minors | To 100 |

¹Amphiphilic alkoxylated polyalkylenimine polymer or any mixture of polymers according to any of Examples 1, 2, 3, or 4.

| | L | M | N | O | P | Q |
|---|---|---|---|---|---|---|
| Ingredient | wt % | wt % | wt % | wt % | wt % | wt % |
| Alkylbenzene sulfonic acid | 5.5 | 2.7 | 2.2 | 12.2 | 5.2 | 5.2 |
| The alcohol sulfate of | 16.5 | 20 | 9.5 | 7.7 | 1.8 | 1.8 |
| Example I | | | | | | |
| Sodium C12-14 alkyl sulfate | 8.9 | 6.5 | 2.9 | — | | |
| C12-14 alkyl 7-ethoxylate | | | | | 0.15 | 0.15 |
| C14-15 alkyl 8-ethoxylate | | | | | 3.5 | 3.5 |
| C12-15 alkyl 9-ethoxylate | 1.7 | 0.8 | 0.3 | 18.1 | — | — |
| C12-18 Detergent acid | 2.2 | 2.0 | — | 1.3 | 2.6 | 2.6 |
| Citric acid | 3.5 | 3.8 | 2.2 | 2.4 | 2.5 | 2.5 |
| Protease enzyme | 1.7 | 1.4 | 0.4 | — | 0.5 | 0.5 |
| Amylase enzyme | 0.4 | 0.3 | — | — | 0.1 | 0.1 |
| Mannanase enzyme | | | | | 0.04 | 0.04 |
| Alkoxylated | 2.1 | 1.2 | 1.0 | 2 | 1.00 | 0.25 |
| Polyalkylenimine Polymer¹ | | | | | | |
| PEG-PVAc Polymer² | — | — | — | — | — | 0.3 |
| Ethoxysulfated | — | — | — | — | — | 0.7 |
| Hexamethylene Diamine | | | | | | |
| Dimethyl Quat | | | | | | |
| Diethylenetriaminepenta | | | | | 0.2 | 0.2 |
| (methylenephosphonic) acid | | | | | | |
| FWA | — | — | — | — | .04 | .04 |
| Solvents (1,2 propanediol, | 7 | 7.2 | 3.6 | 3.7 | 1.9 | 1.9 |
| ethanol, stabilizers | | | | | | |
| Hydrogenated castor oil | 0.3 | 0.2 | 0.2 | 0.2 | 0.35 | 0.35 |
| derivative structurant | | | | | | |
| Polyacrylate | — | — | — | 0.1 | — | — |
| Polyacrylate copolymer³ | — | — | — | 0.5 | — | — |
| Sodium carbonate | — | — | — | 0.3 | — | — |
| Sodium silicate | — | — | — | — | — | — |
| Borax | 3 | 3 | 2 | 1.3 | — | — |
| Boric acid | 1.5 | 2 | 2 | 1.5 | 1.5 | 1.5 |
| Perfume | 0.5 | 0.5 | 0.5 | 0.8 | 0.5 | 0.5 |
| Buffers (sodium hydroxide, | | | | | 3.3 | 3.3 |
| monoethanolamine) | | | | | | |

| Water, dyes and | Balance |
|---|---|
| miscellaneous | |
| ¹Amphiphilic alkoxylated polyalkylenimine polymer or any mixture of polymers according to any of Examples 1, 2, 3, or 4. | |
| ²PEG-PVA graft copolymer is a polyvinyl acetate grafted polyethylene oxide copolymer having a polyethylene oxide backbone and multiple polyvinyl acetate side chains. The molecular weight of the polyethylene oxide backbone is about 6000 and the weight ratio of the polyethylene oxide to polyvinyl acetate is about 40 to 60 and no more than 1 grafting point | |
| per 50 ethylene oxide units. | |

³Alco 725 (styrene/acrylate)

### Example Liquid Laundry Detergent

| Ingredient | Wt % |
|---|---|
| Propylene glycol | 4.75 |
| Sodium citrate | 2.8 |
| NaOH (50%) | 0.43 |
| Monoethanolamine | 0.23 |
| LAS, acid form | 6.0 |
| Coconut fatty acid | 0.77 |
| REO2 sulfate, wherein R is | 10.5 |
| according to the present invention | |
| Nonionic surfactant | 6.6 |
| 1-decanol | 6.0 |
| Protease | 0.45 |
| Lipase | 0.25 |
| Perfume | 0.2 |
| Water | Balance to 100 |

### Example 14

### Liquid Dish Handwashing Detergents

| Composition | A | B |
|---|---|---|
| C₁₂₋₁₃ Natural AE0.6S | 270 | 240 |
| C₁₀₋₁₄ Amine Oxide | — | 6.0 |
| The alcohol ethoxylated | 2.0 | 5.0 |
| sulfate of example V | | |
| C₁₂₋₁₄ Linear Amine Oxide | 6.0 | — |
| SAFOL ® 23 Amine Oxide | 1.0 | 1.0 |
| C₁₁E₉ Nonionic² | 2.0 | 2.0 |
| Ethanol | 4.5 | 4.5 |
| Polymer¹ | 5.0 | 2.0 |
| Sodium cumene sulfonate | 1.6 | 1.6 |
| Polypropylene glycol 2000 | 0.8 | 0.8 |
| NaCl | 0.8 | 0.8 |
| 1,3 BAC Diamine³ | 0.5 | 0.5 |
| Suds boosting polymer⁴ | 0.2 | 0.2 |
| Water | Balance | Balance |

¹An amphiphilic alkoxylated polyalkylenimine polymer or any mixture of polymers according to any of Examples 1. 2, 3, or 4.
²Nonionic may be either C₁₁ Alkyl ethoxylated surfactant containing 9 ethoxy groups.
³1,3, BAC is 1,3 bis(methylamine)-cyclohexane.
⁴(N,N-dimethylamino)ethyl methacrylate homopolymer

### Example 15

### Automatic Dishwasher Detergent

| | A | B | C | D | E |
|---|---|---|---|---|---|
| Polymer dispersant² | 0.5 | 5 | 6 | 5 | 5 |
| Carbonate | 35 | 40 | 40 | 35-40 | 35-40 |
| Sodium | 0 | 6 | 10 | 0-10 | 0-10 |
| tripolyphosphate | | | | | |
| Silicate solids | 6 | 6 | 6 | 6 | 6 |
| Bleach and bleach | 4 | 4 | 4 | 4 | 4 |
| activators | | | | | |
| Polymer¹ | 0.05-10 | 1 | 2.5 | 5 | 10 |
| Enzymes | 0.3-0.6 | 0.3-0.6 | 0.3-0.6 | 0.3-0.6 | 0.3-0.6 |
| Disodium citrate | 0 | 0 | 0 | 2-20 | 0 |
| dihydrate | | | | | |
| Nonionic surfactant | 0-2 | 0-1 | 0-1 | 0-1.5 | 0.8-5 |
| of example IV | | | | | |
| Water, sulfate, | Balance | Balance | Balance | Balance | Balance |
| perfume, dyes and | | | | | |
| other adjuncts | to 100% | to 100% | to 100% | to 100% | to 100% |

¹An amphiphilic alkoxylated polyalkylenimine polymer or any mixture of polymers according to any of Examples 1, 2, 3, or 4.
²Such as ACUSOL ® 445N available from Rohm & Haas or ALCOSPERSE ® from Alco.

### Hard Surface Cleaner

A hard surface cleaner comprises 5% total nonionic surfactant (ROH according to the present invention ethoxylated with 8 moles of ethylene oxide), 0.2% citric acid, perfume 0.3%, and water to 100%.

Unless otherwise noted, all component or composition levels are in reference to the active level of that component or composition, and are exclusive of impurities, for example, residual solvents or by-products, which may be present in commercially available sources.

All percentages and ratios are calculated by weight unless otherwise indicated. All percentages and ratios are calculated based on the total composition unless otherwise indicated.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited herein are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

SEQUENCE LISTINGS 1:
The nucleotide sequence and the corresponding amino acid sequence of
Nocardia sp. NRRL 5646 car gene
AAR91681.1
Nucleotide sequence
>gi|40796034:488-4012 Nocardia sp. NRRL 5646 ATP/NADPH-dependent
carboxylic acid reductase (car) gene, complete cds
(SEQ ID NO: 15)
ATGGCAGTGGATTCACCGGATGAGCGGCTACAGCGCCGCATTGCACAGTTGTTTGCAGAAGATGAGCAGG
TCAAGGCCGCACGTCCGCTCGAAGCGGTGAGCGCGGCGGTGAGCGCGCCCGGTATGCGGCTGGCGCAGAT
CGCCGCCACTGTTATGGCGGGTTACGCCGACCGCCCGGCCGCCGGGCAGCGTGCGTTCGAACTGAACACC
GACGACGCGACGGGCCGCACCTCGCTGCGGTTACTTCCCCGATTCGAGACCATCACCTATCGCGAACTGT
GGCAGCGAGTCGGCGAGGTTGCCGCGGCCTGGCATCATGATCCCGAGAACCCCTTGCGCGCAGGTGATTT
CGTCGCCCTGCTCGGCTTCACCAGCATCGACTACGCCACCCTCGACCTGGCCGATATCCACCTCGGCGCG
GTTACCGTGCCGTTGCAGGCCAGCGCGGCGGTGTCCCAGCTGATCGCTATCCTCACCGAGACTTCGCCGC
GGCTGCTCGCCTCGACCCCGGAGCACCTCGATGCGGCGGTCGAGTGCCTACTCGCGGGCACCACACCGGA
ACGACTGGTGGTCTTCGACTACCACCCCGAGGACGACGACCAGCGTGCGGCCTTCGAATCCGCCCGCCGC
CGCCTTGCCGACGCGGGCAGCTTGGTGATCGTCGAAACGCTCGATGCCGTGCGTGCCCGGGGCCGCGACT
TACCGGCCGCGCCACTGTTCGTTCCCGACACCGACGACGACCCGCTGGCCCTGCTGATCTACACCTCCGG
CAGCACCGGAACGCCGAAGGGCGCGATGTACACCAATCGGTTGGCCGCCACGATGTGGCAGGGGAACTCG
ATGCTGCAGGGGAACTCGCAACGGGTCGGGATCAATCTCAACTACATGCCGATGAGCCACATCGCCGGTC
GCATATCGCTGTTCGGCGTGCTCGCTCGCGGTGGCACCGCATACTTCGCGGCCAAGAGCGACATGTCGAC
ACTGTTCGAAGACATCGGCTTGGTACGTCCCACCGAGATCTTCTTCGTCCCGCGCGTGTGCGACATGGTC
TTCCAGCGCTATCAGAGCGAGCTGGACCGGCGCTCGGTGGCGGGCGCCGACCTGGACACGCTCGATCGGG
AAGTGAAAGCCGACCTCCGGCAGAACTACCTCGGTGGGCGCTTCCTGGTGGCGGTCGTCGGCAGCGCGCC
GCTGGCCGCGGAGATGAAGACGTTCATGGAGTCCGTCCTCGATCTGCCACTGCACGACGGGTACGGGTCG
ACCGAGGCGGGCGCAAGCGTGCTGCTCGACAACCAGATCCAGCGGCCGCCGGTGCTCGATTACAAGCTCG
TCGACGTGCCCGAACTGGGTTACTTCCGCACCGACCGGCCGCATCCGCGCGGTGAGCTGTTGTTGAAGGC
GGAGACCACGATTCCGGGCTACTACAAGCGGCCCGAGGTCACCGCGGAGATCTTCGACGAGGACGGCTTC
TACAAGACCGGCGATATCGTGGCCGAGCTCGAGCACGATCGGCTGGTCTATGTCGACCGTCGCAACAATG
TGCTCAAACTGTCGCAGGGCGAGTTCGTGACCGTCGCCCATCTCGAGGCCGTGTTCGCCAGCAGCCCGCT
GATCCGGCAGATCTTCATCTACGGCAGCAGCGAACGTTCCTATCTGCTCGCGGTGATCGTCCCCACCGAC
GACGCGCTGCGCGGCCGCGACACCGCCACCTTGAAATCGGCACTGGCCGAATCGATTCAGCGCATCGCCA
AGGACGCGAACCTGCAGCCCTACGAGATTCCGCGCGATTTCCTGATCGAGACCGAGCCGTTCACCATCGC
CAACGGACTGCTCTCCGGCATCGCGAAGCTGCTGCGCCCCAATCTGAAGGAACGCTACGGCGCTCAGCTG
GAGCAGATGTACACCGATCTCGCGACAGGCCAGGCCGATGAGCTGCTCGCCCTGCGCCGCGAAGCCGCCG
ACCTGCCGGTGCTCGAAACCGTCAGCCGGGCAGCGAAAGCGATGCTCGGCGTCGCCTCCGCCGATATGCG
TCCCGACGCGCACTTCACCGACCTGGGCGGCGATTCCCTTTCCGCGCTGTCGTTCTCGAACCTGCTGCAC
GACATCTTCGGGGTCGAGGTGGCGGTGGGTGTCGTCGTCAGCCCGGCGAACGAGCTGCGCGATCTGGCGA
ATTACATTGAGGCGGAACGCAACTCGGGCGCGAAGCGTCCCACCTTCACCTCGGTGCACGGCGGCGGTTC
CGAGATCCGCGCCGCCGATCTGACCCTCGACAAGTTCATCGATGCCCGCACCCTGGCCGCCGCCGACAGC
ATTCCGCACGCGCCGGTGCCAGCGCAGACGGTGCTGCTGACCGGCGCGAACGGCTACCTCGGCCGGTTCC
TGTGCCTGGAATGGCTGGAGCGGCTGGACAAGACGGGTGGCACGCTGATCTGCGTCGTGCGCGGTAGTGA
CGCGGCCGCGGCCCGTAAACGGCTGGACTGGGCGTTCGACAGCGGCGATCGCGGCCTGCTCGAGCACTAC
CAGGAACTGGCCGCACGGACCCTGGAAGTCCTCGCCGGTGATATCGGCGACCCGAATCTCGGTCTGGACG
ACGCGACTTGCCAGCGGTTGGCCGAAACCGTCGACCTGATCGTCCATCCCGCCGCGTTGGTCAACCACGT
CCTTCCCTACACCCAGCTGTTCGGCCCCAATGTCGTCGGCACCGCCGAAATCGTCCGGTTCGCGATCACG
GCGCGGCGCAAGCCGGTCACCTACCTGTCGACCGTCGGAGTGGCCGACCAGGTCGACCCGGCGGAGTATC
AGGAGGACAGCGACGTCCGCGAGATGAGCGCGGTGCGCGTCGTGCGCGAGAGTTACGCCAACCGCTACGG
CAACAGCAAGTGGGCGGGGGAGGTCCTGCTGCGCGAAGCACACGATCTGTGTGGCTTGCCGGTCGCGGTG
TTCCGTTCGGACATGATCCTGGCGCACAGCCGGTACGCGGGTCAGCTCAACGTCCAGGACGTGTTCACCC
GGCTGATCCTCAGCCTGGTCGCCACCGGCATCGCGCCGTACTCGTTCTACCGAACCGACGCGGACGGCAA
CCGGCAGCGGGCCCACTATGACGGCTTGCCGGCGGACTTCACGGCGGCGGCGATCACCGCGCTCGGCATC
CAAGCCACCGAAGGCTTCCGGACCTACGACGTGCTCAATCCGTACGACGATGGCATCTCCCTCGATGAAT
TCGTCGACTGGCTCGTCGAATCCGGCCACCCGATCCAGCGCATCACCGACTACAGCGACTGGTTCCACCG
TTTCGAGACGGCGATCCGCGCGCTGCCGGAAAAGCAACGCCAGGCCTCGGTGCTGCCGTTGCTGGACGCC
TACCGCAACCCCTGCCCGGCGGTCCGCCGCGCGATACTCCCGGCCAAGGAGTTCCAAGCGGCGGTGCAAA
CAGCCAAAATCGGTCCGGAACAGGACATCCCGCATTTGTCCGCGCCACTGATCGATAAGTACGTCAGCGA
TCTGGAACTGCTTCAGCTGCTCTGA
Amino acid sequence
>gi|40796035|gb|AAR91681.1| ATP/NADPH-dependent carboxylic acid reductase
[Nocardia sp. NRRL 5646]
(SEQ ID NO: 16)
MAVDSPDERLQRRIAQLFAEDEQVKAARPLEAVSAAVSAPGMRLAQIAATVMAGYADRPAAGQRAFELNT
DDATGRTSLRLLPRFETITYRELWQRVGEVAAAWHHDPENPLRAGDFVALLGFTSIDYATLDLADIHLGA
VTVPLQASAAVSQLIAILTETSPRLLASTPEHLDAAVECLLAGTTPERLVVFDYHPEDDDQRAAFESARR
RLADAGSLVIVETLDAVRARGRDLPAAPLFVPDTDDDPLALLIYTSGSTGTPKGAMYTNRLAATMWQGNS
MLQGNSQRVGINLNYMPMSHIAGRISLFGVLARGGTAYFAAKSDMSTLFEDIGLVRPTEIFFVPRVCDMV
FQRYQSELDRRSVAGADLDTLDREVKADLRQNYLGGRFLVAVVGSAPLAAEMKTFMESVLDLPLHDGYGS
TEAGASVLLDNQIQRPPVLDYKLVDVPELGYFRTDRPHPRGELLLKAETTIPGYYKRPEVTAEIFDEDGF
YKTGDIVAELEHDRLVYVDRRNNVLKLSQGEFVTVAHLEAVFASSPLIRQIFIYGSSERSYLLAVIVPTD
DALRGRDTATLKSALAESIQRIAKDANLQPYEIPRDFLIETEPFTIANGLLSGIAKLLRPNLKERYGAQL
EQMYTDLATGQADELLALRREAADLPVLETVSRAAKAMLGVASADMRPDAHFTDLGGDSLSALSFSNLLH
EIFGVEVPVGVVVSPANELRDLANYIEAERNSGAKRPTFTSVHGGGSEIRAADLTLDKFIDARTLAAADS
IPHAPVPAQTVLLTGANGYLGRFLCLEWLERLDKTGGTLICVVRGSDAAAARKRLDSAFDSGDPGLLEHY
QQLAARTLEVLAGDIGDPNLGLDDATWQRLAETVDLIVHPAALVNHVLPYTQLFGPNVVGTAEIVRLAIT
ARRKPVTYLSTVGVADQVDPAEYQEDSDVREMSAVRVVRESYANGYGNSKWAGEVLLREAHDLCGLPVAV
FRSDMILAHSRYAGQLNVQDVFTRLILSLVATGIAPYSFYRTDADGNRQRAHYDGLPADFTAAAITALGI
QATEGFRTYDVLNPYDDGISLDEFVDWLVESGHPIQRITDYSDWFHRFETAIRALPEKQRQASVLPLLDA
YRNPCPAVRGAILPAKEFQAAVQTAKIGPEQDIPHLSAPLIDKYVSDLELLQLL

SEQUENCE LISTINGS 2:
Nucleotide and amino acid sequences of car homolog genes
ABK75684 (CARA)
Nucleotide sequence
>gi|118168627:3015785-3019291 Mycobacterium smegmatis str. MC2 155,
complete genome
(SEQ ID NO: 19)
TTACAGCAATCCGAGCATCTGCAGGTTGCTGATGTACTTGACGATCACGTCGGCCGTGACGTGCGGAATG
TCCTTGTCGGGGCCGATCTTCGCGTCCTGCACCGCGGCACGGAACCGGTCGGTGGGTGCCATGGCACCGC
ACACGGGCGGTGAGGGCTGCTGATAGTTGTGCAGCAGCGGCAGCAGCGAGGCCTGACGTTGCCGTTCCGG
CAGGGCCCGCAGTGCGGTTTCGAACCGGCTCAGCCAGGTGGCGTAGTCGTCGACGCGGTGCACGGGGTAG
CCGGCCTCGATCAGCCAGTCCACGTACTCGTCGAGGCCGATGCCGTCGTCGTACGGGTTCATCACGTGGA
ACGTCTCGAATCCGTCGGTGACCTGCGAGCCGATGGTGGAGATCGCCTCGGCGATGAACTCCACGGGCAG
CCCGTCGTAGTGGGCGCGCTGCCGGTTGCCGTCCGCATCGAGTTCGTAGAACGAACCGGGCGCGATGCCG
GTCGCCACGAGGCTCAGCATCAGGCGGGTGAACATGTCCGGCAGGTTCAGCTGACCCGAGTAGGTCGTGT
CGGCCAGGATCATGTCGCAGCGGAACACCGAGACCGGCAGACCACACCAGTCGTGCGCCTCCCGCAGCAG
GACCTCGCGGGCCCACTTGCTGTTGCCGTAGCCGTTGGCGTACGAGTCGTCGACCCGGCGCGTCGCGCTG
ATCTCGCCGATGTGGGCGTCCTCGACGAACGCCTCGGGGGAGATGCCCTGTCCCACACCGATCGTCGAGA
CGTACACGTACGGCTTGATCGTGGTGGTCAGCGCGATGCGGATGAGTTCGGCGGTGCCGAGCGCATTGGG
TCCGAACATCTGGCTGTACGGCAGGACGTGATTGACCAGGGCGGCCGGATCGACGATCAGATCGACGGTG
TCGGCCAGTCGCTGCCACGTGTCGTGGTCGAGACCCAGATCGGCCTCGGCCTTGTCACCGGCGATCACCT
CGAGGTGATCGGCTGCCAGCGCGCGGTAGTGCTCGAGGAGTGTCGCGTCCCCGGTGTCGAACGTGGCGTC
CAGACGCGCCCGGGCCTCGTCGTCGCTGCGGGCGCGCACCAGGCAGATCACCTTGCCGTCCACCAGGTCC
ATGCGCTCCAGCCATTCCAGCGCCAGATAGCGGCCCAGGAACCCGGTGGCGCCGGTCAGCAGCACGGTGC
GGATCTCGGTGCCCGAACGCGGCAGACCCGGCGCGGCGGACAGGGTCTTGGCGTCGATGAACTTGCCCAG
GGCGAGATCACGCGCGCGCACCTCGGTGGCGTCGCGCCCGTGCACCGACGCGTATGTGGGGCGCTTGGAG
CCGCGCAGTTCGCCCTCGATGTAGGCCGCGACGCCTGCCAGGTCGGTGGCCGGGCTGACGATGACGCCGA
CCGGCACGTCGACATCGAAGATCTCGTOCAACAGGTTCGAGAAGCTCAAGGCCGACAACGAATCTCCACC
CAGATCGGTGAAGTGCGCATCGGACCGCAGATCCGTGACGGAGCCACCGAGGAGTGCGACCGCGGCGCGG
CTGACGGTCTCGACCACGGGCCGGTCGGCTCCGTTGCGGCGCAACTCGCGCAACTCGTTGGCCTGCCCCT
CGGCCAGGTCGGTGTAGAGCTGTTCGAGGCGTTCGCCGTAGTGCGCCTTCAGTTTCGGCCGGGCCAGCTT
GCGGATACCGGTCAGGAGGCCOTTCTCCAGCGTGAAAGGTGTTGTCTCGACGAGGAAGTCACGCGGGATC
TCATACGACTGCAATCCGGCGGCTCGTGCCGCGTCCTGCAGTGAGTCGCTGATGCGCGACTTGAGTTCGT
CACCGTCCCAACGTCACAGTGCCTCTTCGGTCGGGACCACGACCGCCAGGAGATAGGACCGCGCGCTGTT
GCCGTAGACGTAGATCTGGCGTACCAGGGGGCTGTCGCCGAACACCGCCTCCAGCTTGGAGACCGTGACG
AATTCGCCCTGCGACAGTTTCAGCACGTTGTTGCGGCGGTCGAGGTATTCGAGATGGTCGGGCCCGAGCT
CGGCGACGATGTCGCCGGTGCGGTAGTACCCGTCCTCGTCGAACATCTCGGCGGTGATCTCCGGACGCTT
GTAGTAGCCGGGGAACATCTGCTCGGACTTGACCAGAAGTTCGCCGCGCGGGTAGGGCCGGTCCGTGGCG
AAGTAGCCGAGATCGGGCACGTCGACCAGCTTGTAGTCGATGACCGGCGGGCGCTGGATCTGCCCGTCGA
TGAACACCGCCCCGGCCTCGGTGGAGCCGTAGCCCTCCAGCAGATGCATGTCGAGCAGGTCCTCGACCCA
GCTCTTCATCTCCOCCGAGATGGGAGCCGATCCGGTCAGGGCCGAAACGAATCGCCCGCCGAGCAGTTGG
GTGCGGACCTCTTCGAGGACTGCGGCTTCGGCTCGGTCCTCGGATCCCTCGGCGCGGCGGTTGTCGAGGC
GGCTCTGGTACTCCTGGAACAGCATGTCCCAGATGCGAGGAACCAAGTTGAGCTGCGTGGGCCGCACGAG
GGCGAGGTCCTCCAGGAAGGTGGACAGGTCGCTGCGTGCGGCGAAGTACGCGGTTCCGCCGCTGGCGAGT
GTGCTGCACAGGATGCCGCGCCCCATGACGTGACTCATGGGCATGAAGTTCAGGGTGATCGACCGCATCA
CGCCGAGGGTCTCGTCCCACCGGGCCTTGGACCCGGCCTGCCACATCGTGGCGGTCTTGGACTCGGGGTA
CATCGCGCCCTTGGGAGTGCCGGTGCTGCCGGAGGTGTAGATGAGAAGGGTCAGCGGGTCGGCCTCGTCG
GGCACGTAGAGCGGTGCGTCGGCGAGTGACCGCCCGCGGTCCAGTGCGTCGGTGATCGTCTCGACGACGA
CGCCGGTGCCTGCGAGCTTGCCCTTGGCCGCCTCGAACGCCTCACGCTGATCGTCGACCTCGTGGCTGTA
GTCGAACACCACCAGTCGCGACGGCGCGGGCCCGGACTCGACGAGAGCGACTGCGTCGGCGAGGAAGTCG
ACGCTCGACGCGATCACCTTGGGCTCGGTCTCGGCGACGATCGGCTGCAGTTGGGCCACCGGCGCACTGG
TCTGCAGCGGTACGGACACGGCGCCGAGTTCGAGCAGGGCGATGTCGATCGTCGTGTAGTCGACACTGGT
GAAACCCAGGATGGCCACGCGGTCACCGGCATTCACCGGATGGTTGTGCCAGGCATTGGTCACGGCCTGG
ATCCGGCCTGCGAGCTGACGGTAGGTGATGGTGTCGAAGCGGGGCAGGAGCTTCGCGGTGGTGCGGCCTT
CTTCGTCGGTGACGAACTCGACGGCGCGCTTGCCCAGCGCAGGGCGGTCCGCATAGCCGGCCAGAATCTG
TTTGACCGCGGCAGGAAGGCGCAACTCCGGATCGGCGGCAGCCGCGCTGATCGCCTCGTCGGGACGGGCG
GCGGCGAACTGCGGGTCGGTTTCGAACAAGTGGTCAATGCGCCGGTTGAAGCGGTCTTCGCGCGTTTCGA
TCGTCAT
Amino acid sequence
>gi|118174788|gb|ABK75684.1| NAD dependent epimerase/dehydratase family
protein [Mycobacterium smegmatis str. MC2 155]
(SEQ ID NO: 20)
MTIETREDRFNRRIDHLFETDPQFAAARPDEAISAAAADPELRLPAAVKQILAGYADRPALGKRAVEFVT
DEEGRTTAKLLPRFDTITYRQLAGRIQAVTNAWHNHPVNAGDRVAILGFTSVDYTTIDIALLELGAVSVP
LQTSAPVAQLQPIVAETEPKVIASSVDFLADAVALVESGPAPSRLVVFDYSHEVDDQREAFEAAKGKLAG
TGVVVETITDALDRGRSLADAPLYVPDEADPLTLLIYTSGSTGTPKGAMYPESKTATMWQAGSKARWDET
LGVMPSITLNFMPMSHVMGRGILCSTLASGGTAYFAARSDLSTFLEDLALVRPTQLNFVPRIWDMLFQEY
QSRLDNRRAEGSEDRAEAAVLEEVRTQLLGGRFVSALTGSAPISAEMKSWVEDLLDMHLLEGYGSTEAGA
VFIDGQIQRPPVIDYKLVDVPDLGYFATDRPYPRGELLVKSEQMFPGYYKRPEITAEMFDEDGYYRTGDI
VAELGPDHLEYLDRRNNVLKLSQGEFVTVSKLEAVFGDSPLVRQIYVYGNSARSYLLAVVVPTEEALSRW
DGDELKSRISDSLQDAARAAGLQSYEIPRDFLVETTPFTLENGLLTGIRKLARPKLKAHYGERLEQLYTD
LAEGQANELRELRRNGADRPVVETVSRAAVALLGASVTDLRSDAHFTDLGGDSLSALSFSNLLHEIFDVD
VPVGVIVSPATDLAGVAAYIEGELRGSKRPTYASVHGRDATEVRARDLALGKFIDAKTLSAAPGLPRSGT
EIRTVLLTGATGFLGRYLALEWLERMDLVDGKVICLVRARSDDEARARLDATFDTGDATLLEHYRALAAD
HLEVIAGDKGEADLGLDHDTWQRLADTVDLIVDPAALVNHVLPYSQMFGPNALGTAELIRIALTTTIKPY
VYVSTIGVGQGISPEAFVEDADIREISATRRVDDSYANGYGNSKWAGEVLLREAHDWCGLPVSVFRCDMI
LADTTYSGQLNLPDMFTRLMLSLVATGIAPGSFYELDADGNRQRAHYDGLPVEFIAEAISTIGSQVTDGF
ETFHVMNPYDDGIGLDEYVDWLIEAGYPVHRVDDYATWLSRFETALRALPERQRQASLLPLLHNYQQPSP
PVCGAMAPTDRFRAAVQDAKIGPDKDIPHVTADVIVKYISNLQMLGLL
YP 889972(CARB)
Nucleotide sequence
>gi|118467340:5821317-5824838 Mycobacterium smegmatis str. MC2 155,
complete genome
(SEQ ID NO: 21)
ATGACCAGCGATGTTCACGACGCCACAGACGGCGTCACCGAAACCGCACTCGACGACGAGCAGTCGACCC
GCCGCATCGCCGAGCTGTACGCCACCGATCCCGAGTTCGCCGCCGCCGCACCGTTGCCCGCCGTGGTCGA
CGCGGCGCACAAACCCGGGCTGCGGCTGGCAGAGATCCTGCAGACCCTGTTCACCGGCTACGGTGACCGC
CCGGCGCTGGGATACCGCGCCCGTGAACTGGCCACCGACGAGGGCGGGCGCACCGTGACGCGTCTGCTGC
CGCGGTTCGACACCCTCACCTACGCCCAGGTGTGGTCGCGCGTGCAAGCGGTCGCCGCGGCCCTGCGCCA
CAACTTCGCGCAGCCGATCTACCCCGGCGACGCCGTCGCGACGATCGGTTTCGCGAGTCCCGATTACCTG
ACGCTGGATCTCGTATGCGCCTACCTGGGCCTCGTGAGTGTTCCGCTGCAGCACAACGCACCGGTCAGCC
GGCTCGCCCCGATCCTGGCCGAGGTCGAACCGCGGATCCTCACCGTGAGCGCCGAATACCTCGACCTCGC
AGTCGAATCCGTGCGGGACGTCAACTCGGTGTCGCAGCTCGTGGTGTTCGACCATCACCCCGAGGTCGAC
GACCACCGCGACGCACTGGCCCGCGCGCGTGAACAACTCGCCGGCAAGGGCATCGCCGTCACCACCCTGG
ACGCGATCGCCGACGAGGGCGCCGGGCTGCCGGCCGAACCGATCTACACCGCCGACCATGATCAGCGCCT
CGCGATGATCCTGTACACCTCGGGTTCCACCGGCGCACCCAAGGGTGCGATGTACACCGAGGCGATGGTG
GCGCGGCTGTGGACCATGTCGTTCATCACGGGTGACCCCACGCCGGTCATCAACGTCAACTTCATGCCGC
TCAACCACCTGGGCGGGCGCATCCCCATTTCCACCGCCGTGCAGAACGGTGGAACCAGTTACTTCGTACC
GGAATCCGACATGTCCACGCTGTTCGAGGATCTCGCGCTGGTGCGCCCGACCGAACTCGGCCTGGTTCCG
CGCGTCGCCGACATGCTCTACCAGCACCACCTCGCCACCGTCGACCGCCTGGTCACGCAGGGCGCCGACG
AACTGACCGCCGAGAAGCAGGCCGGTGCCGAACTGCGTGAGCAGGTGCTCGGCGGACGCGTGATCACCGG
ATTCGTCAGCACCGCACCGCTGGCCGCGGAGATGAGGGCGTTCCTCGACATCACCCTGGGCGCACACATC
GTCGACGGCTACGGGCTCACCGAGACCGGCGCCGTGACACGCGACGGTGTGATCGTGCGGCCACCGGTGA
TCGACTACAAGCTGATCGACGTTCCCGAACTCGGCTACTTCAGCACCGACAAGCCCTACCCGCGTGGCGA
ACTGCTGGTCAGGTCGCAAACGCTGACTCCCGGGTACTACAAGCGCCCCGAGGTCACCGCGAGCGTCTTC
GACCGGGACGGCTACTACCACACCGGCGACGTCATGGCCGAGACCGCACCCGACCACCTGGTGTACGTGG
ACCGTCGCAACAACGTCCTCAAACTCGCGCAGGGCGAGTTCGTGGCGGTCGCCAACCTGGAGGCGGTGTT
CTCCGGCGCGGCGCTGGTGCGCCAGATCTTCGTGTACGGCAACAGCGAGCGCAGTTTCCTTCTGGCCGTG
GTGGTCCCGACGCCGGAGGCGCTCGAGCAGTACGATCCGGCCGCGCTCAAGGCCGCGCTGGCCGACTCGC
TGCAGCGCACCGCACGCGACGCCGAACTGCAATCCTACGAGGTGCCGGCCGATTTCATCGTCGAGACCGA
GCCGTTCAGCGCCGCCAACGGGCTGCTGTCGGGTGTCGGAAAACTGCTGCGGCCCAACCTCAAAGACCGC
TACGGGCAGCGCCTGGAGCAGATGTACGCCGATATCGCGGCCACGCAGGCCAACCAGTTGCGCGAACTGC
GGCGCGCGGCCGCCACACAACCGGTGATCGACACCCTCACCCAGGCCGCTGCCACGATCCTCGGCACCGG
GAGCGAGGTGGCATCCGACGCCCACTTCACCGACCTGGGCGGGGATTCCCTGTCGGCGCTGACACTTTCG
AACCTGCTGAGCGATTTCTTCGGTTTCGAAGTTCCCGTCGGCACCATCGTGAACCCGGCCACCAACCTCG
CCCAACTCGCCCAGCACATCGAGGCGCAGCGCACCGCGGGTGACCGCAGGCCGAGTTTCACCACCGTGCA
CGGCGCGGACGCCACCGAGATCCGGGCGAGTGAGCTGACCCTGGACAAGTTCATCGACGCCGAAACGCTC
CGGGCCGCACCGGGTCTGCCCAAGGTCACCACCGAGCCACGGACGGTGTTGCTCTCGGGCGCCAACGGCT
GGCTGGGCCGGTTCCTCACGTTGCAGTGGCTGGAACGCCTGGCACCTGTCGGCGGCACCCTCATCACGAT
CGTGCGGGGCCGCGACGACGCCGCGGCCCGCGCACGGCTGACCCAGGCCTACGACACCGATCCCGAGTTG
TCCCGCCGCTTCGCCGAGCTGGCCGACCGCCACCTGCGGGTGGTCGCCGGTGACATCGGCGACCCGAATC
TGGGCCTCACACCCGAGATCTGGCACCGGCTCGCCGCCGAGGTCGACCTGGTGGTGCATCCGGCAGCGCT
GGTCAACCACGTGCTCCCCTACCGGCAGCTGTTCGGCCCCAACGTCGTGGGCACGGCCGAGGTGATCAAG
CTGGCCCTCACCGAACGGATCAAGCCCGTCACGTACCTGTCCACCGTGTCGGTGGCCATGGGGATCCCCG
ACTTCGAGGAGGACGGCGACATCCGGACCGTGAGCCCGGTGCGCCCGCTCGACGGCGGATACGCCAACGG
CTACGGCAACAGCAAGTGGGCCGGCGAGGTGCTGCTGCGGGAGGCCCACGATCTGTGCGGGCTGCCCGTG
GCGACGTTCCGCTCGGACATGATCCTGGCGCATCCGCGCTACCGCGGTCAGGTCAACGTGCCAGACATGT
TCACGCGACTCCTGTTGAGCCTCTTGATCACCGGCGTCGCGCCGCGGTCGTTCTACATCGGAGACGGTGA
GCGCCCGCGGGCGCACTACCCCGGCCTGACGGTCGATTTCGTGGCCGAGGCGGTCACGACGCTCGGCGCG
CAGCAGCGCGAGGGATACGTGTCCTACGACGTGATGAACCCGCACGACGACGGGATCTCCCTGGATGTGT
TCGTGGACTGGCTGATCCGGGCGGGCCATCCGATCGACCGGGTCGACGACTACGACGACTGGGTGCGTCG
GTTCGAGACCGCGTTGACCGCGCTTCCCGAGAAGCGCCGCGCACAGACCGTACTGCCGCTGCTGCACGCG
TTCCGCGCTCCGCAGGCACCGTTGCGCGGCGCACCCGAACCCACGGAGGTGTTCCACGCCGCGGTGCGCA
CCGCGAAGGTGGGCCCGGGAGACATCCCGCACCTCGACGAGGCGCTGATCGACAAGTACATACGCGATCT
GCGTGAGTTCGGTCTGATCTGA
Amino acid sequence
>gi|118469671|ref|YP_889972.1| putative long-chain fatty-acid--CoA ligase
[Mycobacterium smegmatis str. MC2 155]
(SEQ ID NO: 22)
MTSDVHDATDGVTETALDDEQSTRRIAELYATDPEFAAAAPLPAVVDAAHKPGLRLAEILQTLFTGYGDR
PALGYRARELATDEGGRTVTRLLPRFDTLTYAQVWSRVQAVAAALRHNFAQPIYPGDAVATIGFASPDYL
TLDLVCAYLGLVSVPLQHNAPVSRLAPILAEVEPRILTVSAEYLDLAVESVRDVNSVSQLVVFDHHPEVD
DHRDALARAREQLAGKGIAVTTLDAIADEGAGLPAEPIYTADHDQRLAMILYTSGSTGAPKGAMYTEAMV
ARLWTMSFITGDPTPVINVNFMPLNHLGGRIPISTAVQNGGTSYFVPESDMSTLFEDLALVRPTELGLVP
RVADMLYQHHLATVDRLVTQGADELTAEKQAGAELREQVLGGRVITGFVSTAPLAAEMRAFLDITLGAHI
VDGYGLTETGAVTRDGVIVRPPVIDYKLIDVPELGYFSTDKPYPRGELLVRSQTLTPGYYKRPEVTASVF
DRDGYYHTGDVMAETAPDHLVYVDRRNNVLKLAQGEFVAVANLEAVFSGAALVRQIFVYGNSERSFLLAV
VVPTPEALEQYDPAALKAALADSLQRTARDAELQSYEVPADFIVETEPFSAANGLLSGVGKLLRPNLKDR
YGQRLEQMYADIAATQANQLRELRRAAATQPVIDTLTQAAATILGTGSEVASDAHFTDLGGDSLSALTLS
NLLSDFFGFEVPVGTIVNPATNLAQLAQHIEAQRTAGDRRPSFTTVHGADATEIRASELTLDKFIDAETL
RAAPGLPKVTTEPRTVLLSGANGWLGRFLTLQWLERLAPVGGTLITIVRGRDDAAARARLTQAYDTDPEL
SRRFAELADRHLRVVAGDIGDPNLGLTPEIWHRLAAEVDLVVHPAALVNHVLPYRQLFGPNVVGTAEVIK
LALTERIKPVTYLSTVSVAMGIPDFEEDGDIRTVSPVRPLDGGYANGYGNSKWAGEVLLREAHDLCGLPV
ATFRSDMILAHPRYRGQVNVPDMFTRLLLSLLITGVAPRSFYIGDGERPRAHYPGLTVDFVAEAVTTLGA
QQREGYVSYDVMNPHDDGISLDVFVDWLIRAGHPIDRVDDYDDWVRRFETALTALPEKRRAQTVLPLLHA
FRAPQAPLRGAPEPTEVFHAAVRTAKVGPGDIPHLDEALIDKYIRDLREFGLI
YP 905678.1
Nucleotide sequence
>uniprot|A0PPD8|A0PPD8_MYCUA Fatty-acid-CoA ligase FadD9
(SEQ ID NO: 23)
ATGTCGCCAATCACGCGTGAAGAGCGGCTCGAGCGCCGCATCCAGGACCTCTACGCCAAC
GACCCGCAGTTCGCCGCCGCCAAACCCGTCACGGCGATCACCGCAGCAATCGAGCGGCCG
GGTCTACCGCTACCCCAGATCATCGAGACCGTCATGACCGGATACGCCGATCGGCCGGCT
CTCGCTCAGCGCTCGGTCGAATTCGTGACCGATGCCGGCACCGGCCACACCACGCTGCGA
CTGCTCCCCCACTTCGAAACCATCAGCTACGGCGAGCTTTGGGACCGCATCAGCGCACTG
GCCGACGTGCTCAGCACCGAACAGACGGTGAAACCGAGCGACCGGGTCTGCTTGTTGGGC
TTCAACAGCGTCGACTACGCCACGATCGACATGACTTTGGCGCGGCTGGGCGCGGTGGCT
GTACCACTGCAGACCAGCGCGGCGATAACCCAGCTGCAGCCGATCGTCGCCGAGACCCAG
CCCACCATGATCGCGGCCAGCGTCGACGCACTCGCTGACGCCACCGAATTGGCTCTGTCC
GGTCAGACCGCTACCCGAGTCCTGGTGTTCGACCACCACCGGCAGGTTGACGCACACCGC
GCAGCGGTCGAATCCGCCCGGGAGCGCCTGGCTGGCTCGGCGGTCGTCGAAACCCTGGCC
GAGGCCATCGCGCGCGGCGACGTGCCCCGCGGTGCGTCCGCCGGCTCGGCGCCCGGCACC
GATGTGTCCGACGACTCGCTCGCGCTACTGATCTACACCTCGGGCAGCACCGGTGCGCCC
AAGGGCGCGATGTACCCCCGACGCAACGTTGCGACCTTCTGGCGCAAGCGCACCTGGTTC
CAAGGCGGCTACGAGCcGTCGATCAcGGTGAACTTCATGCGAATGAGCcAGGTGATGGGC
CGCGAAATCCTGTACGGCACGCTGTGCAATGGCGGCACCGCCTACTTCGTGGTGAAAAGC
GATCTCTCCACCTTGTTCGAAGACCTGGCGCTGGTGCGGCCCACCGAGCTCACCTTCGTG
CCGCGCGTGTGGGACATGGTGTTCGACGAGTTTCAGAGTGAGGTCGACCGCCGCCTGGTC
GACGGCGCCGACCGGGTCGCGCTCGAAGCCCAGGTCAAGGCCGAGATACGCAACGACGTG
CTCGGTGGACGGTATACCAGCGCACTGACCGGCTCCGCCCCGATCTCCGACGAGATGAAG
GCGTGGGTCGAGGAGGTGGTCGACATGCATCTGGTCGAGGGCTACCGCTCCACCGAGGCC
GGGATGATCCTGATCGACGGAGCCATTCGGCGCCCGGCGGTACTCGACTACAAGCTGGTC
GATGTTCCCGACCTGGGTTACTTCCTGACCGACCGGCCACATCCGCGGGGCGAGTTGCTG
GTCAAGACCGATAGTTTGTTCCCGGGCTACTACCAGCGAGCCGAAGTCACCGCCGACGTG
TTCGATGCTGACGGCTTCTACCGGACCGGCGACATCATGGCCGAGGTCGGCCCCGAACAG
TTCGTGTACCTCGACCGCCGCAACAACGTGTTGAAGCTGTCGCAGGGCGAGTTCGTCACC
GTCTCCAAACTCGAAGCGGTGTTTGGCGACAGCCCACTGGTACGGCAGATCTACATCTAC
GGCAACAGCGCCCGTGCCTACCTGTTGGCGGTGATCGTCCCCACCCAGGAGGCGCTGGAC
GCCGTGCCTGTCGAGGAGCTCAAGGCGCGGCTGGGCGACTCGCTGCAAGAGGTCGCAAAG
GCCGCCGGCCTGCAGTCCTACGAGATCCCGCGCGACTTCATCATCGAAACAACACCATGG
ACGCTGCAGAACGGCCTGCTCACCGGCATCCGCAAGTTGGCCAGGCCGCAGCTGAAAAAG
CATTACGGCGAGCTTCTCGAGCAGATCTACACGGACCTGGCACACGGCCAGGCCGACGAA
CTGCGCTCGCTGCGCCAAAGCGGTGCCGATGCGCCGGTGCTGGTGACGGTGTGCCGCGCG
GCGGCCGCGCTGTTGGGCGGCAGCGCCTCTGACGTCCAGCCCGATGCGCACTTCACCGAT
TTGGGCGGCGACTCGCTGTCGGCGCTGTCGTTCACCAACCTGCTGCACGAGATCTTCGAC
ATCGATGTGCCGGTGGGCGTCATCGTCAGCCCCGCCAACGACTTGCAGGCCCTGGCCGAC
TACGTCGAGGCGGCTCGCAAACCCGGCTCGTCACGACCGACCTTCGCCTCGGTCCACGGC
GCCTCGAATGAGCAGGTCACCGAGGTGCATGCCGGTGACCTGTCCCTGGACAAATTCATC
GATGCCGCAACCCTGGCCGAAGCTCCCCGGCTGCCCGCCGCAAACACCCAAGTGCGCACC
GTGCTGCTGACCGGCGCCACCGGCTTCCTCGGGCGCTACCTGGCCCTGGAATGGCTGGAG
CGGATGGACCTGGTCGACGGCAAACTGATCTGCCTGGTCCGGGCCAAGTCCGACACCGAA
GCACGGGCGCGGCTGGAAAAGACGTTCGACAGCGGCGCCCCCGAACTGCTGGCCCACTAC
CGCGCACTGGCCGGCGACCACCTCGAGGTGCTCGCCGGTGACAAGGGCGAAGCCGACCTC
GGACTGGACCGGCAGACCTGGCAACGCCTGGCCGACACGGTCGACCTGATCGTGGACCCC
GCGGCCCTGGTCAACCACGTACTGCCATACAGCCAGCTGTTCGGGCCCAACGCGCTGGGC
ACCGCCGAGCTGCTGCGGCTCGCGCTCACCTCCAAGATCAAGCCCTACAGCTACACCTCG
ACAATCGGTGTCGCCGACCAGATCCCGCCGTCGGCGTTCACCGAGGACGCCGACATCCGG
GTCATCAGCGCCACCCGCGCGGTCGACGACAGCTACGCCAATGGCTATTCGAACAGCAAG
TGGGCCGGCGAGGTGCTGTTGCGCGAGGCGCATGTCCTGTGTGGCCTGCCGGTTGCGGTG
TTCCGCTGCGACATGATCCTGGCCGACACCACATGGGCGGGACAGCTCAACGTGCCGGAC
ATGTTCACCCGTATGATCCTGAGCCTGGCGGCCACCGGTATCGCGCCGGGTTCGTTCTAT
GAGCTTGCGGCCGACGGCGCCCGGCAACGCGCCCACTATGACGGTCTGCCCGTCGAGTTC
ATCGCCGAGGCGATTTCGACTTTGGGTGCGCAGAGCCAGGATGGGTTCCACACGTATCAC
GTGATGAACCCTTACGACGACGGCATCGGACTCGACGAGTTCGTCGACTGGCTCAACGAG
TCCGGTTGCCCCATCCAGCGCATCGCTGACTATGGCGACTGGCTGCAGCGCTTCGAAACC
GCACTGCGCGCACTGCCCGATCGGCAGCGGCACAGCTCACTGCTGCCGCTGTTGCACAAC
TATCGGCAGCCGGAGCGGCCCGTCCGCGGGTCGATCGCCCCTACCGATCGCTTCCGGGCA
GCGGTGCAAGAGGCCAAGATCGGCCCCGACAAAGACATTCCGCACGTCGGCGCGCCGATC
ATCGTGAAGTACGTCAGCGACCTGCGCCTACTCGGCCTGCTCTGA
Amino acid sequence
>uniprot|A0PPD8|A0PPD8_MYCUA Fatty-acid-CoA ligase FadD9
(SEQ ID NO: 24)
MSPITREERLERRIQDLYANDPQFAAAKPVTAITAAIERPGLPLPQIIET
VMTGYADRPALAQRSVEFVTDAGTGHTTLRLLPHFETISYGELWDRISAL
ADVLSTEQTVKPSDRVCLLGFNSVDYATIDMTLARLGAVAVPLQTSAAIT
QLQPIVAETQPTMIAASVDALADATELALSGQTATRVLVFDHHRQVDAHR
AAVESARERLAGSAVVETLAEAIARGDVPRGASAGSAPGTDVSDDSLALL
IYTSGSTGAPKGAMYPRRNVATFWRKRTWFEGGYEPSITLNFMPMSHVMG
RQILYGTLCNGGTAYFVVKSDLSTLFEDLALVRPTELTFVPRVWDMVFDE
FQSEVDRRLVDGADRVALEAQVKAEIRNDVLGGRYTSALTGSAPISDEMK
AWVEELLDMHLVEGYGSTEAGMILIDGAIRRPAVLDYKLVDVPDLGYFLT
DRPHPRGELLVKTDSLFPGYYQRAEVTADVFDADGFYRTGDIMAEVGPEQ
FVYLDRRNNVLKLSQGEFVTVSKLEAVFGDSPLVRQIYIYGNSARAYLLA
VIVPTQEALDAVPVEELKARLGDSLQEVAKAAGLQSYEIPRDFIIETTPW
TLQNGLLTGIRKLARPQLKKHYGELLEQIYTDLAHGQADELRSLRQSGAD
APVLVTVCRAAAALLGGSASDVQPDAHFTDLGGDSLSALSFTNLLHEIFD
IDVPVGVIVSPANDLQALADYVEAARKPGSSRPTFASVHGASNEQVTEVH
AGDLSLDKFIDAATLAEAPRLPAANTQVRTVLLTGATGFLGRYLALEWLE
RMDLVDGKLICLVRAKSDTEARARLEKTFDSGAPELLAHYRALAGDHLEV
LAGDKGEADLGLDRQTWQRLADTVDLIVDPAALVNHVLPYSQLFGPNALG
TAELLRLALTSKIKPYSYTSTIGVADQIPPSAFTEDADIRVISATRAVDD
SYANGYSNSKWAGEVLLREAHVLCGLPVAVFRCDMILADTTWAGQLNVPD
MFTRMILSLAATGIAPGSFYELAADGARQRAHYDGLPVEFIAEAISTLGA
QSQDGFHTYHVMNPYDDGIGLDEFVDWLNESGCPIQRIADYGDWLQRFET
ALRALPDRQRHSSLLPLLHNYRQPERPVRGSIAPTDRFRAAVQEAKIGPD
KDIPHVGAPIIVKYVSDLRLLGLL

### Amino Acid Sequence Motifs 1

### Amino Acid Sequence Motifs for CAR Homologs

### Motif 1

### -G-Y-X-X-S/A/T-K-W/L (SEQ ID NO:7); and

### -G-X-X-G-X-L-G (SEQ ID NO:8); and

### -L/V/1-G-G-D-S-X-X-A (SEQ ID NO:9); and

-[LIVMFY]-{E}-{VES}-[STG]-[STAG]-G-[ST]-[STEIA]-[SG]-X-[PASLIVM]-[KR] (SEQ ID NO:10), where {X} stands for any amino acid except X and [X₁X₂] stands for X₁ or X₂

### Motif 2

(SEQ ID NO: 11)
RTVLLX₁GAX₂GX₃LGRX₄LX₅LX₆WL

where
X₁ is S or T;
X₂ is T or N;
X₃ is F or W;
X₄ is F or Y;
X₅ is A or T; and
X₆ is E or Q

### Motif 3

(SEQ ID NO: 12)
LXXGXXGXLGXXLXLXWLXR

### Motif 4

WAXEVLLR (SEQ ID NO:13), where X can be any amino acid; or LXXGXXGXLGXXLXX₁XX₂LX₃R (SEQ ID NO:14), where

X₁ is Leu or Ile;

X₂ is Trp or Leu; and

X₃ varies between 13 amino acids or 14 amino acids

### Motif 5

### -G-Y-X-X-S/A/T-K-W/L (SEQ ID NO:7); and

### -L/V/1-G-G-D-S-X-X-A (SEQ ID NO:9); and

-[LIVMFY]-{E}-{VES}-[STG]-[STAG]-G-[ST]-[STEIA]-[SG]-X-[PASLIVM]-[KR] (SEQ ID NO:10), where {X} stands for any amino acid except X and [X₁X₂] stands for X₁ or X₂; and RTVLLX₁GAX₂GX₃LGRX₄LX₅LX₆WL (SEQ ID NO:11), where

X₁ is S or T:

X₂ is T or N;

X₃ is F or W;

X₄ is F or Y;

X₅ is A or T; and

X₆ is E or Q

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A detergent composition comprising a microbially produced fatty alcohol or fatty alcohol derivative thereof.

2. The detergent composition of claim 1, wherein the microbially produced fatty alcohol or fatty alcohol derivative thereof is prepared by a method comprising expressing a gene encoding a recombinant acyl-CoA synthase in the host cell.

3. The detergent composition of claim 1, wherein the detergent composition comprises from about 0.05% to about 70 wt % of microbially produced fatty alcohol or fatty alcohol derivative thereof.

4. The detergent composition of claim 3, wherein the fatty alcohol derivative thereof comprises a fatty ether sulfate, fatty alcohol sulfate, a fatty phosphate ester, an alkylbenzyldimethylammonium chloride, a fatty amine oxide, an alkyl polyglucoside, an alkyl glyceryl ether sulfonate, a fatty alcohol alkoxylate, or a combination thereof.

5. The detergent composition of claim 4, wherein the fatty alcohol alkoxylate is an ethoxylated fatty alcohol.

6. The detergent composition of claim 1, wherein the fatty alcohol or fatty alcohol derivative thereof comprises a saturated, monounsaturated, or polyunsaturated fatty alcohol.

7. The detergent composition of claim 6, wherein the fatty alcohol or fatty alcohol derivative thereof is monounsaturated at the omega-7 position.

8. The detergent composition of claim 1, wherein the detergent composition comprises from about 0.1% to about 40 wt % of microbially produced fatty alcohol or fatty alcohol derivative thereof.

9. The detergent composition of claim 1, wherein the detergent composition comprises from about 0.25% to about 10 wt % of microbially produced fatty alcohol or fatty alcohol derivative thereof.

10. The detergent composition of claim 1, wherein the fatty alcohol comprises a C₆-C₂₆ fatty alcohol.

11. The detergent composition of claim 1, wherein the microbially produced fatty alcohol or fatty alcohol derivative thereof is prepared by a method comprising expressing a gene encoding a recombinant alcohol dehydrogenase in the host cell.

12. The detergent composition of claim 11, wherein the alcohol dehydrogenase is AlrA, YjgB, YahK, or AlrAadp1.

13. The detergent composition of claim 1, wherein the host cell is genetically engineered to express a decreased level of an acyl-CoA synthase relative to a wild-type host cell.

14. The detergent composition of claim 13, wherein the genetically engineered host cell comprises a knockout of an acyl-CoA synthase gene.

15. The detergent composition of claim 14, wherein the acyl-CoA synthase gene is FadD.

16. The detergent composition of claim 1, wherein the microbially produced fatty alcohol is prepared by a method further comprising expressing a gene encoding a recombinant acyl-CoA reductase in the host cell.

17. The detergent composition of claim 16, therein the acyl-CoA reductase is Acr1, mFAR1, mFAR2, JjFAR, BmFAR, AcrM, or hFAR.

18. The detergent composition of claim 16, wherein the method further comprises converting the isolated fatty alcohol into a fatty alcohol derivative thereof.

19. The detergent composition of claim 16, wherein the fatty alcohol or fatty alcohol derivative thereof is monounsaturated at the omega-7 position.

20. The detergent composition of claim 16, wherein the microbially produced fatty alcohol or fatty alcohol derivative thereof is prepared by a method comprising expressing a gene encoding a recombinant alcohol dehydrogenase in the host cell.

21. The detergent composition of claim 20, wherein the alcohol dehydrogenase is AlrA, YjgB, YahK, or AlrAadp1.
